(19) United States
(12) Patent Application Publication
(10) Pub. No.: US 2009/0128767 A1
(43) Pub. Date: May 21, 2009

(54) Curable resin composition for column spacer,column spacer, and liquid crystal display panel

(76) Inventors: Minoru Suezaki, Osaka (JP); Yoshio Nishimura, Osaka (JP); Toru Takahashi, Osaka (JP); Sayaka Kobayashi, Osaka (JP); Tatsuya Matsukubo, Osaka (JP)

Correspondence Address: WENDEROTH, LIND & PONACK, L.L.P., 1030 15th Street, N.W.,, Suite 400 East, Washington, DC, 20005-1503, US

(21) Appl. No.: 11/921,155

(22) PCT Filed: May 30, 2006
(86) PCT No.: PCT/JP2006/310786

§ 371 Date: Feb. 8, 2008

(30) Foreign Application Priority Data
May 30, 2005 (JP) 2005-157954
Jun. 23, 2005 (JP) 2005-183217
Aug. 2, 2005 (JP) 2005-224004
Aug. 5, 2005 (JP) 2005-228629

(51) Int. Cl. G02F 1/1339; C08F 2/46; C08F 20/10; C07C 69/73

(52) U.S. Cl. 349/155; 522/178; 522/182; 522/183; 522/168; 522/180; 522/173; 560/183

(57) ABSTRACT

It is an object of the present invention to provide a curable resin composition for a column spacer which has excellent developability and solubility and is capable of forming a clearly patterned column spacer without leaving a development residue at the time of pattern formation of the column spacer to be used in producing a liquid crystal display panel.

The present invention is directed to a curable resin composition for a column spacer, which contains a compound having two or more polymerizable unsaturated bonds in a molecule, an alkali-soluble polymer compound, and a photo-reaction initiator, the compound having two or more polymerizable unsaturated bonds in a molecule being an oxide-modified compound having two or more polymerizable unsaturated bonds in a molecule.

### TECHNICAL FIELD

The present invention relates to a curable resin composition for a column spacer which has excellent developability and solubility and is capable of forming a clearly patterned column spacer without leaving a development residue at the time of pattern formation of the column spacer to be used in producing a liquid crystal display panel; a curable resin composition for a column spacer which is capable of forming a clearly patterned column spacer without leaving a development residue at the time of pattern formation of the column spacer to be used in producing a liquid crystal display panel and capable of obtaining a liquid crystal display panel capable of effectively suppressing occurrence of color irregularity due to gravity defect without generating cold bubble; a column spacer obtained by using the curable resin composition for a column spacer; and a liquid crystal display panel.

### BACKGROUND ART

Generally, a liquid crystal display panel comprises a spacer for keeping the gap of two glass substrates constant and a transparent electrode, a polarizer, an alignment layer for aligning a liquid crystal substance, and the like besides the spacer. Presently as the spacer, a fine particle spacer mainly having particle diameter of several μm has been used. However in a conventional method of producing a liquid crystal display panel, since the fine particle spacer is scattered randomly on a glass substrate, the fine particle spacer is sometimes arranged in pixel parts. If the fine particle spacer exists in the pixel parts, due to the disorder of the liquid crystal alignment in the surrounding of the spacer, the light leaks to cause problems that the quality of images decreases such as low contrast of an image. To deal with the problems, fine particle spacer arrangement methods for avoiding the fine particle spacers to exist in the pixel parts have been investigated, however the methods are all complicated in the operation and insufficient for practical applications.

Further, to increase the productivity of the liquid crystal display panel, One prop Fill Technology (ODF method) has been recently proposed. The method is a method for producing a liquid crystal display panel by dropping a predetermined amount of a liquid crystal on a liquid crystal enclosing face of a glass substrate, setting another substrate for a liquid crystal panel on the opposite in vacuum in a state that a predetermined cell gap can be maintained, and sticking both substrates to each other. Accordingly, this method enables the liquid crystal display panel to have an enlarged surface area and makes it easy to enclose the liquid crystal even if the cell gap is narrowed as compared with conventional methods and for that, the ODF method is supposed to become main stream for the method of producing a liquid crystal display panel.

However, if the fine particle spacer is used in the ODF method, at the time of the drop of the liquid crystal or sticking mutually opposed substrates, the scattered fine particle spacer flow together with the flotation of the liquid crystal and thus there occurs a problem that the distribution of the fine particle spacer on the substrate becomes uneven. If the distribution of the fine particle spacer becomes uneven, cell gaps of liquid crystal cells become uneven and it causes a problem of color irregularity of the liquid crystal display.

To deal with that problem, a column spacer formed a convex pattern for evenly keeping cell gaps by photolithographic technique on a liquid crystal substrate were proposed in place of the conventional fine particle spacer and have been used practically (e.g., reference to Patent Document 1 and Patent Document 2).

If such a column spacer is used, the problems that the spacer is disposed in pixel parts and that spacer irregularity occurs in ODF method are solved.

Further, with respect to a large scale liquid crystal display panel produced by using a column spacer of a conventional resin composition for a column spacer by ODF method, since the liquid crystal in a liquid crystal cell fluidizes downward during the use of the liquid crystal display panel, a defect, so-called “gravity defect”, that is, color irregularity in an upper half face and a lower half face of display panel, is sometimes caused and it becomes a big issue. It is supposed that the “gravity defect” phenomenon is caused since the liquid crystal in a liquid crystal cell is expanded due to the heat generated by backlight, widens the cell gap and allows the lift of the substrate from the column spacers at that time and accordingly a portion of the liquid crystal corresponding to the volume which is not held by the spacers fluidizes downward by the gravity.

To solve such “gravity defect”, it is supposed to be possible to solve the problem by enabling the column spacer, which is once compacted, to follow the alteration of the cell gap by elastically recovering the compaction deformation of the column spacer and thus forming no gap between the substrate and the column spacer at the time when the cell gap is widened because of the expansion of the liquid crystal in the liquid crystal cell due to the heat generated by the backlight.

However, in the case of conventional methods, to give high deformation recovery force to the column spacer, it is required to crosslink the resin forming the column spacer to so high extent as to make plastic deformation hardly occur in the compressing. However, a resin having such a highly crosslinked structure generally tends to have high compressive elastic modulus and be hard. In the case the column spacer is formed using such a hard resin, high pressure is needed in the process of compressive deformation of the column spacer and in the obtained liquid crystal display panel, high force of widening the liquid crystal cell by the compacted column spacer is enclosed. In the case such force of the column spacer to widen the liquid crystal cell is high, there occurs a problem that a phenomenon, so-called “cold bubble” is caused, that foams are generated due to abrupt decrease of the inner pressure in the liquid crystal cell in the case volumetric contraction of the liquid crystal occurs in the liquid crystal cell at the time of a low temperature.

Patent Document 1: Japanese Kokai Publication 2001-91954

Patent Document 2: Japanese Kokai Publication 2002-251007

### DISCLOSURE OF THE INVENTION

### Problems to be Solved by the Invention

In view of the above-mentioned state of the art, the present invention aims to provide a column spacer which has excellent developability and solubility and is capable of forming a clearly patterned column spacer without leaving a development residue at the time of pattern formation of the column spacer to be used in producing a liquid crystal display panel; a curable resin composition for a column spacer which is capable of forming a clearly patterned column spacer without leaving a development residue at the time of pattern formation of the column spacer to be used in producing a liquid crystal display panel and capable of obtaining a liquid crystal display panel capable of effectively suppressing occurrence of color irregularity due to gravity defect without generating cold bubble; a column spacer obtained by using the curable resin composition for a column spacer; and a liquid crystal display panel.

### Means for Solving the Problems

The first present invention is a curable resin composition for a column spacer, which contains a compound having two or more polymerizable unsaturated bonds in a molecule, an alkali-soluble polymer compound, and a photo-reaction initiator, the compound having two or more polymerizable unsaturated bonds in a molecule being an oxide-modified compound having two or more polymerizable unsaturated bonds in a molecule.

The second present invention is a curable resin composition for a column spacer, which contains a compound having two or more polymerizable unsaturated bonds in a molecule, an alkali-soluble polymer compound, and a photo-reaction initiator, the compound having two or more polymerizable unsaturated bonds in a molecule being an oxide-modified compound having one or more hydroxyl group and two or more polymerizable unsaturated bonds in a molecule.

The third present invention is a curable resin composition for a column spacer, which contains a compound having two or more polymerizable unsaturated bonds in a molecule, an alkali-soluble polymer compound, and a photo-reaction initiator, the compound having two or more polymerizable unsaturated bonds in a molecule being a lactone-modified and oxide-modified compound having two or more polymerizable unsaturated bonds in a molecule.

The fourth present invention is a curable resin composition for a column spacer, which contains a compound having two or more polymerizable unsaturated bonds in a molecule, an alkali-soluble polymer compound, and a photo-reaction initiator, the compound having two or more polymerizable unsaturated bonds in a molecule being a lactone-modified compound having one or more hydroxyl group and two or more polymerizable unsaturated bonds in a molecule.

The fifth present invention is a curable resin composition for a column spacer, which contains a compound having two or more polymerizable unsaturated bonds in a molecule, an alkali-soluble polymer compound, and a photo-reaction initiator, the compound having two or more polymerizable unsaturated bonds in a molecule being a lactone-modified and oxide-modified compound having one or more hydroxyl group and two or more polymerizable unsaturated bonds in a molecule.

The sixth present invention is a curable resin composition for a column spacer, which contains a compound having two or more polymerizable unsaturated bonds in a molecule, an alkali-soluble polymer compound, and a photo-reaction initiator, the compound having two or more polymerizable unsaturated bonds in a molecule being a compound having one or more carboxyl group and two or more polymerizable unsaturated bonds in a molecule.

Hereinafter, the present invention will be described more in detail.

The present inventors found, as a result of diligent examination, that it is possible to form a column spacer in a clear pattern with excellent resolution at the time of pattern formation of the column spacer by using a compound having two or more polymerizable unsaturated bonds in a molecule with a specified structure and an alkali-soluble polymer compound in combination as a curable resin for a column spacer and also it is possible to obtain a column spacer with excellent flexibility and high compression recovery property and these findings have now led to completion of the invention. According to the column spacer obtained by using the curable resin composition for a column spacer of the present invention, it is possible to simultaneously suppress “gravity defect” due to liquid crystal expansion at the time of heating and “cold bubble” due to contraction of the liquid crystal at the time of low temperature and at the time of pattern formation to form a column spacer by the photolithographic technique, a sharp resolution can be obtained without leaving a development residue.

The curable resin composition for a column spacer of the first present invention contains a compound having two or more polymerizable unsaturated bonds in a molecule, an alkali-soluble polymer compound, and a photo-reaction initiator.

In the curable resin composition for a column spacer of the first present invention, the compound having two or more polymerizable unsaturated bonds in a molecule is an oxide-modified compound having two or more polymerizable unsaturated bonds in a molecule.

The oxide-modified compound having two or more polymerizable unsaturated bonds in a molecule (hereinafter, referred to also as a polymerizable compound according to the first present invention), which is not particularly limited, is preferably an oxide-modified polyfunctional (meth)acrylate compound (hereinafter, referred to also as a polyfunctional (meth)acrylate according to the first present invention). The curable resin composition for a column spacer of the first present invention containing the polymerizable compound according to the first present invention is excellent in the recovery property of the column spacer obtained by using the curable resin composition for a column spacer from the compressive deformation and is capable of simultaneously suppressing “gravity defect” due to liquid crystal expansion at the time of heating and “cold bubble” due to contraction of the liquid crystal at the time of low temperature in a liquid crystal display panel produced by using the column spacer. Further, at the time of pattern formation to form a column spacer by the photolithographic technique, a sharp resolution can be obtained without leaving a development residue.

In this description, in the case where the polymerizable compound according to the first present invention is a polyfunctional (meth)acrylate according to the first present invention, “oxide-modified” means a introduction of a ring-opened structure and/or a ring-opened polymer structure between an alcohol-derived portion of the (meth)acrylate compound and the (meth)acryloyl group. In this description, (meth)acrylate means acrylate or methacrylate.

Non-limiting examples of the oxide include ethylene oxide, propylene oxide, 1,2-butylene oxide, 2,3-butylene oxide, 1,3-butylene oxide, oxetane, tetrahydrofuran, 3-methyltetrahydrofuran, styrene oxide, α-olefin oxide, and epichlorohydrin. Among them, ethylene oxide and propylene oxide are used preferably. These oxides may be used alone or two or more of them may be used in combination.

Non-limiting examples of the polyfunctional (meth)acrylate according to the first present invention include compounds obtained by oxide-modifying difunctional (meth)acrylate compounds such as neopentyl glycol di(meth)acrylate, 3-methyl-1,5-pentanediol di(meth)acrylate, 2-butyl-2-ethyl-1,3-propanediol di(meth)acrylate, 1,4-butanediol diacrylate, 1,6-hexanediol diacrylate, hydroxypivalic acid neopentyl glycol ester diacrylate, trimethylolpropane di(meth)acrylate, trimethylolethane di(meth)acrylate, pentaerythritol di(meth)acrylate, ditrimethylolpropane di(meth)acrylate, and dipentaerythritol di(meth)acrylate; and compounds obtained by oxide-modifying tri- or higher-functional (meth)acrylate compounds such as trimethylolpropane tri(meth)acrylate, trimethylolethane tri(meth)acrylate, pentaerythritol tri(meth)acrylate, pentaerythritol tetra(meth)acrylate, ditrimethylolpropane tri(meth)acrylate, ditrimethylolpropane tetra(meth)acrylate, dipentaerythritol tri(meth)acrylate, dipentaerythritol tetra(meth)acrylate, dipentaerythritol penta(meth)acrylate, and dipentaerythritol hexa(meth)acrylate. Among them, compounds obtained by ethylene oxide-modifying and/or propylene oxide-modifying tri- or higher-functional (meth)acrylate compounds are preferable in particular since the polymerization reaction of these compounds proceeds quickly and the exposure sensitivity is easy to be improved. These polyfunctional (meth)acrylate compounds according to the first present invention may be used alone or two or more of them may be used in combination.

The modification degree of the oxide-modification of the polyfunctional (meth)acrylate according to the first present invention is preferably 0.5 n mole in the lower limit and 10 n mole in the upper limit to 1 mole of the polyfunctional (meth)acrylate compound, wherein n means the number of the functional groups of the polyfunctional (meth)acrylate compound as the base. If it is lower than 0.5 n mole, the resolution and solubility may sometimes become insufficient at the time of development and if it exceeds 10 n mole, the affinity with an alkaline developer solution increases and resolution tends to easily decrease due to swelling. It is more preferably 1 n mole in the lower limit and 5 n mole in the upper limit.

A practical method of synthesizing the polyfunctional (meth)acrylate according to the first present invention by oxide-modifying the polyfunctional (meth)acrylate compound is not particularly limited and examples are a method involving synthesizing an oxide-modified alcohol by reaction of a polyhydric alcohol and an oxide and successively esterifying the oxide-modified alcohol and (meth)acrylic acid.

With respect to the curable resin composition for a column spacer of the first present invention, the content of the polymerizable compound according to the first present invention is not particularly limited, however it is preferably 20% by weight in the lower limit and 90% by weight in the upper limit to the solid matter of the curable resin composition for a column spacer of the first present invention. If it is lower than 20% by weight, the curable resin composition for a column spacer of the first present invention is not sufficiently photo-cured and accordingly it may be sometimes impossible to form a pattern of the column spacer by photolithography and if it exceeds 90% by weight, the solubility in an alkaline developer solution becomes insufficient at the time of column spacer production using the curable resin composition for a column spacer of the first present invention and accordingly the developability of the pattern of the column spacer to be produced may sometimes become insufficient. It is more preferably 40% by weight in the lower limit and 80% by weight in the upper limit.

The curable resin composition for a column spacer of the first present invention may contain a not oxide-modified compound having a polymerizable unsaturated bond (hereinafter, simply, referred to also as a polymerizable unsaturated bond-containing compound), in order to adjust the reactivity, the developability and the like, in addition to the polymerizable compound according to the first present invention to an extent that the flexibility of the column spacer to be produced is not deteriorated.

Non-limiting examples of the polymerizable unsaturated bond-containing compound may be, as a difunctional compound, polyethylene glycol (meth)acrylates such as neopentyl glycol di(meth)acrylate, 3-methyl-1,5-pentanediol di(meth)acrylate, 2-butyl-2-ethyl-1,3-propanediol di(meth)acrylate, 1,4-butanediol diacrylate, 1,6-hexanediol diacrylate, hydroxypivalic acid neopentyl glycol ester diacrylate, diethylene glycol (meth)acrylate, triethylene glycol (meth)acrylate, tetraethylene glycol (meth)acrylate, hexaethylene glycol (meth)acrylate, and nonaethylene glycol (meth)acrylate; and polyethylene glycol di(meth)acrylates such as diethylene glycol di(meth)acrylate, triethylene glycol di(meth)acrylate, tetraethylene glycol di(meth)acrylate, hexaethylene glycol di(meth)acrylate, and nonaethylene glycol di(meth)acrylate.

Examples as tri- or higher-functional compounds may be polyfunctional (meth)acrylate compounds such as trimethylolethane tri(meth)acrylate, trimethylolpropane tri(meth)acrylate, ditrimethylolpropane tetra(meth)acrylate, pentaerythritol tri(meth)acrylate, pentaerythritol tetra(meth)acrylate, dipentaerythritol tetra(meth)acrylate, dipentaerythritol penta(meth)acrylate, and dipentaerythritol hexa(meth)acrylate.

In the case the curable resin composition for a column spacer of the first present invention contains the polymerizable unsaturated bond-containing compound, the addition amount of the compound is not particularly limited, however, the total amount of the compound and the polymerizable compound according to the first present invention is preferably lower than 40% by weight. If it exceeds 40% by weight, the flexibility of the column spacer to be produced is deteriorated and the effect of suppressing the gravity defect and cold bubble tends to be lowered. It is preferably 30% by weight in the upper limit.

The curable resin composition for a column spacer of the first present invention contains an alkali-soluble polymer compound.

Although the alkali-soluble polymer compound is not particularly limited, carboxyl-containing alkali-soluble polymer compound having a carboxyl group is preferably. Examples of the carboxyl-containing alkali-soluble polymer compound may be a copolymer obtained by copolymerizing a carboxyl-containing monofunctional unsaturated compound, a monofunctional compound having a reactive functional group such as epoxy group, and compounds having an unsaturated double bond (hereinafter, simply, referred to also as a copolymer). Further, commercialized products such as Cyclomer P, manufactured by Daicel Chem. Ind., Ltd. may be also used.

Non-limited examples of the carboxyl-containing monofunctional unsaturated compound are acrylic acid and methacrylic acid.

Non-limited examples of the monofunctional compound having an epoxy group are glycidyl acrylate, glycidyl methacrylate, glycidyl α-ethylacrylate, glycidyl α-n-propylacrylate, glycidyl α-n-butylacrylate, 3,4-epoxybutyl acrylate, 3,4-epoxybutyl methacrylate, 6,7-epoxyheptyl acrylate, 6,7-epoxyheptyl methacrylate, 6,7-epoxyheptyl α-ethylacrylate, o-vinylbenzyl glycidyl ether, m-vinylbenzyl glycidyl ether, p-vinylbenzyl glycidyl ether, and compounds defined by the following formula (1). Among them, glycidyl methacrylate, 6,7-epoxyheptyl methacrylate, o-vinylbenzyl glycidyl ether, m-vinylbenzyl glycidyl ether, and p-vinylbenzyl glycidyl ether are used preferably since they have high copolymerization reactivity and increase the strength of column spacer to be obtained. They may be used alone or two or more of them may be used in combination.

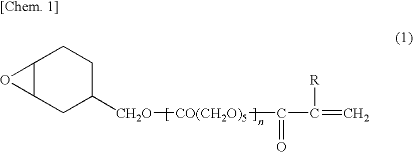

In the formula (1), R denotes hydrogen or an alkyl group having 1 to 5 carbon atoms; and n denotes an integer of 0 to 10.

Non-limiting examples of the copolymer are (meth)acrylic acid alkyl esters such as methyl (meth)acrylate, ethyl (meth)acrylate, propyl (meth)acrylate, n-butyl (meth)acrylate, sec-butyl (meth)acrylate, and tert-butyl (meth)acrylate; (meth)acrylic acid alkyl esters such as methyl (meth)acrylate and isopropyl (meth)acrylate; (meth)acrylic acid cyclic alkyl esters such as cyclohexyl (meth)acrylate, 2-methylcyclohexyl (meth)acrylate, dicyclopentanyl (meth)acrylate, dicyclopentanyloxyethyl (meth)acrylate, and isoboronyl (meth)acrylate; (meth)acrylic acid cyclic alkyl esters such as cyclohexyl (meth)acrylate, 2-methylcyclohexyl (meth)acrylate, dicyclopentanyl (meth)acrylate, dicyclopentaoxyethyl (meth)acrylate, and isoboronyl (meth)acrylate; (meth)acrylic acid aryl esters such as phenyl (meth)acrylate and benzyl (meth)acrylate; (meth)acrylic acid aryl esters such as phenyl (meth)acrylate and benzyl (meth)acrylate; dicarboxylic acid diesters such as diethyl maleate, diethyl fumarate, and diethyl itaconate; hydroxyalkyl esters such as 2-hydroxyethyl (meth)acrylate and 2-hydroxypropyl (meth)acrylate; styrene, α-methylstyrene, m-methylstyrene, p-methylstyrene, vinyltoluene, p-methoxystyrene, acrylonitrile, methacrylonitrile, vinyl chloride, vinylidene chloride, acrylamide, methacrylamide, vinyl acetate, 1,3-butadiene, isoprene, and 2,3-dimethyl-1,3-butadiene. Among them, styrene, tert-butyl (meth)acrylate, dicyclopentanyl (meth)acrylate, p-methoxystyrene, 2-methylcyclohexyl (meth)acrylate, and 1,3-butadiene are preferable in terms of the copolymerization reactivity and solubility in an alkaline aqueous solution. They may be used alone or two or more of them may be used in combination.

With respect to the copolymers, the ratio of the component derived from the carboxyl-containing monofunctional unsaturated compound is preferably 10% by weight in the lower limit and 40% by weight in the upper limit. If it is less than 10% by weight, it is difficult to provide alkali-solubility and if it exceeds 40% by weight, the swelling becomes significant at the time of development in the case of producing the column spacer using the curable resin composition for a column spacer of the first present invention and the formation of the column spacer pattern may sometimes become difficult. It is more preferably 15% by weight in the lower limit and 30% by weight in the upper limit.

The weight average molecular weight of the copolymer is not particularly limited, however, it is preferably 3000 in the lower limit and 100000 in the upper limit. If it is less than 3000, the developability may be lowered in the case of producing the column spacer using the curable resin composition for a column spacer of the first present invention, and if it exceeds 100000, the resolution may be lowered in the case of producing the column spacer using the curable resin composition for a column spacer of the first present invention. It is more preferably 5000 in the lower limit and 50000 in the upper limit.

A method of copolymerizing the carboxyl-containing monofunctional unsaturated compound and the monofunctional compound having a reactive functional group such as an unsaturated double bond or an epoxy group is not particularly limited and examples of the method may include conventional polymerization methods of mass polymerization, solution polymerization, suspension polymerization, dispersion polymerization, and emulsion polymerization using the radical polymerization initiator and if necessary molecular weight adjustment agent. Among them, solution polymerization is preferable.

Examples usable as the solvent in the case of producing the copolymer by the solution polymerization method may be aliphatic alcohols such as methanol, ethanol, isopropanol, and glycol; cellosolves such as cellosolve and butyl cellosolve; carbitols such as carbitol and butyl carbitol; esters such as acetic acid cellosolve, acetic acid carbitol, propylene glycol monomethyl ether acetate; ethers such as diethylene glycol dimethyl ether; cyclic ethers such as tetrahydrofuran; ketones such as cyclohexanone, methyl ethyl ketone, and methyl isobutyl ketone; and polar organic solvents such as dimethyl sulfoxide and dimethylformamide.

Examples usable as the solvent in the case of producing the copolymer by non-aqueous dispersion polymerization such as suspension polymerization, dispersion polymerization, and emulsion polymerization are liquid hydrocarbons such as benzene, toluene, hexane, and cyclohexane; and other non-polar organic solvents.

A radical polymerization initiator to be used in the case of producing the copolymer is not particularly limited and conventionally known radical polymerization initiators such as peroxides and azo initiators may be used.

The use amount of the radical polymerization initiator is not particularly limited, however, it is preferably 0.001 parts by weight in the lower limit and 5.0 parts by weight in the upper limit and more preferably 0.5 parts by weight in the lower limit and 3.0 parts by weight in the upper limit to 100 parts by weight of the entire monomer units of the copolymer.

Examples usable as the molecular weight adjustment agent may be α-methylstyrene dimer and mercaptan type chain transfer agents. Among them, long chain alkyl mercaptans having 8 or more carbon atoms are preferable in terms of little malodor and coloration.

In the curable resin composition for a column spacer of the first present invention, the content of the alkali-soluble polymer compound is not particularly limited, however, it is preferably 10% by weight in the lower limit and 80% by weight in the upper limit. If it is lower than 10% by weight, the solubility in the alkaline developer solution becomes insufficient at the time of column spacer production using the curable resin composition for a column spacer of the first present invention and accordingly the developability of the pattern of the column spacer to be produced may sometimes become insufficient, and if it exceeds 80% by weight, the curable resin composition for a column spacer of the first present invention is not sufficiently photo-cured and accordingly it may be sometimes impossible to form a pattern of the column spacer by photolithography. It is more preferably 20% by weight in the lower limit and 60% by weight in the upper limit.

The curable resin composition for a column spacer of the first present invention contains the photo-reaction initiator.

The photo-reaction initiator is not particularly limited and may be conventionally known photo-reaction initiators such as benzoin, benzophenone, benzyl, thioxanthone and derivatives of these. Practical examples are benzoin methyl ether, benzoin ethyl ether, benzoin isobutyl ether, Michler's ketone, (4-(methylphenylthio)phenyl)phenylmethanone, 2,2-dimethoxy-1,2-diphenylethan-1-one, 1-hydroxy-cyclohexyl phenyl ketone, 2-hydroxy-2-methyl-1-phenyl-propane-1-one, 1-(4-(2-hydroxyethoxy)-phenyl)-2-hydroxy-2-methyl-1-propane-1-one, 2-methyl-1(4-methylthio)phenyl)-2-morpholinopropane-1-one, 2-benzyl-2-dimethylamino-1-(4-morpholinophenyl)-butanone-1, bis(2,4,6-trimethylbenzoyl)-phenylphosphine oxide, bis(2,6-dimethoxybenzoyl)-2,4,4-trimethyl-pentylphosphine oxide, 2,4,6-trimethylbenzoyl-diphenyl-phosphine oxide, 2,4-diethylthioxanthone, and 2-chlorothioxanthone.

Further, preferably usable examples may include 2-(4-methylbenzyl)-2-(dimethylamino)-1-(4-morpholinophenyl)butane-1-one, 2-(4-ethylbenzyl)-2-(dimethylamino)-1-(4-morpholinophenyl)butane-1-one, and 2-(4-isopropylbenzyl)-2-(dimethylamino)-1-(4-morpholinophenyl)butane-1-one. Commercialized products of these compounds are “Irgacure 369” and “Irgacure 379” (manufactured by Ciba Specialty Chemicals Inc.).

Further, usable examples may include oligo[2-hydroxy-2-methyl-1-[4-(1-methylvinyl)phenyl]propanone], 1-[4-(4-benzoylphenylsulfanyl)phenyl]-2-methyl-2-(4-methylphenylsulfinyl)propane-1-one, 2,4-diethylthioxanthone, isopropylthioxanthone, diphenyl-(2,4,6-trimethylbenzoyl)phosphine oxide, 4-benzoyl-4′-methyldiphenyl sulfide, methylbenzoyl formate, 4-phenylbenzophenone, ethyl-4-(dimethylamino)benzoate, benzyl dimethyl ketal, 2-hydroxy-2-methyl-1-phenyl-1-propanone, hydroxycyclohexyl phenyl ketone, 2-methyl-1-[4-(methylthio)-phenyl]-2-morpholinopropan-1-one, methyl-2-benzoyl benzoate, 4-methylbenzophenone, 2,2′-bis-(2-chlorophenyl)-4,5,4′,5′-tetraphenyl-2′H-<1,2′-biimidazoyl, (4,4′-bis(diethylamino)benzophenone, 2,2′-bis(o-700phenyl)-4,4′,5,5′-tetraphenyl-1,2′-biimidazole; and 1-[9-ethyl-6-benzoyl-9.H.-carbazol-3-yl]-octane-1-oneoxime-O-acetate, 1-[9-ethyl-6-(2-methylbenzoyl)-9.H.-carbazol-3-yl]-ethane-1-oneoxime-O-benzoate, 1-[9-ethyl-6-(2-methylbenzoyl)-9.H.-carbazol-3-yl]-ethane-1-oneoxime-O-acetate, 1-[9-ethyl-6-(0,1,3,5-trimethylbenzoyl)-9.H.-carbazol-3-yl]-ethane-1-oneoxime-O-benzoate, and 1-[9-n-butyl-6-(2-ethylbenzoyl)-9.H.-carbazol-3-yl]-ethane-1-oneoxime-O-benzoate. Among them, oxime ester compounds such as 1-[9-ethyl-6-(2-methylbenzoyl)-9.H.-carbazol-3-yl]-ethane-1-oneoxime-O-acetate are preferably used, and commercialized products of such oxime ester compounds is, for example, “Irgacure OXE02” (manufactured by Ciba Specialty Chemicals Inc.).

These photo-reaction initiators may be used alone or two or more of them may be used in combination.

In the curable resin composition for a column spacer of the first present invention, the content of the photo-reaction initiator is not particularly limited, however, it is preferably 1% by weight in the lower limit and 20% by weight in the upper limit. If it is lower than 1% by weight, the curable resin composition for a column spacer of the first present invention may not be sometimes photo-cured and if it exceeds 20% by weight, alkali development cannot be sometimes carried out by photolithography. It is more preferably 5% by weight in the lower limit and 15% by weight in the upper limit.

The curable resin composition for a column spacer of the second present invention is a curable resin composition for a column spacer, which contains a compound having two or more polymerizable unsaturated bonds in a molecule, an alkali-soluble polymer compound, and a photo-reaction initiator, the compound having two or more polymerizable unsaturated bonds in a molecule being an oxide-modified compound having one or more hydroxyl group and two or more polymerizable unsaturated bonds in a molecule.

In the curable resin composition for a column spacer of the second present invention, the compound having two or more polymerizable unsaturated bonds in a molecule is an oxide-modified compound having one or more hydroxyl group and two or more polymerizable unsaturated bonds in a molecule. The curable resin composition for a column spacer of the second present invention containing such an oxide-modified compound having one or more hydroxyl group and two or more polymerizable unsaturated bonds in a molecule (hereinafter, referred to also as a polymerizable compound according to the second present invention) is excellent in the recovery property of the column spacer obtained by using the curable resin composition for a column spacer from the compressive deformation, and is capable of simultaneously suppressing “gravity defect” due to liquid crystal expansion at the time of heating and “cold bubble” due to contraction of the liquid crystal at the time of low temperature in a liquid crystal display panel produced by using the column spacer. Further, at the time of pattern formation, the developability and the solubility can be improved when being used for a column spacer and sharp resolution can be obtained while suppressing generation of development residues.

Non-limiting examples of the polymerizable compound according to the second present invention are oxide-modified polyfunctional (meth)acrylate compounds having one or more hydroxyl group in a molecule (hereinafter, referred to also as a polyfunctional (meth)acrylate according to the second present invention).

Examples of the polyfunctional (meth)acrylate according to the second present invention are compounds obtained by oxide-modifying difunctional (meth)acrylate compounds such as trimethylolpropane di(meth)acrylate, trimethylolethane di(meth)acrylate, pentaerythritol di(meth)acrylate, ditrimethylolpropane di(meth)acrylate, and dipentaerythritol di(meth)acrylate; and compounds obtained by oxide-modifying tri- or higher-functional (meth)acrylate compounds such as pentaerythritol tri(meth)acrylate, ditrimethylolpropane tri(meth)acrylate, dipentaerythritol tetra(meth)acrylate, and dipentaerythritol penta(meth)acrylate. Among them, compounds obtained by oxide-modifying tri- or higher-functional (meth)acrylate compounds are preferable since the polymerization reaction of these compounds proceeds quickly and the exposure sensitivity is easy to be improved. These polyfunctional (meth)acrylate compounds according to the second present invention may be used alone or two or more of them may be used in combination.

The modification degree of the oxide-modification of the polyfunctional (meth)acrylate according to the second present invention is preferably 0.5 n mole in the lower limit and 10 n mole in the upper limit to 1 mole of the polyfunctional (meth)acrylate compound, wherein n means the number of the functional groups of the polyfunctional (meth)acrylate compound as the base. If it is lower than 0.5 n mole, the resolution and solubility may sometimes become insufficient at the time of development and if it exceeds 10 n mole, the affinity with an alkaline developer solution increases and resolution tends to easily decrease due to swelling. It is more preferably 1 n mole in the lower limit and 5 n mole in the upper limit.

In the curable resin composition for a column spacer of the second present invention, the polymerizable compound according to the second present invention may be obtained preferably by a method involving synthesizing an oxide-modified alcohol by reaction of a tri- or higher-hydric alcohol and an oxide and successively esterifying the oxide-modified alcohol and (meth)acrylic acid at a proper ratio to form two or more polymerizable unsaturated bonds with simultaneously leaving a hydroxyl group; a method involving synthesizing an oxide-modified alcohol by reaction of a tri- or higher-hydric alcohol and an oxide, successively esterifying the oxide-modified alcohol and (meth)acrylic acid to obtain an oxide-modified compound having three or more polymerizable unsaturated bonds in a molecule, and successively causing reaction of the obtained compound with a compound having a hydroxyl group and a primary or secondary amino group at a proper ratio to leave two or more polymerizable unsaturated bonds and simultaneously introduce a hydroxyl group.

Non-limiting examples of the oxide-modified compound having three or more polymerizable unsaturated bonds in a molecule are compounds obtained by oxide-modifying pentaerythritol tetra(meth)acrylate, ditrimethylolpropane tetra(meth)acrylate, and dipentaerythritol hexa(meth)acrylate.

Non-limiting examples of the compound having a hydroxyl group and a primary or secondary amino group are monoethanolamine, n-propanolamine, isopropanolamine, diethanolamine, and diisopropanolamine.

In the case the polymerizable compound according to the second present invention is produced by reaction of the compound having a hydroxyl group and a primary or secondary amino group with the oxide-modified compound having three or more polymerizable unsaturated bonds in a molecule, the amino group of the compound having a hydroxyl group and a primary or secondary amino group is added to the unsaturated double bond portions of the oxide-modified compound having three or more polymerizable unsaturated bonds in a molecule by so-called Michel addition reaction.

In the Michel addition reaction of the oxide-modified compound having three or more polymerizable unsaturated bonds in a molecule and the compound having a hydroxyl group and a primary or secondary amino group, a method preferable to be employed involves dropping slowly the compound having hydroxyl group and a primary or secondary amino group without a solvent or diluted with a solvent to the oxide-modified compound having three or more polymerizable unsaturated bonds in a molecule while stirring these compounds.

The solvent to dilute the compound having a hydroxyl group and a primary or secondary amino group is not particularly limited and solvents which are not reacted with the compound having a hydroxyl group and a primary or secondary amino group and are compatible with the oxide-modified compound having three or more polymerizable unsaturated bonds in a molecule and the compound having a hydroxyl group and a primary or secondary amino group are properly selected. The solvent is preferably a water-soluble solvent having a boiling point of 64 to 200° C.

At the time of dropping into the oxide-modified compound having three or more polymerizable unsaturated bonds in a molecule, the concentration of the compound having a hydroxyl group and a primary or secondary amino group in the solvent is not particularly limited, however, it is preferably 5% by weight in the lower limit and 30% by weight in the upper limit, and more preferably 10% by weight in the lower limit and 20% by weight in the upper limit.

The Michel addition reaction proceeds quickly under normal temperature and no catalyst condition, however, if necessary, the reaction may be carried out using a catalyst or under heating condition in a range from a normal temperature to about 80° C.

The catalyst is not particularly limited and examples are alcoholates of alkali metals, organometal compounds of such as tin and titanium, metal hydroxides, and tertiary amines.

The reaction time of the Michel addition reaction is not particularly limited, however, it is preferably 1 hour in the lower limit and 10 hours in the upper limit and more preferably 3 hours in the lower limit and 7 hours in the upper limit.

The reaction solvent to be used for the Michel addition reaction is not particularly limited, however, the solvent is preferably a water-soluble solvent which does not react on the oxide-modified compound having three or more polymerizable unsaturated bonds in a molecule and the compound having a hydroxyl group and a primary or secondary amino group and is capable of dissolving their raw materials.

Practical examples are methyl alcohol, ethyl alcohol, propyl alcohol, isopropyl alcohol, tert-butyl alcohol, N-methylpyrrolidone, ε-caprolactam, ethylene glycol, ethylene glycol monomethyl ether, ethylene glycol monoethyl ether, ethylene glycol monoacetate, ethylene glycol monomethyl ether acetate, 2-(methoxymethoxy)ethanol, 2-isopropoxyethanol, 2-isopentyloxyethanol, 2-butoxyethanol, furfuryl alcohol, tetrahydrofurfuryl alcohol, tetrahydrofuran, diethylene glycol monomethyl ether, diethylene glycol monoethyl ether, diethylene glycol monobutyl ether, triethylene glycol, triethylene glycol monomethyl ether, propylene glycol monomethyl ether, propylene glycol monoethyl ether, dipropylene glycol monomethyl ether, dipropylene glycol monoethyl ether, glycerin ethers, glycerin monoacetate, diethylene glycol dimethyl ether, diethylene glycol diethyl ether, tetrahydropyran, trioxane, dioxane, 1,2,6-hexanetriol, 2-methyl-2,4-pentanediol, 2-butene-1,4-diol, 2,3-butanediol, 1,3-butanediol, 1,3-propanediol, 1,2-propanediol, propargyl alcohol, N,N-dimethylethanolamine, N,N-diethylethanolamine, N-ethylmorpholine, methyl lactate, and ethyl lactate.

In the Michel addition reaction, it is preferable to use a polymerization inhibitor.

Non-limiting examples of the polymerization inhibitor may be conventionally known polymerization inhibitors such as quinone derivatives such as hydroquinone, methylhydroquinone, and p-benzoquinone; and phenol derivatives such as 2,6-di-tert-butyl-p-cresol.

The amount of the hydroxyl group of the polymerizable compound according to the second present invention is not particularly limited, however, it is preferably 5 mgKOH/g in the lower limit and 200 mgKOH/g in the upper limit. If it is lower than 5 mgKOH/g, the developability of the curable resin composition for a column spacer of the second present invention may sometimes become insufficient and if it exceeds 200 mgKOH/g, a problem of gelation tends to be caused easily. It is more preferably 10 mgKOH/g in the lower limit and 50 mgKOH/g in the upper limit.

In the curable resin composition for a column spacer of the second present invention, the content of the polymerizable compound according to the second present invention is not particularly limited, however, it is preferably 20% by weight in the lower Limit and 90% by weight in the upper limit to the solid matter of the curable resin composition for a column spacer of the second present invention. If it is lower than 20% by weight, the curable resin composition for a column spacer of the second present invention is not sufficiently photo-cured and accordingly it may be sometimes impossible to form a pattern of the column spacer by photolithography and if it exceeds 90% by weight, the solubility in an alkaline developer solution becomes insufficient at the time of column spacer production using the curable resin composition for a column spacer of the second present invention and accordingly the developability of the pattern of the column spacer to be produced may sometimes become insufficient. It is more preferably 40% by weight in the lower limit and 80% by weight in the upper limit.

Same as the curable resin composition for a column spacer of the first present invention, the curable resin composition for a column spacer of the second present invention may contain a polymerizable unsaturated bond-containing compound in addition to the polymerizable compound according to the second present invention.

The curable resin composition for a column spacer of the second present invention contains an alkali-soluble polymer compound.

Examples of the alkali-soluble polymer compound are same as the compounds exemplified as the alkali-soluble polymer compound in the curable resin composition for a column spacer of the first present invention.

In the curable resin composition for a column spacer of the second present invention, the content of the alkali-soluble polymer compound is not particularly limited, however, it is preferably 10% by weight in the lower limit and 80% by weight in the upper limit. If it is lower than 10% by weight, the solubility in an alkaline developer solution becomes insufficient at the time of column spacer production using the curable resin composition for a column spacer of the second present invention and accordingly the developability of the pattern of the column spacer to be produced may sometimes become insufficient, and if it exceeds 80% by weight, the curable resin composition for a column spacer of the second present invention is not sufficiently photo-cured and accordingly it may be sometimes impossible to form a pattern of the column spacer by photolithography. It is more preferably 20% by weight in the lower limit and 60% by weight in the upper limit.

The curable resin composition for a column spacer of the second present invention contains a photo-reaction initiator.

Examples of the photo-reaction initiator are same as those exemplified as the photo-reaction initiator of the curable resin composition for a column spacer of the first present invention.

In the curable resin composition for a column spacer of the second present invention, the content of the photo-reaction initiator is not particularly limited, however, it is preferably 1% by weight in the lower limit and 20% by weight in the upper limit. If it is lower than 1% by weight, the curable resin composition for a column spacer of the second present invention may not be sometimes photo-cured, and if it exceeds 20% by weight, alkali development may be sometimes impossible in photolithography. It is more preferably 5% by weight in the lower limit and 15% by weight in the upper limit.

The curable resin composition for a column spacer of the third present invention is a curable resin composition for a column spacer, which contains a compound having two or more polymerizable unsaturated bonds in a molecule, an alkali-soluble polymer compound, and a photo-reaction initiator, the compound having two or more polymerizable unsaturated bonds in a molecule being a lactone-modified and oxide-modified compound having two or more polymerizable unsaturated bonds in a molecule.

In the curable resin composition for a column spacer of the third present invention, the compound having two or more polymerizable unsaturated bonds in a molecule is a lactone-modified and oxide-modified compound having two or more polymerizable unsaturated bonds in a molecule.

The curable resin composition for a column spacer of the third present invention containing such a lactone-modified and oxide-modified compound having two or more polymerizable unsaturated bonds in a molecule (hereinafter, referred to also as a polymerizable compound according to the third present invention) is excellent in the recovery property of the column spacer obtained by using the curable resin composition for a column spacer from the compressive deformation and is capable of simultaneously suppressing “gravity defect” due to liquid crystal expansion at the time of heating and “cold bubble” due to contraction of the liquid crystal at the time of low temperature in a liquid crystal display panel produced by using the column spacer. Further, at the time of pattern formation to form a column spacer by the photolithographic technique, sharp resolution can be obtained without leaving development residues.

The polymerizable compound according to the third present invention is not particularly limited, however, the compound is preferably, for example, a lactone-modified and oxide-modified polyfunctional (meth)acrylate compound (hereinafter polyfunctional (meth)acrylate according to the third present invention).

In this description, lactone-modification means introduction of a ring-opened body or ring-opened polymer of a lactone between the alcohol-derived portion and a (meth)acryloyl group of the (meth)acrylate compound in the case the polymerizable compound according to the third present invention is the polyfunctional (meth)acrylate according to the third present invention.

The lactone is not particularly limited, however, caprolactone is used preferably. Non-limiting examples of the caprolactone are ε-caprolactone, δ-caprolactone, and γ-caprolactone and among them, ε-caprolactone is preferable.

Further, non-limiting examples of the lactone other than caprolactone are δ-valerolactone, γ-butyrolactone, γ-valerolactone, and β-propiolactone. These lactones may be used alone or two or more of them may be used in combination.

Non-limiting examples of the polyfunctional (meth)acrylate according to third present invention are compounds obtained by lactone-modifying and oxide-modifying difunctional (meth)acrylate compounds and tri- or higher-functional (meth)acrylate compounds as described in the curable resin composition for a column spacer of the first present invention. Among them, the lactone-modifying and oxide-modifying tri- or higher-functional (meth)acrylate compounds are preferable since the polymerization reaction of these compounds proceeds quickly and the exposure sensitivity is easy to be improved.

The polyfunctional (meth)acrylate according to the third present invention may be used alone or two or more of them may be used in combination.

The modification degree of the lactone-modification of polyfunctional (meth)acrylate according to the third present invention is preferably 0.5 n mole in the lower limit and 5 n mole in the upper limit to 1 mole of the compound having two or more polymerizable unsaturated bonds in a molecule, wherein n means the number of the functional groups of the polyfunctional (meth)acrylate compound as the base. If it is lower than 0.5 n mole, the flexibility of the column spacer to be produced may sometimes become insufficient and if it exceeds 5 n mole, the reactivity decreases at the time of exposure in the column spacer production and patterning of the column spacer to be produced may sometimes become difficult. It is more preferably 1 n mole in the lower limit and 3 n mole in the upper limit.

The modification degree of the oxide-modification of the polyfunctional (meth)acrylate according to the third present invention is preferably 0.5 n mole in the lower limit and 4 n mole in the upper limit to 1 mole of the polyfunctional (meth)acrylate compound, wherein n means the number of the functional groups of the polyfunctional (meth)acrylate compound as the base. If it is lower than 0.5 n mole, the resolution and solubility may sometimes become insufficient at the time of development and if it exceeds 5 n mole, the reactivity decreases at the time of exposure in the column spacer production and patterning of the column spacer to be produced may sometimes become difficult. It is more preferably 1 n mole in the lower limit and 3 n mole in the upper limit.

Non-limiting examples of a practical method for synthesizing the polyfunctional (meth)acrylate according to the third present invention by lactone-modifying and oxide-modifying the polyfunctional (meth)acrylate compound are a method involving causing reaction of a lactone and an oxide with a polyhydric alcohol to synthesize a lactone-modified and oxide-modified alcohol, and esterifying the lactone-modified and oxide-modified alcohol with (meth)acrylic acid; and a method involving causing reaction of a (meth)acrylic acid and a lactone to synthesize lactone-modified (meth)acrylic acid, and esterifying the obtained lactone-modified (meth)acrylic acid with an oxide-modified polyhydric alcohol obtained by reaction of a polyhydric alcohol and an oxide.

With respect to the curable resin composition for a column spacer of the third present invention, the content of the polymerizable compound according to the third present invention is not particularly limited, however it is preferably 20% by weight in the lower limit and 90% by weight in the upper limit to the solid matter of the curable resin composition for a column spacer of the third present invention. If it is lower than 20% by weight, the curable resin composition for a column spacer of the third present invention is not sufficiently photo-cured and accordingly it may be sometimes impossible to form a pattern of the column spacer by photolithography and if it exceeds 90% by weight, the solubility in an alkaline developer solution becomes insufficient at the time of column spacer production using the curable resin composition for a column spacer of the third present invention and accordingly the developability of the pattern of the column spacer to be produced may sometimes become insufficient. It is more preferably 40% by weight in the lower limit and 80% by weight in the upper limit.

Same as the curable resin composition for a column spacer of the first present invention, the curable resin composition for a column spacer of the third present invention may contain a polymerizable unsaturated bond-containing compound in addition to the polymerizable compound according to the third present invention.

The curable resin composition for a column spacer of the third present invention contains an alkali-soluble polymer compound.

Examples of the alkali-soluble polymer compound are same as the compounds exemplified as the alkali-soluble polymer compound in the curable resin composition for a column spacer of the first present invention.

In the curable resin composition for a column spacer of the third present invention, the content of the alkali-soluble polymer compound is not particularly limited, however, it is preferably 10% by weight in the lower limit and 80% by weight in the upper limit. If it is lower than 10% by weight, the solubility in an alkaline developer solution becomes insufficient at the time of column spacer production using the curable resin composition for a column spacer of the third present invention and accordingly the developability of the pattern of the column spacer to be produced may sometimes become insufficient and if it exceeds 80% by weight, the curable resin composition for a column spacer of the first present invention is not sufficiently photo-cured and accordingly it may be sometimes impossible to form a pattern of the column spacer by photolithography. It is more preferably 20% by weight in the lower limit and 60% by weight in the upper limit.

The curable resin composition for a column spacer of the third present invention contains a photo-reaction initiator.

Examples of the photo-reaction initiator are same as those exemplified as the photo-reaction initiator of the curable resin composition for a column spacer of the first present invention.

In the curable resin composition for a column spacer of the third present invention, the content of the photo-reaction initiator is not particularly limited, however, it is preferably 1% by weight in the lower limit and 20% by weight in the upper limit. If it is lower than 1% by weight, the curable resin composition for a column spacer of the third present invention may not be sometimes photo-cured and if it exceeds 20% by weight, alkali development may be sometimes impossible in photolithography. It is more preferably 5% by weight in the lower limit and 15% by weight in the upper limit.

The curable resin composition for a column spacer of the fourth present invention is a curable resin composition for a column spacer, which contains a compound having two or more polymerizable unsaturated bonds in a molecule, an alkali-soluble polymer compound, and a photo-reaction initiator, the compound having two or more polymerizable unsaturated bonds in a molecule being a lactone-modified compound having one or more hydroxyl group and two or more polymerizable unsaturated bonds in a molecule.

In the curable resin composition for a column spacer of the fourth present invention, the compound having two or more polymerizable unsaturated bonds in a molecule is a lactone-modified compound having one or more hydroxyl group and two or more polymerizable unsaturated bonds in a molecule.

The curable resin composition for a column spacer of the fourth present invention containing such a lactone-modified compound having one or more hydroxyl group and two or more polymerizable unsaturated bonds in a molecule (hereinafter, referred to also as a polymerizable compound according to the fourth present invention) is capable of simultaneously suppressing “gravity defect” due to liquid crystal expansion at the time of heating and “cold bubble” due to contraction of the liquid crystal at the time of low temperature in a liquid crystal display panel produced by using the column spacer. Further, at the time of pattern formation to form the column spacer by the photolithographic technique, sharp resolution can be obtained without leaving development residues.

The polymerizable compound according to the fourth present invention is not particularly limited, however, the lactone-modified polyfunctional (meth)acrylate compound having one or more hydroxyl group in a molecule (hereinafter, referred to also as a polyfunctional (meth)acrylate according to the fourth present invention) is preferable.

Non-limiting examples of the polyfunctional (meth)acrylate according to the fourth present invention are compounds obtained by lactone-modifying difunctional (meth)acrylate compounds such as trimethylolpropane di(meth)acrylate, trimethylolethane di(meth)acrylate, pentaerythritol di(meth)acrylate, ditrimethylolpropane di(meth)acrylate, and dipentaerythritol di(meth)acrylate; and compounds obtained by lactone-modifying tri- or higher-functional (meth)acrylate compounds such as pentaerythritol tri(meth)acrylate, pentaerythritol tri(meth)acrylate, ditrimethylolpropane tri(meth)acrylate, dipentaerythritol tetra(meth)acrylate, and dipentaerythritol penta(meth)acrylate. Among them, compounds obtained by lactone-modifying tri- or higher-functional (meth)acrylate compounds are preferable in particular since the polymerization reaction of these compounds proceeds quickly and the exposure sensitivity is easy to be improved.

These polyfunctional (meth)acrylates according to the fourth present invention may be used alone or two or more of them may be used in combination.

The modification degree of the lactone-modification of the polyfunctional (meth)acrylate according to the fourth present invention is preferably 0.5 n mole in the lower limit and 5 n mole in the upper limit to 1 mole of the compound having two or more polymerizable unsaturated bonds in a molecule, wherein n means the number of the functional groups of the polyfunctional (meth)acrylate compound as the base. If it is lower than 0.5 n mole, the flexibility of the column spacer to be produced may sometimes become insufficient and if it exceeds 5 n mole, the reactivity decreases at the time of exposure in the column spacer production and patterning of the column spacer to be produced may sometimes become difficult. It is more preferably 1 n mole in the lower limit and 3 n mole in the upper limit.

The polymerizable compound according to the fourth present invention is obtained preferably by a method involving synthesizing a lactone-modified alcohol by reaction of a tri- or higher-hydric alcohol and a lactone and successively esterifying the lactone-modified alcohol and (meth)acrylic acid at a proper ratio to form two or more polymerizable unsaturated bonds with simultaneously leaving a hydroxyl group; a method involving synthesizing a lactone-modified (meth)acrylic acid by reaction of (meth)acrylic acid and a lactone and successively esterifying the lactone-modified (meth)acrylic acid and a tri- or higher-hydric alcohol at a proper ratio to form two or more polymerizable unsaturated bonds with simultaneously leaving a hydroxyl group; a method involving adding (meth)acrylic acid to a tri- or higher-hydric alcohol and a lactone at a proper ratio to form two or more polymerizable unsaturated bonds with simultaneously leaving a hydroxyl group and carrying out reaction collectively; and a method involving synthesizing a lactone-modified alcohol by reaction of a tri- or higher-hydric alcohol and a lactone, successively esterifying the lactone-modified alcohol and (meth)acrylic acid to obtain a lactone-modified compound having three or more polymerizable unsaturated bonds in a molecule, and successively causing reaction of the obtained compound with a compound having a hydroxyl group and a primary or secondary amino group at a proper ratio to leave two or more polymerizable unsaturated bonds and simultaneously introduce a hydroxyl group.

Non-limiting examples of the lactone-modified compound having three or more polymerizable unsaturated bonds in a molecule are compounds obtained by lactone-modifying pentaerythritol tetra(meth)acrylate and dipentaerythritol hexa(meth)acrylate.

The compound having a hydroxyl group and a primary or secondary amino group may be same as those exemplified in the polymerizable compound according to the second present invention.

In the case the polymerizable compound according to the fourth present invention is produced by reaction of the compound having a hydroxyl group and a primary or secondary amino group with the lactone-modified compound having three or more polymerizable unsaturated bonds in a molecule, the amino group of the compound having a hydroxyl group and a primary or secondary amino group is added to the unsaturated double bond portions of the lactone-modified compound having three or more polymerizable unsaturated bonds in a molecule by so-called Michel addition reaction.

The method and conditions of the Michel addition reaction are same method and conditions as the Michel addition reaction described in the polymerizable compound according to the second present invention.

The amount of the hydroxyl group of the polymerizable compound according to the fourth present invention is not particularly limited, however, it is preferably 5 mgKOH/g in the lower limit and 200 mgKOH/g in the upper limit. If it is lower than 5 mgKOH/g, the developability of the curable resin composition for a column spacer of the fourth present invention may sometimes become insufficient and if it exceeds 200 mgKOH/g, a problem of gelation tends to be caused easily. It is more preferably 10 mgKOH/g in the lower limit and 50 mgKOH/g in the upper limit.

In the curable resin composition for a column spacer of the fourth present invention, the content of the polymerizable compound according to the fourth present invention is not particularly limited, however, it is preferably 20% by weight in the lower limit and 90% by weight in the upper limit to the solid matter of the curable resin composition for a column spacer of the fourth present invention. If it is lower than 20% by weight, the curable resin composition for a column spacer of the fourth present invention is not sufficiently photo-cured and accordingly it may be sometimes impossible to form the pattern of the column spacer by photolithography and if it exceeds 90% by weight, the solubility in an alkaline developer solution becomes insufficient at the time of column spacer production using the curable resin composition for a column spacer of the fourth present invention and accordingly the developability of the pattern of the column spacer to be produced may sometimes become insufficient. It is more preferably 40% by weight in the lower limit and 80% by weight in the upper limit.

Same as the curable resin composition for a column spacer of the first present invention, the curable resin composition for a column spacer of the fourth present invention may contain a polymerizable unsaturated bond-containing compound in addition to the polymerizable compound according to the fourth present invention.

The curable resin composition for a column spacer of the fourth present invention contains an alkali-soluble polymer compound.

Examples of the alkali-soluble polymer compound are same as the compounds described as the alkali-soluble polymer compound in the curable resin composition for a column spacer of the first present invention.

In the curable resin composition for a column spacer of the fourth present invention, the content of the alkali-soluble polymer compound is not particularly limited, however, it is preferably 10% by weight in the lower limit and 80% by weight in the upper limit. If it is lower than 10% by weight, the solubility in an alkaline developer solution becomes insufficient at the time of column spacer production by using the curable resin composition for a column spacer of the fourth present invention and accordingly the developability of the pattern of the column spacer to be produced may sometimes become insufficient and if it exceeds 80% by weight, the curable resin composition for a column spacer of the fourth present invention is not sufficiently photo-cured and accordingly it may be sometimes impossible to form the pattern of the column spacer by photolithography. It is more preferably 20% by weight in the lower limit and 60% by weight in the upper limit.

The curable resin composition for a column spacer of the fourth present invention contains a photo-reaction initiator.

Examples of the photo-reaction initiator are same as those described as the photo-reaction initiator of the curable resin composition for a column spacer of the first present invention.

In the curable resin composition for a column spacer of the fourth present invention, the content of the photo-reaction initiator is not particularly limited, however, it is preferably 1% by weight in the lower limit and 20% by weight in the upper limit. If it is lower than 1% by weight, the curable resin composition for a column spacer of the fourth present invention may not be sometimes sufficiently photo-cured and if it exceeds 20% by weight, alkali development may be sometimes impossible in photolithography. It is more preferably 5% by weight in the lower limit and 15% by weight in the upper limit.

The curable resin composition for a column spacer of the fifth present invention is curable resin composition for a column spacer, which contains a compound having two or more polymerizable unsaturated bonds in a molecule, an alkali-soluble polymer compound, and a photo-reaction initiator, the compound having two or more polymerizable unsaturated bonds in a molecule being a lactone-modified and oxide-modified compound having one or more hydroxyl group and two or more polymerizable unsaturated bonds in a molecule.

In the curable resin composition for a column spacer of the fifth present invention, the compound having two or more polymerizable unsaturated bonds in a molecule is a lactone-modified and oxide-modified compound having one or more hydroxyl group and two or more polymerizable unsaturated bonds in a molecule.

The curable resin composition for a column spacer of the fifth present invention containing such a lactone-modified and oxide-modified compound having one or more hydroxyl group and two or more polymerizable unsaturated bonds in a molecule (hereinafter, referred to also as a polymerizable compound according to the fifth present invention) is capable of more preferably simultaneously suppressing “gravity defect” due to liquid crystal expansion at the time of heating and “cold bubble” due to contraction of the liquid crystal at the time of low temperature in using the column spacer production. Further, at the time of pattern formation to form the column spacer by the photolithographic technique, a sharp resolution can be obtained without leaving development residues.

The polymerizable compound according to the fifth present invention is not particularly limited, however, the compound is preferably a lactone-modified and oxide-modified polyfunctional (meth)acrylate compound having one or more hydroxyl group and two or more polymerizable unsaturated bonds in a molecule (hereinafter, referred to also as a polyfunctional (meth)acrylate according to the fifth present invention).

Non-limiting examples of the polyfunctional (meth)acrylate according to the fifth present invention are compounds obtained by lactone-modifying and oxide-modifying difunctional (meth)acrylate compounds such as trimethylolpropane di(meth)acrylate, trimethylolethane di(meth)acrylate, pentaerythritol di(meth)acrylate, ditrimethylolpropane di(meth)acrylate, and dipentaerythritol di(meth)acrylate; and compounds obtained by lactone-modifying and oxide-modifying tri- or higher-functional (meth)acrylate compounds such as pentaerythritol tri(meth)acrylate, pentaerythritol tri(meth)acrylate, ditrimethylolpropane tri(meth)acrylate, dipentaerythritol tetra(meth)acrylate, and dipentaerythritol penta(meth)acrylate. Among them, compounds obtained by lactone-modifying and oxide-modifying tri- or higher-functional (meth)acrylate compounds are preferable since the polymerization reaction of these compounds proceeds quickly and the exposure sensitivity is easy to be improved.

These polyfunctional (meth)acrylates according to the fifth present invention may be used alone or two or more of them may be used in combination.

The modification degree of the lactone-modification of the polyfunctional (meth)acrylate according to the fifth present invention is preferably 0.5 n mole in the lower limit and 5 n mole in the upper limit to 1 mole of the compound having two or more polymerizable unsaturated bonds in a molecule, wherein n means the number of the functional groups of the polyfunctional (meth)acrylate compound as the base. If it is lower than 0.5 n mole, the flexibility of the column spacer to be produced may sometimes become insufficient and if it exceeds 5 n mole, the reactivity decreases at the time of exposure in the column spacer production and patterning of the column spacer to be produced may sometimes become difficult. It is more preferably 1 n mole in the lower limit and 3 n mole in the upper limit.

The modification degree of the oxide-modification of the polyfunctional (meth)acrylate according to the fifth present invention is preferably 0.5 n mole in the lower limit and 4 n mole in the upper limit to 1 mole of the polyfunctional (meth)acrylate compound, wherein n means the number of the functional groups of the polyfunctional (meth)acrylate compound as the base. If it is lower than 0.5 n mole, the resolution and solubility may sometimes become insufficient at the time of development and if it exceeds 5 n mole, the reactivity decreases at the time of column spacer production and accordingly the patterning of the column spacer to be produced may sometimes become difficult. It is more preferably 1 n mole in the lower limit and 3 n mole in the upper limit.

The polymerizable compound according to the fifth present invention may be obtained by a method involving causing reaction of lactone and oxide with a tri- or higher-hydric alcohol to synthesize a lactone-modified and oxide-modified alcohol and esterifying the lactone-modified and oxide-modified alcohol with (meth)acrylic acid at a proper ratio to form two or more polymerizable unsaturated bonds with simultaneously leaving a hydroxyl group; a method involving causing reaction of (meth)acrylic acid and a lactone to synthesize lactone-modified (meth)acrylic acid and esterifying an oxide-modified alcohol obtained by reaction of tri- or higher-hydric alcohol and an oxide and the lactone-modified (meth)acrylic acid at a proper ratio to form two or more polymerizable unsaturated bonds with simultaneously leaving a hydroxyl group; a method involving causing reaction of a tri- or higher-hydric alcohol with a lactone and an oxide to synthesize a lactone-modified and oxide-modified alcohol and successively esterifying the lactone-modified and oxide-modified alcohol and (meth)acrylic acid to obtain a compound having three or more polymerizable unsaturated bonds in a molecule, and successively causing reaction of the obtained compound with a compound having a hydroxyl group and a primary or secondary amino group; a method involving causing reaction of (meth)acrylic acid and a lactone to synthesize lactone-modified (meth)acrylic acid, successively causing reaction of tri- or higher-alcohol with an oxide to synthesize oxide-modified alcohol, esterifying the oxide-modified alcohol and the lactone-modified (meth)acrylic acid to obtain a compound having three or more polymerizable unsaturated bonds in a molecule, and causing reaction of the compound with a compound having a hydroxyl group and a primary or secondary amino group.

Non-limiting examples of the lactone-modified and oxide-modified compound having three or more polymerizable unsaturated bonds in a molecule may be compounds obtained by lactone-modifying and oxide-modifying pentaerythritol tetra(meth)acrylate and dipentaerythritol hexa(meth)acrylate.

The compound having a hydroxyl group and a primary or secondary amino group are same as the compounds described in the polymerizable compound according to the second present invention.

In the case the polymerizable compound according to the fifth present invention is produced by reaction of the compound having a hydroxyl group and a primary or secondary amino group with the lactone-modified and oxide-modified compound having three or more polymerizable unsaturated bonds in a molecule, the amino group of the compound having a hydroxyl group and a primary or secondary amino group is added to the unsaturated double bond portions of the lactone-modified and oxide-modified compound having three or more polymerizable unsaturated bonds in a molecule by so-called Michel addition reaction.

The method and conditions of the Michel addition reaction are same method and conditions as the Michel addition reaction described in the polymerizable compound according to the second present invention.

The amount of the hydroxyl group of the polymerizable compound according to the fifth present invention is not particularly limited, however, it is preferably 5 mgKOH/g in the lower limit and 200 mgKOH/g in the upper limit. If it is lower than 5 mgKOH/g, the developability of the curable resin composition for a column spacer of the fifth present invention may sometimes become insufficient and if it exceeds 200 mgKOH/g, a problem of gelation tends to be caused easily. It is more preferably 10 mgKOH/g in the lower limit and 50 mgKOH/g in the upper limit.

In the curable resin composition for a column spacer of the fifth present invention, the content of the polymerizable compound according to the fifth present invention is not particularly limited, however, it is preferably 20% by weight in the lower limit and 90% by weight in the upper limit to the solid matter of the curable resin composition for a column spacer of the fifth present invention. If it is lower than 20% by weight, the curable resin composition for a column spacer of the fifth present invention is not sufficiently photo-cured and accordingly it may be sometimes impossible to form the pattern of the column spacer by photolithography and if it exceeds 90% by weight, the solubility in an alkaline developer solution becomes insufficient at the time of column spacer production using the curable resin composition for a column spacer of the fifth present invention and accordingly the developability of the pattern of the column spacer to be produced may sometimes become insufficient. It is more preferably 40% by weight in the lower limit and 80% by weight in the upper limit.

Same as the curable resin composition for a column spacer of the first present invention, the curable resin composition for a column spacer of the fifth present invention may contain a polymerizable unsaturated bond-containing compound in addition to the polymerizable compound according to the fifth present invention.

The curable resin composition for a column spacer of the fifth present invention contains an alkali-soluble polymer compound.

Examples of the alkali-soluble polymer compound are same as the compounds exemplified as the alkali-soluble polymer compound in the curable resin composition for a column spacer of the first present invention.

In the curable resin composition for a column spacer of the fifth present invention, the content of the alkali-soluble polymer compound is not particularly limited, however, it is preferably 10% by weight in the lower limit and 80% by weight in the upper limit. If it is lower than 10% by weight, the solubility in an alkaline developer solution becomes insufficient at the time of column spacer production using the curable resin composition for a column spacer of the fifth present invention and accordingly the developability of the pattern of the column spacer to be produced may sometimes become insufficient and if it exceeds 80% by weight, the curable resin composition for a column spacer of the fifth present invention is not sufficiently photo-cured and accordingly it may be sometimes impossible to form the pattern of the column spacer by photolithography. It is more preferably 20% by weight in the lower limit and 60% by weight in the upper limit.

The curable resin composition for a column spacer of the fifth present invention contains a photo-reaction initiator.

Examples of the photo-reaction initiator are same as those exemplified as the photo-reaction initiator of the curable resin composition for a column spacer of the first present invention.

In the curable resin composition for a column spacer of the fifth present invention, the content of the photo-reaction initiator is not particularly limited, however, it is preferably 1% by weight in the lower limit and 20% by weight in the upper limit. If it is lower than 1% by weight, the curable resin composition for a column spacer of the fifth present invention may not be sometimes sufficiently photo-cured and if it exceeds 20% by weight, alkali development may be sometimes impossible in photolithography. It is more preferably 5% by weight in the lower limit and 15% by weight in the upper limit.

The curable resin composition for a column spacer of the sixth present invention contains a compound having two or more polymerizable unsaturated bonds in a molecule, an alkali-soluble polymer compound, and a photo-reaction initiator.

In the curable resin composition according to the present invention, the compound having two or more polymerizable unsaturated bonds in a molecule has one or more carboxyl group and two or more polymerizable unsaturated bonds in a molecule (hereinafter, referred to also as a polymerizable compound according to the sixth present invention).

The polymerizable compound according to the sixth present invention is not particularly limited, however, for example, it is preferably (meth)acrylate compounds into which a carboxylic acid is introduced (hereinafter, referred to also as a carboxyl-containing polyfunctional (meth)acrylate compound) by addition reaction of a carboxyl-containing compound to a part of the (meth)acryl group of a tri- or higher-functional (meth)acrylate compound. Since the curable resin composition for a column spacer of the sixth present invention contains such a carboxyl-containing polyfunctional (meth)acrylate compound, the curable resin composition is excellent in the high polymerization reactivity required for obtaining exposure sensitivity at the time of pattern formation by photolithographic technique and also in affinity with an alkaline developer solution required for obtaining high resolution at the time of development.

The carboxyl-modification degree to the compound having one or more carboxyl group and two or more polymerizable unsaturated bonds in a molecule is not particularly limited if it is proper for the compound to be dissolved smoothly in an alkaline developer solution, however, the acid value is preferably 5 mgKOH/g in the lower limit and 80 mgKOH/g in the upper limit and more preferably 10 mgKOH/g in the lower limit and 50 mgKOH/g in the upper limit.

Non-limiting examples of the tri- or higher functional (meth)acrylate compound are trimethylolpropane tri(meth)acrylate, trimethylolethane tri(meth)acrylate, pentaerythritol tri(meth)acrylate, pentaerythritol tetra(meth)acrylate, ditrimethylolpropane tri(meth)acrylate, ditrimethylolpropane tetra(meth)acrylate, dipentaerythritol tri(meth)acrylate, dipentaerythritol tetra(meth)acrylate, dipentaerythritol penta(meth)acrylate, and dipentaerythritol hexa(meth)acrylate. Among them, pentaerythritol tri(meth)acrylate, ditrimethylolpropane tri(meth)acrylate, dipentaerythritol tri(meth)acrylate, dipentaerythritol tetra(meth)acrylate, and dipentaerythritol penta(meth)acrylate are used preferably.

Further, tri- or higher-functional urethane (meth)acrylate, epoxy (meth)acrylate, and polyester (meth)acrylate are also preferable. Examples of these urethane (meth)acrylate and epoxy (meth)acrylate are UA-306H, UA-306T, and UA-306I (all manufactured by Kyoeisha Chemical Co., Ltd.), EB9260, EB8210, EB1290, EB1290K, EB5129, EB810, EB450, EB830, EB870, and EB1870 (all manufactured by Daicel Cytec Co., Ltd.), M-1960, M-7100, M-8030, M-8060, M-8100, M-8530, M-8560, and M-9050 (all manufactured by Toagosei Co., Ltd.).

These tri- or higher-functional (meth)acrylate compounds may be used alone or two or more of them may be used in combination.

In the curable resin composition for a column spacer of the sixth present invention, in the case the polymerizable compound according to the sixth present invention is the carboxyl-containing (meth)acrylate compound, since polymerization reaction proceeds quickly and exposure sensitivity can be improved easily, the number of the (meth)acryl groups in a molecule is preferably 3 in the lower limit. Further, in the carboxyl-containing polyfunctional (meth)acrylate compound, the number of the carboxyl group in a molecule is preferably 2 in the upper limit. If it is 3 or more, the solubility and swelling property in the developer solution is increased and in the case the curable resin composition for a column spacer of the present invention is used for a column spacer, peeling of the developed pattern and decrease of resolution due to swelling property tend to occur easily.

Non-limiting examples of the carboxyl-containing compound are compounds having a carboxyl group and a thiol group such as thiosalicylic acid, mercaptoacetic acid, mercaptosuccinic acid, and 3-mercaptopropionic acid.

A method for obtaining the carboxyl-containing polyfunctional (meth)acrylate compound is not particularly limited and examples of the method may include a method involving adding a compound having a thiol group and a carboxyl group such as thiosalicylic acid to the (meth)acryl group of the tri- or higher-functional (meth)acrylate compound by en-thiol reaction.

In the curable resin composition for a column spacer of the sixth present invention, the polymerizable compound according to the sixth present invention is preferably a lactone-modified and/or oxide-modified carboxyl-containing polyfunctional (meth)acrylate compound. It is because a cured material obtained by curing the curable resin composition for a column spacer of the sixth present invention becomes excellent in the flexibility and in the case the curable resin composition for a column spacer of the sixth present invention is used for a column spacer application, a column spacer having excellent flexibility and high compression recovery property can be obtained preferably.

In the curable resin composition for a column spacer of the sixth present invention, in the case the polymerizable compound according to the sixth present invention is a lactone-modified carboxyl-containing polyfunctional (meth)acrylate compound, the polyfunctional (meth)acrylate compound is not particularly limited and the tri- or higher-(meth)acrylate compounds can be exemplified. Among them, examples preferable to be used are compounds obtained by adding carboxyl-containing compounds to caprolactone-modified pentaerythritol tri(meth)acrylate, ditrimethylolpropane tri(meth)acrylate, dipentaerythritol tri(meth)acrylate, dipentaerythritol tetra(meth)acrylate, or dipentaerythritol penta(meth)acrylate.

The modification degree of the lactone-modification of the lactone-modified polyfunctional (meth)acrylate is preferably 0.5 n mole in the lower limit and 5 n mole in the upper limit to 1 mole of the compound having two or more polymerizable unsaturated bonds in a molecule, wherein n means the number of the functional groups of the polyfunctional (meth)acrylate compound as the base. If it is lower than 0.5 n mole, the flexibility of the column spacer to be produced may sometimes become insufficient and if it exceeds 5 n mole, the reactivity decreases at the time of exposure in the column spacer production and patterning of the column spacer to be produced may sometimes become difficult. It is more preferably 1 n mole in the lower limit and 3 n mole in the upper limit.

A practical method for lactone-modifying the polyfunctional (meth)acrylate compound is not particularly limited and may be a method involving synthesizing a lactone-modified alcohol by reaction of a polyhydric alcohol and a lactone and esterifying the synthesized lactone-modified alcohol with (meth)acrylic acid; a method involving synthesizing a lactone-modified (meth)acrylic acid by reaction of (meth)acrylic acid and a lactone and esterifying the lactone-modified (meth)acrylic acid with an alcohol; and a method involving causing reaction of a (meth)acrylic acid, a caprolactone, and a polyhydric alcohol collectively.

In the case the polymerizable compound for a column spacer of the sixth present invention is the oxide-modified carboxyl-containing polyfunctional (meth)acrylate compound, the polyfunctional (meth)acrylate compound is not particularly limited and may be the tri- or higher functional (meth)acrylate compounds. Among them, examples preferable to be used are compounds obtained by adding carboxyl-containing compounds to oxide-modified pentaerythritol tri(meth)acrylate, ditrimethylolpropane tri(meth)acrylate, dipentaerythritol tri(meth)acrylate, dipentaerythritol tetra(meth)acrylate, or dipentaerythritol penta(meth)acrylate.

The modification degree of the oxide-modification of the polyfunctional (meth)acrylate is preferably 0.5 n mole in the lower limit and 15 n mole in the upper limit to 1 mole of the polyfunctional (meth)acrylate compound, wherein n means the number of the functional groups of the polyfunctional (meth)acrylate compound as the base. If it is lower than 0.5 n mole, the flexibility of the column spacer to be produced may sometimes become insufficient and if it exceeds 15 n mole, the affinity with an alkaline developer solution increases and resolution tends to easily decrease due to swelling. It is more preferably 3 n mole in the lower limit and 10 n mole in the upper limit.

A practical method for oxide-modifying the polyfunctional (meth)acrylate compound is not particularly limited and may be a method involving synthesizing an oxide-modified alcohol by reaction of a polyhydric alcohol and an oxide and esterifying the oxide-modified alcohol with (meth)acrylic acid; a method involving synthesizing an oxide-modified (meth)acrylic acid by reaction of (meth)acrylic acid and an oxide and esterifying the oxide-modified (meth)acrylic acid with an alcohol; and a method involving causing reaction of a (meth)acrylic acid, an oxide, and a polyhydric alcohol collectively.

In the curable resin composition for a column spacer of the sixth present invention, the polymerizable compound according to the sixth present invention may have one or more hydroxyl group in a molecule. The curable resin composition for a column spacer of the sixth present invention containing the polymerizable compound according to the sixth present invention can improve the developability and solubility at the time of pattern formation, suppress development residues, and give sharp resolution when being used for a column spacer.

The polymerizable compound containing a hydroxyl group in a molecule according to the invention can be obtained, for example, by adjusting the mixing ratio and/or reaction ratio of (meth)acrylic acid to be reacted with the polyhydric alcohol at the time of producing the (meth)acrylate compound.

The polymerizable compound containing a hydroxyl group in a molecule according to the sixth present invention may be obtained by addition reaction of a carboxylic acid compound having two or more carboxyl groups and/or acid anhydride with the compound having two or more polymerizable unsaturated bonds and a hydroxyl group in a molecule.

Non-limiting examples of the compound having two or more polymerizable unsaturated bonds and a hydroxyl group in a molecule may include pentaerythritol tri(meth)acrylate, ditrimethylolpropane tri(meth)acrylate, dipentaerythritol tri(meth)acrylate, dipentaerythritol tetra(meth)acrylate, and dipentaerythritol penta(meth)acrylate, and their lactone-modified and/or oxide-modified compounds.

A method for obtaining such a compound having two or more polymerizable unsaturated bonds and a hydroxyl group is not particularly limited and may be a method for causing reaction of a (meth)acrylate compound (or a compound obtained by oxide-modifying a (meth)acrylate compound) and a polyhydric alcohol; a method for causing reaction of a compound having a hydroxyl group and a primary or secondary amino group with a compound having three or more polymerizable unsaturated bonds in a molecule or its lactone-modified and/or oxide-modified compound; and a method for causing reaction of an oxide-modified polyhydric alcohol and a (meth)acrylate compound.

Non-limiting examples of the compound having three or more polymerizable unsaturated bonds in a molecule may be pentaerythritol tetra(meth)acrylate and dipentaerythritol hexa(meth)acrylate.

Non-limiting examples of the compound having a hydroxyl group and a primary or secondary amino group may be monoethanolamine, n-propanolamine, isopropanolamine, diethanolamine, and diisopropanolamine.

In the case reaction of the compound having a hydroxyl group and a primary or secondary amino group with the compound having three or more polymerizable unsaturated bonds in a molecule is carried out, the amino group of the compound having a hydroxyl group and a primary or secondary amino group is added to the unsaturated double bond portions of the compound having three or more polymerizable unsaturated bonds in a molecule by so-called Michel addition reaction.

In the Michel addition reaction of the compound having three or more polymerizable unsaturated bonds in a molecule and the compound having a hydroxyl group and a primary or secondary amino group, a method preferable to be employed involves dropping slowly the compound having a hydroxyl group and a primary or secondary amino group without a solvent or diluted with a solvent to the compound having three or more polymerizable unsaturated bonds in a molecule while stirring these compounds.

The solvent to dilute the compound having a hydroxyl group and a primary or secondary amino group is not particularly limited and, for example, solvents which are not reacted with the compound having a hydroxyl group and a primary or secondary amino group and are compatible with the compound having three or more polymerizable unsaturated bonds in a molecule and the compound having a hydroxyl group and a primary or secondary amino group, are properly selected. The solvent is preferably a water-soluble solvent having a boiling point of 64 to 200° C.

At the time of the drop to the compound having three or more polymerizable unsaturated bonds in a molecule, the concentration of the compound having a hydroxyl group and a primary or secondary amino group in the solvent is not particularly limited, however, it is preferably 5% by weight in the lower limit and 30% by weight in the upper limit and more preferably 10% by weight in the lower limit and 20% by weight in the upper limit.

The Michel addition reaction proceeds quickly under normal temperature and no solvent condition, however, if necessary, the reaction may be carried out using a catalyst or under heating condition in a range from a normal temperature to about 80° C.

The catalyst is not particularly limited and examples are alcoholates of alkali metals, organometal compounds of such as tin and titanium, metal hydroxides, and tertiary amines.

The reaction time of the Michel addition reaction is not particularly limited, however, it is preferably 1 hour in the lower limit and 10 hours in the upper limit and more preferably 3 hours in the lower limit and 7 hours in the upper limit.

Examples of the reaction solvent to be used in the Michel addition reaction are same as those described as the reaction solvent in the curable resin composition for a column spacer of the second present invention.

In the Michel addition reaction, it is preferable to use a polymerization inhibitor and examples of the polymerization inhibitor are same as those described as the polymerization inhibitor in the curable resin composition for a column spacer of the second present invention.

The carboxylic acid compound having two or more carboxyl groups may include dicarboxylic acid compounds such as oxalic acid, maleic acid, succinic acid, tartaric acid, itaconic acid, phthalic acid, tetrahydrophthalic acid, methyltetrahydrophthalic acid, ethyltetrahydrophthalic acid, hexahydrophthalic acid, methylhexahydrophthalic acid, ethylhexahydrophthalic acid, and chlorendic acid; and tricarboxylic acid compounds such as trimellitic acid. Dicarboxylic acid compounds and tricarboxylic acid compounds are preferably usable.

Non-limiting examples of the acid anhydride may include carboxylic acid anhydrides such as oxalic anhydride, maleic anhydride, succinic anhydride, tartaric anhydride, itaconic anhydride, phthalic anhydride, tetrahydrophthalic anhydride, methyltetrahydrophthalic anhydride, ethyltetrahydrophthalic anhydride, hexahydrophthalic anhydride, methylhexahydrophthalic anhydride, ethylhexahydrophthalic anhydride, chlorendic anhydride, trimellitic anhydride, pyromellitic anhydride, benzophenonetetracarboxylic acid anhydride, and biphenyltetracarboxylic acid anhydride.

These carboxylic acid compounds having two or more carboxyl groups and/or acid anhydrides are reacted on a hydroxyl group of the compounds having two or more polymerizable unsaturated bonds and a hydroxyl group by addition reaction to give the polymerizable compounds having a carboxyl group in a molecule according to the present invention.

The addition reaction of the carboxylic acid compound having two or more carboxyl groups with the hydroxyl group of the compound having two or more polymerizable unsaturated bonds and a hydroxyl group may be, for example, a common dehydration esterification reaction.

A practical example of the method is a method involving loading a compound having two or more polymerizable unsaturated bonds and a hydroxyl group in a molecule, a carboxylic acid compound having two or more carboxyl groups, and a solvent into a reactor equipped with a stirrer, a thermometer, and a water separator, heating the mixture in the presence of an acidic catalyst, removing water produced as the reaction proceeds to the outside of the system, washing the reaction solution after completion of the reaction, separating the water-phase layer, and successively removing the solvent under decreased pressure.

The solvent in the esterification reaction for adding the carboxylic acid compound having two or more carboxyl groups to the hydroxyl group of the compound having two or more polymerizable unsaturated bonds and a hydroxyl group in a molecule is not particularly limited if the solvent makes water removal easy and is not reactive with the carboxylic acid compound having two or more carboxyl groups, the compound having two or more polymerizable unsaturated bonds and hydroxyl groups in a molecule, and the acidic catalyst, and preferable examples are aliphatic hydrocarbons such as n-hexane and n-heptane; aromatic hydrocarbons such as benzene, toluene, and xylene; and alicyclic hydrocarbons such as cyclohexane, which produce azeotropic mixture with produced water.

The acidic catalyst in the esterification reaction of the carboxylic acid compound having two or more carboxyl groups and the compound having two or more polymerizable unsaturated bonds and hydroxyl groups in a molecule may be an inorganic acid or an organic acid. Practical examples of the inorganic acid are hydrochloric acid, sulfuric acid, and phosphoric acid and practical example of the organic acid is p-toluenesulfonic acid, benzenesulfonic acid, and methanesulfonic acid. Specially, organic sulfonic acid such as p-toluenesulfonic acid is preferable due to low corrosive property. The addition amount of the acidic catalyst is preferably 0.5% by weight in the lower limit and 5% by weight in the upper limit to the entire amount of the reaction solution.

The reaction temperature of the esterification reaction of the carboxylic acid compound having two or more carboxyl groups and the compound having two or more polymerizable unsaturated bonds and a hydroxyl group in a molecule is preferably 70° C. in the lower limit and 150° C. in the upper limit. Heating at a temperature in the range makes it easy to carry out dehydration and esterification reaction. It is more preferably 80° C. in the lower limit and 120° C. in the upper limit.

In the esterification reaction of the carboxylic acid compound having two or more carboxyl groups and the compound having two or more polymerizable unsaturated bonds and a hydroxyl group in a molecule, the reaction is preferable to be carried out in the presence of a polymerization inhibitor. Examples of the polymerization inhibitor are hydroquinone, hydroquinone monomethyl ether, phenothiazine, p-benzoquinone, 2,5-dihydroxy-p-benzoquinone, 4-tert-butylcatecol, and copper salt. The use amount is, in general, preferably 0.01% by weight in the lower limit and 1% by weight in the upper limit to the entire amount of the reaction solution.

The addition reaction of the acid anhydride to the hydroxyl group of the compound having two or more polymerizable unsaturated bonds and a hydroxyl group in a molecule is common esterification reaction and the reaction temperature is preferably 60° C. in the lower limit and 150° C. in the upper limit. The reaction time is preferably 1 hour in the lower limit and 12 hours in the upper limit.

Further, in the case of addition reaction of the acid anhydride to the hydroxyl group of the compound having two or more polymerizable unsaturated bonds and a hydroxyl group in a molecule, a catalyst, for example, tertiary amines such as triethylamine; quaternary ammonium salt such as triethylbenzylammonium chloride; 2-ethyl-4-methylimidazole compounds; and phosphorus compounds such as triphenylphosphine may be used. Further, as a polymerization inhibitor, conventionally known polymerization inhibitor, for example, quinone derivatives such as hydroquinone, methylhydroquinone, and p-benzoquinone; and phenol derivatives such as 2,6-di-tert-butyl-p-cresol may be used.

The addition reaction of the acid anhydride to the hydroxyl group of the compound having two or more polymerizable unsaturated bonds and hydroxyl groups in a molecule may be carried out without a solvent or in the presence of a solvent if necessary. The solvent to be used for the reaction is not particularly limited if it does not inhibit the reaction and examples may be ketones such as methyl ethyl ketone and cyclohexanone; aromatic hydrocarbons such as toluene, xylene and tetramethylbenzene; cellosolves such as cellosolve, methyl cellosolve, and butyl cellosolve; carbitols such as carbitol, methyl carbitol, and butyl carbitol; glycol ethers such as diethylene glycol dimethyl ether, diethylene glycol diethyl ether; acetic acid esters such as ethyl acetate, butyl acetate, cellosolve acetate, butyl cellosolve acetate, carbitol acetate, butyl carbitol acetate, propylene glycol monomethyl ether acetate, and dipropylene glycol monomethyl ether acetate; aliphatic hydrocarbons such as octane and decane; and petroleum type solvents such as petroleum ethers, petroleum naphtha, hydrogenated petroleum naphtha, and solvent naphtha.

The polymerizable compound according to the sixth present invention is preferably a compound obtained by addition reaction of a carboxylic acid compound having two or more carboxyl groups and/or acid anhydride; and a lactone-modified and/or oxide-modified compound having two or more polymerizable unsaturated bonds and a hydroxyl group in a molecule.

Non-limiting examples of the lactone-modified and/or oxide-modified compound having two or more polymerizable unsaturated bonds and a hydroxyl group in a molecule are lactone-modified and/or oxide-modified compounds of pentaerythritol tri(meth)acrylate, ditrimethylolpropane tri(meth)acrylate, dipentaerythritol tri(meth)acrylate, dipentaerythritol tetra(meth)acrylate, and dipentaerythritol penta(meth)acrylate.

The method for synthesizing the lactone-modified and/or oxide-modified compound having two or more polymerizable unsaturated bonds and a hydroxyl group in a molecule may be (1) a method involving synthesizing a lactone-modified polyhydric alcohol by reaction of a polyhydric alcohol and a lactone and esterifying the lactone-modified polyhydric alcohol with (meth)acrylic acid; (2) a method involving synthesizing a lactone-modified (meth)acrylic acid by reaction of (meth)acrylic acid and a lactone and esterifying the lactone-modified (meth)acrylic acid with an alcohol; and (3) a method involving causing reaction of a (meth)acrylic acid, a lactone, and a polyhydric alcohol collectively.

In the method (1), the method for synthesizing a lactone-modified polyhydric alcohol by reaction of a polyhydric alcohol and a lactone may be a method involving loading the polyhydric alcohol and the lactone into a reactor equipped with a stirrer, a thermometer, and a condenser and heating the mixture in the presence of an acidic catalyst for carrying out reaction.

Examples as the acidic catalyst to be used for synthesizing the lactone-modified polyhydric alcohol may be preferably stannous chloride, stannous octylate, and dibutyltin dilaurate. The use amount of the catalyst is preferably 0.005% by weight in the lower limit and 0.5% by weight in the upper limit to the entire amount of the reaction solution.

With respect to the reaction condition in the case of synthesizing the lactone-modified polyhydric alcohol, the reaction temperature is preferably 80° C. in the lower limit and 200° C. in the upper limit and the reaction time is preferably 1 hour in the lower limit and 20 hours in the upper limit.

The polyhydric alcohol is not particularly limited, however, it is preferable to use at least one tri- or higher-polyhydric alcohol compound selected from the group comprising pentaerythritol, dipentaerythritol, tripentaerythritol, tetrapentaerythritol, trimethylolethane, ditrimethylolethane, trimethylolpropane, and ditrimethylolpropane.

Non-limiting examples of the lactone are ε-caprolactone, δ-caprolactone, and γ-caprolactone and specially, ε-caprolactone is preferable.

The method for esterification reaction of the lactone-modified polyhydric alcohol and (meth)acrylic acid may be a common dehydration esterification method.

Practically, the method may involve loading the lactone-modified polyhydric alcohol, (meth)acrylic acid, and a solvent into a reactor equipped with a stirrer, a thermometer, and a water separator, heating the mixture in the presence of an acidic catalyst, removing water produced as the reaction proceeds to the outside of the system, washing the reaction solution after completion of the reaction, separating the water-phase layer, and successively removing the solvent under decreased pressure.

The solvent in the esterification reaction is not particularly limited if it makes water removal easy and is not reactive with the lactone-modified polyhydric alcohol, the (meth)acrylic acid, and the acidic catalyst, and preferable examples are aliphatic hydrocarbons such as n-hexane and n-heptane, aromatic hydrocarbons such as benzene, toluene, and xylene, and alicyclic hydrocarbons such as cyclohexane, which produce an azeotropic mixture with produced water.

The acidic catalyst may be an inorganic acid or an organic acid. Practical examples of the inorganic acid are hydrochloric acid, sulfuric acid, and phosphoric acid and practical examples of the organic acid are p-toluenesulfonic acid, benzenesulfonic acid, and methanesulfonic acid. Specially, organic sulfonic acid such as p-toluenesulfonic acid is preferable due to low corrosive property. The addition amount of the acidic catalyst is preferably 0.5% by weight in the lower limit and 5% by weight in the upper limit to the entire amount of the reaction solution.

The reaction temperature of the esterification reaction is preferably 70° C. in the lower limit and 150° C. in the upper limit. Heating at a temperature in the range makes it easy to carry out dehydration and esterification reaction. It is more preferably 80° C. in the lower limit and 120° C. in the upper limit.

Further, it is common that the (meth)acrylic acid is previously added with a polymerization inhibitor, however, in the esterification reaction, it is preferable to carry out the reaction in the presence of a newly added polymerization inhibitor. Examples of the polymerization inhibitor are hydroquinone, hydroquinone monomethyl ether, phenothiazine, p-benzoquinone, 2,5-dihydroxy-p-benzoquinone, 4-tert-butylcatecol, and copper salt. The use amount is, in general, preferably 0.01% by weight in the lower limit and 1% by weight in the upper limit to the entire amount of the reaction solution.

In the method (2), the method for synthesizing a lactone-modified (meth)acrylic acid by reaction of (meth)acrylic acid and a lactone may be practically a method involving loading (meth)acrylic acid and a lactone into a reactor equipped with a stirrer, a thermometer, and a reflux condenser and heating the mixture in the presence of an acidic catalyst for carrying out reaction. After completion of the reaction, the catalyst is removed by neutralization or adsorption of the reaction solution, and, if necessary, water washing, distillation or the like is carried out.

Examples as the acidic catalyst to be used for synthesizing the lactone-modified (meth)acrylic acid may be either an inorganic acid or an organic acid and practical examples are the same acidic catalysts exemplified in the esterification reaction of the method (1). The addition amount of the catalyst is preferably 0.5% by weight in the lower limit and 5% by weight in the upper limit to the entire amount of the reaction solution and more preferably 0.8% by weight in the lower limit and 3% by weight in the upper limit.

The reaction temperature in the case of synthesizing the lactone-modified (meth)acrylic acid is preferably 60° C. in the lower limit and 120° C. in the upper limit and more preferably 70° C. in the lower limit and 100° C. in the upper limit in terms of shortening of the reaction time and polymerization prevention.

In the case of synthesizing the lactone-modified (meth)acrylic acid, it is preferable to use the solvent for making the temperature control easy during the reaction. A usable solvent is not particularly limited if it does not react on (meth)acrylic acid, the lactone, and the acidic catalyst, however, aromatic hydrocarbons such as benzene, toluene, and xylene are preferable.

It is common that the (meth)acrylic acid is previously added a polymerization inhibitor, however, in the case of synthesizing the lactone-modified (meth)acrylic acid, it is preferable to carry out the reaction in the presence of a newly added polymerization inhibitor. Examples of the polymerization inhibitor are the same polymerization inhibitors exemplified in the esterification reaction of the method (1) and the addition amount is, in general, preferably 0.01% by weight in the lower limit and 1% by weight in the upper limit to the entire amount of the reaction solution.

The method for esterification reaction of the lactone-modified (meth)acrylic acid and the alcohol may be a common dehydration esterification method.

Practically, the method may involve loading the lactone-modified (meth)acrylic acid, a polyhydric alcohol, and a solvent into a reactor equipped with a stirrer, a thermometer, and a water separator, heating the mixture in the presence of an acidic catalyst, removing water produced as the reaction proceeds to the outside of the system, washing the reaction solution after completion of the reaction, separating the water-phase layer, and successively removing the solvent under decreased pressure.

Hydroxyl group in the (meth)acrylate in the esterification reaction of the method (2) can be obtained by adjusting the loading mole ratio and reaction ratio of the lactone-modified (meth)acrylic acid to the polyhydric alcohol.

In the esterification reaction of the method (2), the mole ratio of the lactone-modified (meth)acrylic acid to the polyhydric alcohol is preferably 0.6 in the lower limit and 1.2 in the upper limit and more preferably 0.7 in the lower limit and 1.0 in the upper limit.

The solvent in the esterification reaction of the method (2) is not particularly limited if it makes water removal easy and is not reactive with the lactone-modified (meth)acrylic acid, polyhydric alcohol, and acidic catalyst, and preferable examples are aromatic hydrocarbons such as benzene, toluene, and xylene, which produce an azeotropic mixture with produced water.

The acidic catalyst in the esterification reaction of the method (2) may be an organic sulfonic acid such as p-toluenesulfonic acid. The addition amount of the acidic catalyst is preferably 0.5% by weight in the lower limit and 5% by weight in the upper limit to the entire amount of the reaction solution.

The reaction temperature in the esterification reaction of the method (2) is preferably 70° C. in the lower limit and 150° C. in the upper limit. Heating at a temperature in the range makes it easy to carry out dehydration and esterification reaction. It is more preferably 80° C. in the lower limit and 120° C. in the upper limit.

In the esterification reaction of the method (2), it is preferable to add a polymerization initiator and examples of the polymerization inhibitor are the same polymerization initiator exemplified in the esterification reaction of the method (1) and the use amount is, in general, preferably 0.01% by weight in the lower limit and 1% by weight in the upper limit to the entire amount of the reaction solution.

In the method (3), the method involving causing reaction of (meth)acrylic acid, a lactone, and a polyhydric alcohol collectively may be a common dehydration esterification method.

Practically, the method may involve collectively loading (meth)acrylic acid, a lactone, a polyhydric alcohol, and a solvent into a reactor equipped with a stirrer, a thermometer, and a water separator, heating the mixture in the presence of an acidic catalyst. Water produced as the reaction proceeds is removed to the outside of the system. Completion of the reaction is determined in accordance with the water amount. The reaction solution is washed after completion of the reaction, and after the water-phase layer is separated, the solvent is removed under decreased pressure.

The acidic catalyst in the method (3) may be an inorganic acid or an organic acid and practically the same acidic catalyst exemplified in the esterification reaction of the method (1). The addition amount of the acidic catalyst is preferably 0.5% by weight in the lower limit and 5% by weight in the upper limit to the entire amount of the reaction solution.

The reaction temperature of the method (3) is preferably 70° C. in the lower limit and 150° C. in the upper limit and more preferably 100° C. in the lower limit and 120° C. in the upper limit in terms of shortening of the reaction time and polymerization prevention.

In the method (3), it is preferable to use a solvent for making temperature control easy during the reaction. The usable solvent is not particularly limited if it is not reactive with the (meth)acrylic acid, a lactone, a polyhydric alcohol, and an acidic catalyst and preferable examples are aromatic hydrocarbons such as benzene, toluene, and xylene.

Further, it is common that the (meth)acrylic acid is previously added a polymerization inhibitor, however, in the method (3), it is preferable to carry out the reaction in the presence of a newly added polymerization inhibitor. Examples of the polymerization inhibitor are the same polymerization inhibitor exemplified in the esterification reaction of the method (1). The use amount is, in general, preferably 0.01% by weight in the lower limit and 1% by weight in the upper limit to the entire amount of the reaction solution.

The method for synthesizing the oxide-modified compound having two or more polymerizable unsaturated bonds and a hydroxyl group in a molecule is not particularly limited and an example may be a method (4) involving synthesizing the oxide-modified polyhydric alcohol by reaction of a polyhydric alcohol and an oxide and esterifying the oxide-modified polyhydric alcohol and (meth)acrylic acid.

In the method (4), the method for synthesizing the oxide-modified polyhydric alcohol by reaction of the polyhydric alcohol and an oxide may be a method involving loading the polyhydric alcohol and a basic catalyst into an autoclave equipped with a stirrer, applying pressure with nitrogen, successively heating the autoclave, and carrying out reaction while introducing the oxide. After completion of the reaction, after the reaction solution is neutralized and filtered, the solvent is removed under reduced pressure.

The basic catalyst in the synthesis of the oxide-modified polyhydric alcohol is preferably alkali metal hydroxides and alkaline earth metal hydroxides and practically examples are sodium hydroxide and potassium hydroxide.

The solvent to be used in the synthesis of the oxide-modified polyhydric alcohol is not particularly limited if it is inactive against the reaction substances and preferable examples are aromatic hydrocarbons such as benzene, toluene, and xylene; aliphatic hydrocarbons such as n-hexane and n-heptane; and alicyclic hydrocarbons such as cyclohexane and cyclopentane.

The polyhydric alcohol is not particularly limited and examples are same as tri- or higher-hydric alcohol compounds described above.

The method for esterification reaction of the oxide-modified polyhydric alcohol and (meth)acrylic acid may be a common dehydration esterification reaction.

Practically, the method may involve loading the oxide-modified polyhydric alcohol, (meth)acrylic acid, and a solvent into a reactor equipped with a stirrer, a thermometer, and a water separator, heating the mixture in the presence of an acidic catalyst. Water produced as the reaction proceeds is removed to the outside of the system. The reaction solution is washed after completion of the reaction, and after the water-phase layer is separated, the solvent is removed under decreased pressure.

The solvent in the esterification reaction of the method (4) is not particularly limited if it makes water removal easy and is not reactive with (meth)acrylic acid, the oxide-modified polyhydric alcohol and an acidic catalyst, and preferable examples are the same solvent exemplified in the epoxylation reaction of the method (1).

The acidic catalyst in the esterification reaction of the method (4) may be the same acidic catalyst exemplified in the method (1). The addition amount is preferably 0.5% by weight in the lower limit and 5% by weight in the upper limit to the entire amount of the reaction solution.

The reaction temperature of the epoxylation reaction of the method (4) is preferably 70° C. in the lower limit and 150° C. in the upper limit. Heating at a temperature in the range makes it easy to carry out dehydration esterification reaction. It is more preferably 80° C. in the lower limit and 120° C. in the upper limit.

In the esterification reaction of the method (4), it is preferable to carry out reaction by adding a polymerization inhibitor. Examples of the polymerization inhibitor are the same polymerization inhibitor exemplified in the esterification reaction of the method (1) and the use amount is, in general, preferably 0.01% by weight in the lower limit and 1% by weight in the upper limit to the entire amount of the reaction solution.

The aimed (meth)acrylate may be obtained also by reaction of the oxide-modified polyhydric alcohol with acid halides such as (meth)acrylic acid chloride.

The carboxylic acid compound having two or more carboxyl groups, acid anhydride, and the method for addition reaction of the compound having two or more carboxyl groups and/or acid anhydride to the lactone-modified and/or oxide-modified compound having two or more polymerizable unsaturated bonds and a hydroxyl group in a molecule may be same respectively as those compounds and method described in the case of addition reaction of the compound having two or more carboxyl groups and/or acid anhydride to the compound having two or more polymerizable unsaturated bonds and a hydroxyl group in a molecule.

In the curable resin composition for a column spacer of the sixth present invention, the content of the polymerizable compound according to the present invention is not particularly limited, however, it is preferably 20% by weight in the lower limit and 90% by weight in the upper limit to the solid matter of the curable resin composition for a column spacer of the sixth present invention. If it is lower than 20% by weight, the curable resin composition for a column spacer of the sixth present invention is not sufficiently photo-cured and accordingly it may be sometimes impossible to form the pattern of the column spacer by photolithography in the case of the column spacer application. If it exceeds 90% by weight, the solubility in an alkaline developer solution becomes insufficient at the time of the column spacer production using the curable resin composition for a column spacer of the sixth present invention and accordingly the developability of the pattern of the column spacer to be produced mat sometimes become insufficient. It is more preferably 40% by weight in the lower limit and 80% by weight in the upper limit.

The curable resin composition for a column spacer of the six present invention may use a compound having a polymerizable unsaturated bond but no carboxyl group in a molecule (hereinafter, simply referred to as a polymerizable unsaturated bond-containing compound) in addition to the polymerizable compound according to the sixth present invention to adjust the reactivity and the developability to an extent that the flexibility and the developability of the column spacer to be produced are not deteriorated in the case of using the curable resin composition for the column spacer of the sixth present invention for the column spacer application.

Non-limiting examples of the polymerizable unsaturated bond-containing compound may be, as difunctional examples, neopentyl glycol di(meth)acrylate, 3-methyl-1,5-pentanediol di(meth)acrylate, 2-butyl-2-ethyl-1,3-propanediol di(meth)acrylate, 1,4-butanediol diacrylate, 1,6-hexanediol diacrylate, hydroxypivalic acid neopentyl glycol ester diacrylate; polyethylene glycol (meth)acrylates such as diethylene glycol (meth)acrylate, triethylene glycol (meth)acrylate, tetraethylene glycol (meth)acrylate, hexaethylene glycol (meth)acrylate, and nonaethylene glycol (meth)acrylate; and polyethylene glycol di(meth)acrylates such as diethylene glycol di(meth)acrylate, triethylene glycol di(meth)acrylate, tetraethylene glycol di(meth)acrylate, hexaethylene glycol di(meth)acrylate, and nonaethylene glycol di(meth)acrylate.

Further, the examples may include, as tri- or higher-functional examples, polyfunctional (meth)acrylate compounds such as trimethylolethane tri(meth)acrylate, trimethylolpropane tri(meth)acrylate, ditrimethylolpropane tetra(meth)acrylate, pentaerythritol tri(meth)acrylate, pentaerythritol tetra(meth)acrylate, dipentaerythritol tetra(meth)acrylate, dipentaerythritol penta(meth)acrylate, and dipentaerythritol hexa(meth)acrylate.

In the case the curable resin composition for a column spacer of the sixth present invention contains the polymerizable unsaturated bond-containing compound, the addition amount is not particularly limited, however, it is preferable to be lower than 40% by weight in total of the polymerizable compound according to the present invention and this compound. If it exceeds 40% by weight, the flexibility of the column spacer to be obtained is deteriorated and the effect of suppressing the gravity defect and cold bubble tends to be lowered. It is preferably 30% by weight in the upper limit.

The curable resin composition for a column spacer of the sixth present invention contains an alkali-soluble polymer compound.

Examples of the alkali-soluble polymer compound are the same alkali-soluble polymer compound exemplified in the curable resin composition for a column spacer of the first invention.

In the curable resin composition for a column spacer of the sixth present invention, the content of the alkali-soluble polymer compound is not particularly limited, however, it is preferably 10% by weight in the lower limit and 80% by weight in the upper limit. If it is lower than 10% by weight, the solubility in an alkaline developer solution becomes insufficient at the time of the column spacer production using the curable resin composition for a column spacer of the sixth present invention and accordingly the developability of the pattern of the column spacer to be produced may sometimes become insufficient, and if it exceeds 80% by weight, the curable resin composition for a column spacer of the second present invention is not sufficiently photo-cured and accordingly it may be sometimes impossible to form the pattern of the column spacer by photolithography. It is more preferably 20% by weight in the lower limit and 60% by weight in the upper limit.

The curable resin composition for a column spacer of the second present invention contains a photo-reaction initiator.

Examples of the photo-reaction initiator are the same photo-reaction initiator exemplified in the curable resin composition for a column spacer of the first present invention.

In the curable resin composition for a column spacer of the sixth present invention, the content of the photo-reaction initiator is not particularly limited, however, it is preferably 1% by weight in the lower limit and 20% by weight in the upper limit. If it is lower than 1% by weight, the curable resin composition for a column spacer of the sixth present invention is not sufficiently photo-cured, and if it exceeds 20% by weight, alkali development may be sometimes impossible in photolithography. It is more preferably 5% by weight in the lower limit and 15% by weight in the upper limit.

The curable resin composition for a column spacer of the first, second, third, fourth, fifth, or sixth present invention may contain a reaction aid for decreasing the reaction hindrance by oxygen. Use of such a reaction aid and a hydrogen abstraction type photo-reaction initiator in combination improves the curing speed when the light is radiated.

Examples of the reaction aid are amines such as n-butylamine, di-n-butylamine, triethylamine, triethylenetetramine, ethyl p-dimethylaminobenzoate, and isoamyl p-dimethylaminobenzoate; phosphines such as tri-n-butylphosphine; and sulfonic acids such as s-benzylisothiuronium-p-toluenesulfinate. These reaction aids may be used alone or two or more of them may be used in combination.

The curable resin composition for a column spacer of the first, second, third, fourth, fifth, or sixth present invention is preferable to contain further a compound having two or more block isocynato groups. The compound having two or more block isocynato groups works as a heat crosslinking agent and containing the compound having two or more blocking isocynato groups gives the thermosetting property to the curable resin composition for a column spacer of the first, second, third, fourth, fifth, or sixth present invention.

Non-limiting examples of the compound having two or more block isocynato groups are compounds obtained by blocking polyfunctional isocyanates comprising tolylene diisocyanate, 4,4-diphenylmethane diisocyanate, xylylene diisocyanate, hexamethylene diisocyanate, isophorone diisocyanate, methylenebis(4-cyclohexylisocyanate), trimethylhexamethylene diisocyanate, and their oligomers with active methylene type, oxime type, lactam type, or alcohol type blocking agent compounds. These compounds having two or more block isocynato groups may be used alone or two or more of them may be used in combination.

Commercialized products as the compound having two or more block isocynato groups are Duranate 17B-60PX and Duranate E-402-B80T (manufactured by Asahi Kasei Chemicals Corporation).

In the case the curable resin composition for a column spacer of the first, second, third, fourth, fifth, or sixth present invention contains the compound having two or more block isocynato groups, the content of the compound is preferably 0.01 parts by weight in the lower limit and 50 parts by weight in the upper limit to 100 parts by weight of the alkali-soluble polymer compound. If it is lower than 0.01 parts by weight, the curable resin composition for a column spacer of the first, second, third, fourth, fifth, or sixth present invention cannot be sometimes sufficiently thermal-cured, and if it exceeds 50 parts by weight, the crosslinking degree of the obtained cured product may sometimes become too high to satisfy the elastic property as described later. It is more preferably 0.05 parts by weight in the lower limit and 20 parts by weight in the upper limit.

The curable resin composition for a column spacer of the first, second, third, fourth, fifth, or sixth present invention may be diluted with a diluent to adjust the viscosity.

The diluent is not particularly limited and may be selected properly in consideration of the compatibility with the curable resin composition for a column spacer of the first, second, third, fourth, fifth, or sixth present invention, coating method, film evenness at the time of drying, drying efficiency and the like, however, in the case the curable resin composition for a column spacer of the first, second, third, fourth, fifth, or sixth present invention is coated by using a spin coater or a slit coater, preferable examples are organic solvents such as methyl cellosolve, ethyl cellosolve, ethyl cellosolve acetate, diethylene glycol dimethyl ether, propylene glycol monoethyl ether acetate, and isopropyl alcohol. These diluents may be used alone or two or more of them may be used in combination.

If necessary, the curable resin composition for a column spacer of the first, second, third, fourth, fifth, or sixth present invention may contain conventionally known additives such as a silane coupling agent for improving the adhesion to a substrate.

Use of the curable resin composition for a column spacer of the first, second, third, fourth, fifth, or sixth present invention makes it possible to produce a column spacer satisfying both high recovery property from the compressive deformation and flexibility and low elastic modulus by photo-curing (and thermal-curing) and also to obtain sharp resolution without leaving development residues at the time of pattern formation. Further, use of such a column spacer makes it possible to obtain a liquid crystal display panel with efficiently suppressed occurrence of color irregularity due to gravity defect without generating cold bubble.

The elastic coefficient of a cured product at 25° C. and 15% contraction which is obtained by curing the curable resin composition for a column spacer of the first, second, third, fourth, fifth, or sixth present invention by light radiation (and heating) is preferably 0.2 GPa in the lower and 1.0 GPa in the upper limit. If it is lower than 0.2 GPa, the cured product is so soft that it is difficult to keep the cell gap, and if it exceeds 1.0 GPa, the cured product becomes so hard to penetrate a color filter layer at the time of sticking substrates or fails to obtain sufficient elastic deformation necessary for recovery. It is more preferably 0.3 GPa in the lower and 0.9 GPa in the upper limit and even more preferably 0.5 GPa in the lower and 0.7 GPa in the upper limit.

In this description, the cured product means a cured product obtained by almost completely curing the curable resin composition for a column spacer of the first, second, third, fourth, fifth, or sixth present invention. The condition for almost complete curing is radiating ultraviolet rays with at least 50 mJ/cm² intensity and further in the case of heating, heating treatment at a temperature of 200 to 250° C. for about 20 minutes.

In this description, 15% compression means compression caused in a manner that the deformation ratio of the height of the column spacer becomes 15%. Further, the elastic coefficient and recovery ratio are measured by the following methods.

That is, at first, a column spacer formed on a substrate is compressed at a load application speed of 10 mN/s until the height is equivalent to 85% of the initial height H₀. In this case, H₁ is defined as the column spacer height when 1 mN load is applied, and H₂ is defined as the column spacer height corresponding to 85% of H₀, and the load at the time when column spacer height reaches H₂ is defined as F. Next, after the load F is kept for 5 seconds to cause deformation at the constant load, the load is removed at 10 mN/s load application speed to measure the recovery deformation of the height of the column spacer due to the elastic recovery. The column spacer height at the time when the compressive deformation becomes the maximum is defined as H₃, and the column spacer height at the time when 1 mN load is applied during the recovery from the deformation of the column spacer is defined as H₄. The elastic coefficient and recovery ratio are calculated according to the following equations (1) and (2).

Elastic coefficient E=F/(D×S) (1)

Recovery ratio R=(H₄−H₃)/(H₁−H₃)×100 (2)

In the formula (1), F denotes the load (N); D denotes the deformation ratio of height of a column spacer; and S denotes the cross-section surface area (m²) of a column spacer.

A method for producing the curable resin composition for a column spacer of the first, second, third, fourth, fifth, or sixth present invention is not particularly limited and an example may be a method of mixing the compound having two or more polymerizable unsaturated bonds, an alkali-soluble polymer compound, a photo-reaction initiator, and if necessary, a polymerizable unsaturated bond-containing compound, a compound having two or more block isocyanate groups, and a diluent by a conventionally known mixing method.

Next, a method for producing a column spacer using the curable resin composition for a column spacer of the first, second, third, fourth, fifth, or sixth present invention will be described.

In the case of producing a column spacer using the curable resin composition for a column spacer of the first, second, third, fourth, fifth, or sixth present invention, at first a coating is formed by applying the curable resin composition for a column spacer of the first, second, third, fourth, fifth, or sixth present invention to a substrate in a predetermined thickness.

Non-limiting examples of the coating method may be conventional coating methods such as spin coat, slit coat, spray coat, dip coat, and bar coat.

Next, active light ray such as ultraviolet ray is radiated through a mask having predetermined patterns to the formed coating. Accordingly, in the light radiation parts, the compound having two or more polymerizable unsaturated bonds in a molecule and photo-reaction initiator contained in the curable resin composition for a column spacer of the first, second, third, fourth, fifth, or sixth present invention are reacted each other to photo-cure the composition.

The radiation dose of the active light ray is not particularly limited, however, in the case of ultraviolet ray, it is preferably 100 mJ/cm² or higher. If it is lower than 100 mJ/cm², the photo-curing may be sometime insufficient so that even the exposed parts are dissolved by the alkali treatment to be carried out successively and accordingly patterns cannot be formed.

Next, the photo-cured product after the photo-curing are alkali developed to form a column spacer of the predetermined pattern, which comprises the photo-cured product of the curable resin composition for a column spacer of the first, second, third, fourth, fifth, or sixth present invention.

Since the curable resin composition for a column spacer of the first, second, third, fourth, fifth, or sixth present invention contains the compound having two or more polymerizable unsaturated bonds with above-mentioned specified structure in a molecule, it is possible to form a column spacer in sharp patterns with excellent resolution while residues are scarcely generated at the time of predetermined pattern formation in this process. Further, the column spacer produced by using the curable resin composition for a column spacer of the third, fourth, or fifth present invention have higher recovery property from the compressive deformation and flexibility and low elastic modulus.

In the case the curable resin composition for a column spacer of the first, second, third, fourth, fifth, or sixth present invention contains a compound having two or more block isocyanate groups, if the patterned photo-cured product after the development treatment is heated, reaction of the alkali-soluble polymer compound contained in the composition and the compound having two or more block isocyanate groups is caused.

The heating condition may be determined properly in consideration of the size and thickness of the patterns, however, it is preferably 200° C. for 20 minutes at least.

The present invention also include a column spacer produced by using the curable resin composition for a column spacer of the first, second, third, fourth, fifth, or sixth present invention.

The column spacer of the present invention is preferable to have an elastic coefficient of 0.2 GPa in the lower limit and 1.0 GPa in the upper limit at the time of 15% compression at 25° C. If it is lower than 0.2 GPa, the column spacer becomes so soft that it is difficult to keep the cell gap and if it exceeds 1.0 GPa, the column spacer becomes so hard to penetrate a color filter layer at the time of sticking substrates or fail to obtain sufficient elastic deformation necessary for recovery. It is more preferably 0.3 GPa in the lower limit and 0.9 GPa in the upper limit and even more preferably 0.5 GPa in the lower limit and 0.7 GPa in the upper limit.

The column spacer of the present invention is produced by a conventional method such as ODF method while the height is planed to be slightly higher than the cell gap, so that a liquid crystal display panel which can efficiently suppress occurrence of color irregularity due to gravity defect without causing cold bubble can be produced.

The present invention also include a liquid crystal display panel produced by using the curable resin composition for a column spacer of the first, second, third, fourth, fifth, or sixth present invention or the column spacer of the present invention.

### EFFECTS OF THE INVENTION

According to the present invention, it is made possible to provide a curable resin composition for a column spacer which has excellent developability and solubility and is capable of forming a clearly patterned column spacer without leaving a development residue at the time of pattern formation of the column spacer to be used in producing a liquid crystal display panel; a curable resin composition for a column spacer which is capable of forming a clearly patterned column spacer without leaving a development residue at the time of pattern formation of the column spacer to be used in producing a liquid crystal display panel and capable of obtaining a liquid crystal display panel capable of effectively suppressing occurrence of color irregularity due to gravity defect without generating cold bubble; a column spacer obtained by using the curable resin composition for a column spacer; and a liquid crystal display panel.

### BEST MODE FOR CARRYING OUT THE INVENTION

Hereinafter, the present invention will be described in details with reference to examples, however the present invention is not limited to these examples.

### Example 1

### (1) Synthesis of Alkali-Soluble Polymer Compound

After 60 parts by weight of diethylene glycol dimethyl ether as a solvent was added to a 3 L-capacity separable flask and heated to 90° C. under nitrogen atmosphere, 10 parts by weight of methyl methacrylate, 8 parts by weight of methacrylic acid, 12 parts by weight of n-butyl methacrylate, 10 parts by weight of 2-ethylhexyl acrylate, 0.4 parts by weight of azobisvaleronitrile, and 0.8 parts by weight of n-dodecylmercaptan were continuously dropped for 3 hours.

After that, held at 90° C. for 30 minutes, the mixture was heated to 105° C. and polymerization was continued for 3 hours to obtain an alkali-soluble polymer compound solution.

The obtained alkali-soluble polymer compound was sampled and subjected to molecular weight measurement by gel permeation chromatography (GPC) to find that the weight average molecular weight (Mw) was about 20000.

### (2) Preparation of a Curable Resin Composition for a Column Spacer

A curable resin composition for a column spacer was prepared by mixing 100 parts by weight (solid matter ratio 40 wt %) of the obtained alkali-soluble polymer compound solution, 60 parts by weight of ethylene oxide-modified pentaerythritol tetraacrylate (a compound obtained by reaction of 1 mole of pentaerythritol and 35 mole of ethylene oxide and then esterification of 1 mole of the reaction product with 4 mole of acrylic acid; manufactured by Shin-Nakamura Chemical Co., Ltd.), 10 parts by weight of Irgacure 907 (manufactured by Ciba Specialty Chemicals Inc.) and 10 parts by weight of DETX-S (manufactured by Nippon Kayaku Co., Ltd.) as a photo-reaction initiator, and 70 parts by weight of diethylene glycol dimethyl ether as a solvent.

### Example 2

A curable resin composition for a column spacer was prepared by mixing 100 parts by weight of the alkali-soluble polymer compound solution obtained in Example 1, 80 parts by weight of propylene oxide-modified trimethylolpropane triacrylate (a compound obtained by reaction of 1 mole of trimethylolpropane and 20 mole of propylene oxide and then esterification of 1 mole of the reaction product with 3 mole of acrylic acid; manufactured by Shin-Nakamura Chemical Co., Ltd.), 10 parts by weight of Irgacure 907 (manufactured by Ciba Specialty Chemicals Inc.) and 10 parts by weight of DETX-S (manufactured by Nippon Kayaku Co., Ltd.) as a photo-reaction initiator, and 70 parts by weight of diethylene glycol dimethyl ether as a solvent.

### Example 3

A curable resin composition for a column spacer was prepared by mixing 100 parts by weight (solid matter ratio 40 wt %) of Cyclomer P ACA-230AA (manufactured by Daicel Chem. Ind., Ltd.) as an alkali-soluble polymer compound, 60 parts by weight of ethylene oxide-modified pentaerythritol triacrylate (a compound obtained by reaction of 1 mole of pentaerythritol and 30 mole of ethylene oxide and then esterification of 1 mole of the reaction product with 3 mole of acrylic acid), 10 parts by weight of Irgacure 907 (manufactured by Ciba Specialty Chemicals Inc.) and 10 parts by weight of DETX-S (manufactured by Nippon Kayaku Co., Ltd.) as a photo-reaction initiator, and 70 parts by weight of diethylene glycol dimethyl ether as a solvent.

### Example 4

A curable resin composition for a column spacer was prepared by mixing 100 parts by weight (solid matter ratio 40 wt %) of the alkali-soluble polymer compound solution obtained in Example 1, 60 parts by weight of ethylene oxide/caprolactone-modified dipentaerythritol hexaacrylate (a compound obtained by reaction of 1 mole of dipentaerythritol and 12 mole of ethylene oxide and then esterification of 1 mole of the reaction product with 6 mole of a reaction product obtained by reaction of 1 mole of acrylic acid and 2 mole of caprolactone), 10 parts by weight of Irgacure 907 (manufactured by Ciba Specialty Chemicals Inc.) and 10 parts by weight of DETX-S (manufactured by Nippon Kayaku Co., Ltd.) as a photo-reaction initiator, and 70 parts by weight of diethylene glycol dimethyl ether as a solvent.

### Example 5

A curable resin composition for a column spacer was prepared by mixing 100 parts by weight of the alkali-soluble polymer compound solution obtained in Example 1, 120 parts by weight of propylene oxide/caprolactone-modified pentaerythritol triacrylate (a compound obtained by reaction of 1 mole of dipentaerythritol and 6 mole of propylene oxide and then esterification of 1 mole of the reaction product with 6 mole of a reaction product obtained by reaction of 1 mole of acrylic acid and 2 mole of caprolactone), 15 parts by weight of photo-reaction initiator (Irgacure 369, manufactured by Ciba Specialty Chemicals Inc.), 8 parts by weight of heat crosslinking agent (Duranate E-402-B80T, manufactured by Asahi Kasei Chemicals Corporation), and 60 parts by weight of diethylene glycol dimethyl ether as a solvent.

### Example 6

A curable resin composition for a column spacer was prepared by mixing 100 parts by weight (solid matter ratio 40 wt %) of Cyclomer P ACA-230AA (manufactured by Daicel Chem. Ind., Ltd.) as an alkali-soluble polymer compound, 80 parts by weight of ethylene oxide/caprolactone-modified pentaerythritol tetraacrylate (a compound obtained by reaction of 1 mole of pentaerythritol and 8 mole of ethylene oxide and then esterification of 1 mole of the reaction product with 4 mole of a reaction product obtained by reaction of 1 mole of acrylic acid and 2 mole of caprolactone), 15 parts by weight of a photo-reaction initiator (Irgacure 369, manufactured by Ciba Specialty Chemicals Inc.) as a photo-reaction initiator, and 60 parts by weight of diethylene glycol dimethyl ether as a solvent.

### Example 7

A curable resin composition for a column spacer was prepared by mixing 100 parts by weight (solid matter ratio 40 wt %) of Cyclomer P ACA-230AA (manufactured by Daicel Chem. Ind., Ltd.) and 100 parts by weight (solid matter ratio 40 wt %) of the alkali-soluble polymer compound solution obtained in Example 1 as an alkali-soluble polymer compound, 80 parts by weight of caprolactone-modified pentaerythritol triacrylate (a compound obtained by esterification of 1 mole of pentaerythritol and 3 mole of a reaction product obtained by reaction of 1 mole of acrylic acid and 2 mole of caprolactone), 10 parts by weight of Irgacure 907 (manufactured by Ciba Specialty Chemicals Inc.) and 10 parts by weight of DETX-S (manufactured by Nippon Kayaku Co., Ltd.) as a photo-reaction initiator, and 70 parts by weight of diethylene glycol dimethyl ether as a solvent.

### Example 8

A curable resin composition for a column spacer was prepared by mixing 100 parts by weight (solid matter ratio 40 wt %) of Cyclomer P ACA-230AA (manufactured by Daicel Chem. Ind., Ltd.) as an alkali-soluble polymer compound, 120 parts by weight of caprolactone-modified dipentaerythritol pentaacrylate (a compound obtained by esterification of 1 mole of dipentaerythritol and 5 mole of a reaction product obtained by reaction of 1 mole of acrylic acid and 2 mole of caprolactone), 15 parts by weight of a photo-reaction initiator (Irgacure 369, manufactured by Ciba Specialty Chemicals Inc.), and 60 parts by weight of diethylene glycol dimethyl ether as a solvent.

### Example 9

A curable resin composition for e column spacer was prepared by mixing 100 parts by weight (solid matter ratio 40 wt %) of Cyclomer P ACA-230AA (manufactured by Daicel Chem. Ind., Ltd.) and 100 parts by weight (solid matter ratio 40 wt %) of the alkali-soluble polymer compound solution obtained in Example 1 as an alkali-soluble polymer compound, 80 parts by weight of ethylene oxide/caprolactone-modified dipentaerythritol pentaacrylate (a compound obtained by reaction of 1 mole of dipentaerythritol and 12 mole of ethylene oxide and then esterification of 1 mole of the reaction product with 5 mole of a reaction product obtained by reaction of 1 mole of acrylic acid and 2 mole of caprolactone), 10 parts by weight of Irgacure 907 (manufactured by Ciba Specialty Chemicals Inc.) and 10 parts by weight of DETX-S (manufactured by Nippon Kayaku Co., Ltd.) as a photo-reaction initiator, and 70 parts by weight of diethylene glycol dimethyl ether as a solvent.

### Example 10

A monomer solution was prepared by adding 50 parts by weight (26 mmol) of caprolactone-modified dipentaerythritol hexaacrylate (manufactured by Nippon Kayaku Co., Ltd.) having a structure shown in the following chemical formula (2) as a raw material monomer, 0.025 parts by weight of hydroquinone as a polymerization inhibitor, and 40 parts by weight of methanol as a solvent to a flask and heating and mixing the mixture at 40° C.

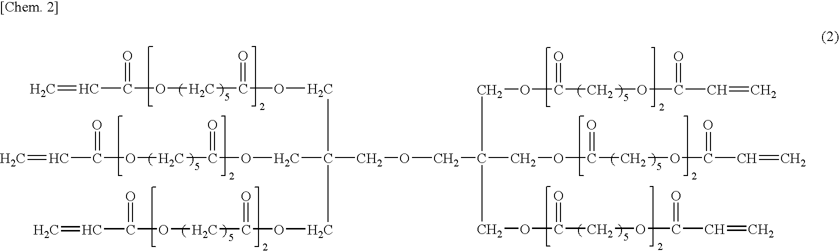

Next, a mixture containing 2.70 parts by weight (26 mmol) of diethanol amine and 10 parts by weight of methanol was dropped to the prepared monomer solution for 15 minutes and the reaction was carried out at 40° C. for 3 hours and the reaction mixture was cooled to a room temperature.

In a water bath at 50° C., the reaction mixture was treated by an evaporator for 30 minutes to 1 hour to obtain a compound (A) having two or more polymerizable unsaturated bonds in a molecule shown in the following chemical formula (3). The result of NMR measurement of the obtained compound (A) is shown in FIG. 1. Further, the caprolactone-modified dipentaerythritol hexaacrylate shown in the chemical formula (2) as a raw material monomer was also subjected to NMR measurement. The result is also shown in FIG. 5.

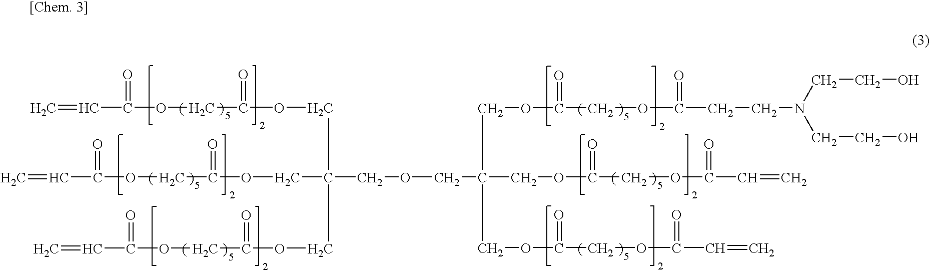

After that, a curable resin composition for a column spacer was prepared in the same manner as Example 1, except that the obtained compound (A) was used in place of the ethylene oxide-modified pentaerythritol tetraacrylate.

### Example 11

A compound (B) having two or more polymerizable unsaturated bonds in a molecule shown in the following chemical formula (4) was obtained in the same manner as Example 10, except that the addition amount of diethanol amine was changed to 5.40 parts by weight (51 mmol). The result of NMR measurement of the obtained compound (B) is shown in FIG. 2.

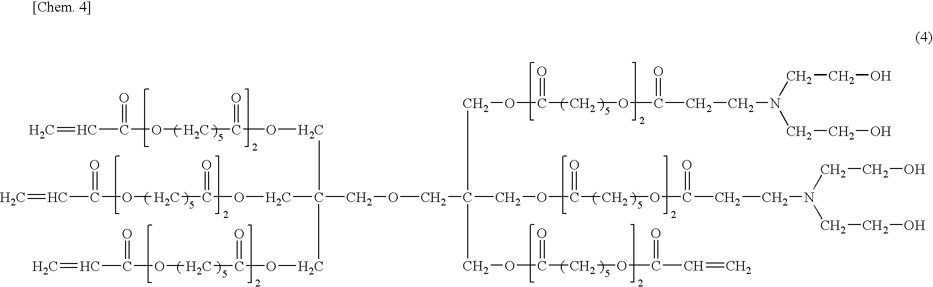

After that, a curable resin composition for a column spacer was prepared in the same manner as Example 1, except that the obtained compound (B) was used in place of the ethylene oxide-modified pentaerythritol tetraacrylate.

### Example 12

A compound (C) having two or more polymerizable unsaturated bonds in a molecule shown in the following chemical formula (5) was obtained in the same manner as Example 10, except that the addition amount of diethanol amine was changed to 8.09 parts by weight (77 mmol). The result of NMR measurement of the obtained compound (C) is shown in FIG. 3.

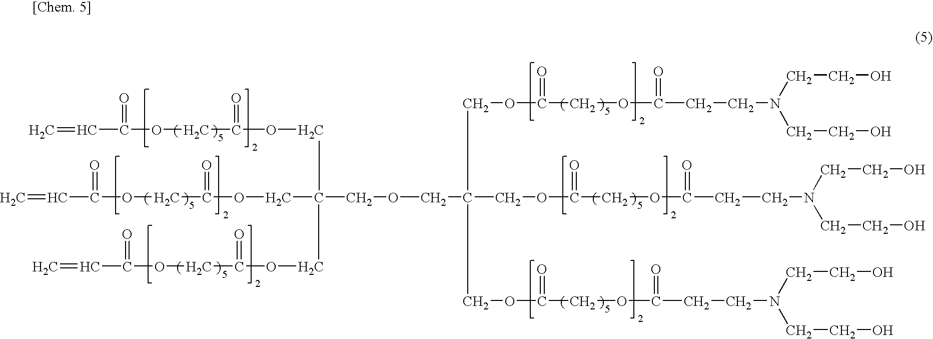

After that, a curable resin composition for a column spacer was prepared in the same manner as Example 1, except that the obtained compound (C) was used in place of the ethylene oxide-modified pentaerythritol tetraacrylate.

### Example 13

A monomer solution was prepared by adding 50 parts by weight (41 mmol) of caprolactone-modified pentaerythritol triacrylate shown in the following chemical formula (6) as a raw material monomer (a compound obtained by reaction of 8 mole of caprolactone and 3 mole of acrylic acid with 1 mole of pentaerythritol, manufactured by Shin-Nakamura Chemical Co., Ltd.), 0.025 parts by weight of hydroquinone as a polymerization inhibitor, and 40 parts by weight of methanol as a solvent to a flask and heating and mixing the mixture at 40° C.

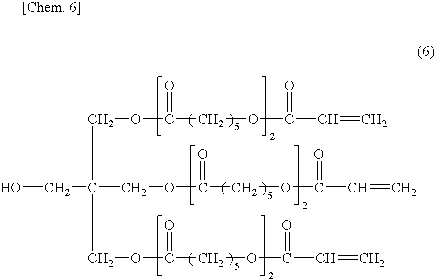

Next, a mixture containing 4.34 parts by weight (41 mmol) of diethanol amine and 10 parts by weight of methanol was dropped to the prepared monomer solution for 15 minutes and the reaction was carried out at 40° C. for 3 hours and the reaction mixture was cooled to a room temperature.

In a water bath at 50° C., the reaction mixture was treated by an evaporator for 30 minutes to 1 hour to obtain a compound (D) having two or more polymerizable unsaturated bonds in a molecule shown in the following chemical formula (7). The result of NMR measurement of the obtained compound (D) is shown in FIG. 4. Further, the caprolactone-modified pentaerythritol triacrylate was also subjected to NMR measurement. The result is also shown in FIG. 6.

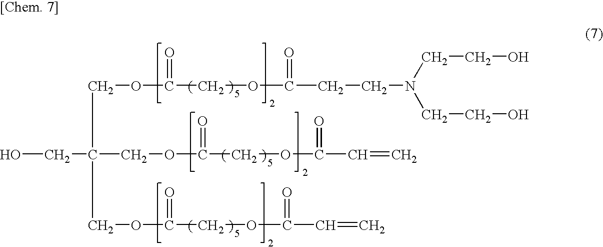

After that, a curable resin composition for a column spacer was prepared in the same manner as Example 1, except that the obtained compound (D) was used in place of the ethylene oxide-modified pentaerythritol tetraacrylate.

### Comparative Example 1

A curable resin composition for a column spacer was prepared by mixing 100 parts by weight (solid matter ratio 40 wt %) of the alkali-soluble polymer compound solution obtained in Example 1, 80 parts by weight of caprolactone-modified dipentaerythritol hexaacrylate (DPCA-120, manufactured by Nippon Kayaku Co., Ltd.), 10 parts by weight of Irgacure 907 (manufactured by Ciba Specialty Chemicals Inc.) and 10 parts by weight of DETX-S (manufactured by Nippon Kayaku Co., Ltd.) as a photo-reaction initiator, and 70 parts by weight of diethylene glycol dimethyl ether as a solvent.

### Comparative Example 2

A curable resin composition for a column spacer was prepared by mixing 100 parts by weight (solid matter ratio 40 wt %) of the alkali-soluble polymer compound solution obtained in Example 1, 80 parts by weight of dipentaerythritol hexaacrylate (DPHA, manufactured by Nippon Kayaku Co., Ltd.), 10 part by weight of Irgacure 907 (manufactured by Ciba Specialty Chemicals Inc.) and 10 parts by weight of DETX-S (manufactured by Nippon Kayaku Co., Ltd.) as a photo-reaction initiator, and 70 parts by weight of diethylene glycol dimethyl ether as a solvent.

### Comparative Example 3

A curable resin composition for a column spacer was prepared by mixing 100 parts by weight (solid matter ratio 40 wt %) of “Cyclomer P ACA-200” (manufactured by Daicel Chem. Ind., Ltd.), 13.7 parts by weight of pentaerythritol triacrylate (PET-30, manufactured by Kyoeisha Chemical Co., Ltd.), 3.4 parts by weight of caprolactone-modified dipentaerythritol hexaacrylate (DPCA-60, manufactured by Nippon Kayaku Co., Ltd.), 15 parts by weight of photo-reaction initiator (Irgacure 369, manufactured by Ciba Specialty Chemicals Inc.), and 60 parts by weight of diethylene glycol dimethyl ether as a solvent.

### (Evaluation)

The curable resin compositions for a column spacer obtained in Examples 1 to 13 and Comparative Examples 1 to 3 were evaluated by the following methods. The respective results are shown in Table 1.

### (1) Production of a Column Spacer

Each of the curable resin compositions obtained in respective Examples and Comparative Examples was coated by spin coating to a glass substrate in which a transparent conductive coating was formed and dried at 100° C. for 2 minutes to form a coating film. The obtained coating film was subjected to ultraviolet radiation of 100 mJ/cm² intensity through a 20 μm-square dotted-pattern mask and successively developed with a 0.04% KOH solution and washed with pure water for 30 seconds to obtain a column spacer pattern.

After that, when baking treatment was carried out at 220° C. for 30 minutes, the cross-section surface area of each column spacer was 20 μm×20 μm (400 μm²) and the height was 3.0 μm.

### (Evaluation of a Column Spacer)

### (Resolution)

The sharpness (resolution) of the edge of each column spacer pattern and roughness of the pattern surface (pattern formation state) were observed by an optical microscope and evaluated according to the following evaluation standards.

Evaluation of resolution
◯: sharp state
X: uneven state

Pattern formation state

◯: even state

X: roughened state

### (Alkali-Solubility)

The alkali-solubility was evaluated according to the following standard by dissolving 1 part by weight of the polymerizable compound (solid matter) in 200 parts by weight of an aqueous 0.04 wt % KOH solution and observing the dissolution state with eyes.

⊚: completely dissolved without turbidity or precipitate in the solution
◯: becoming turbid, but no precipitate in the solution
Δ: becoming turbid and precipitate observed in the solution
X: precipitated without being dissolved

### (Compression Property)

In a chamber adjusted at 25° C., each column spacer was compressed at load-applied speed of 10 mN/s until the height was compressed to the height corresponding to 85% of the initial height H₀. In this case, H₁ was defined as the column spacer height when 1 mN load was applied: H₂ was defined as the column spacer height corresponding to 85% of H₀: and F was defined as the load at the time when the column spacer height reached H₂.

Next, after the load F was kept for 5 seconds to cause deformation at the constant load, the load was relieved at 10 mN/s load-applied speed to measure the recovery deformation of the height of the column spacer due to the elastic recovery. The column spacer height at the time when the compressive deformation becomes the maximum was defined as H₃ and the column spacer height at the time when 1 mN load was applied during the recovery from the deformation of the column spacer was defined as H₄. By using the obtained each value, the compressive elastic coefficient E at the 15% compression and recovery ratio R at the 15% compression deformation were calculated according to the following equations (1) and (2). In the formula (1), E denotes the compressive elastic coefficient (Pa); F denotes load (N); D denotes the deformation ratio ((H₀-H₂)/H₀) of height of a column spacer; and S denotes the cross-section surface area (m²) of a column spacer.

E=F/(D×S) (1)

R=(H₄−H₃)/(H₁−H₃)×100 (2)

### (2) Production of a Liquid Crystal Dispaly Panel

A seal agent (manufactured by Sekisui Chem. Co., Ltd.) was coated to the glass substrate on which the obtained column spacer was formed in a manner that a rectangular frame was drawn.

Successively micro droplets of a liquid crystal (JC-5004LA, manufactured by Chisso Corp.) were dropped and coated to the entire surface in the frame of the glass substrate and immediately the other glass substrate was laminated and ultraviolet ray was radiated to the seal part at 50 mW/cm² intensity for 60 seconds using a high pressure mercury lamp.

After that, liquid crystal annealing was carried out at 120° C. for 1 hour for heat-curing to produce a liquid crystal display panel.

### (Evaluation of a Liquid Crystal Display Panel)

Each liquid crystal display panel was illuminated and displayed, and the evenness of the cell gap was observed by observing the display screen with eyes and evaluated according to the following standard.

Further, each liquid crystal display panel was left for 60 hours at 60° C. while being perpendicularly stood. After that, the liquid crystal display panel was set between Cross Nicols and the display screen was observed with eyes to evaluate occurrence of gravity defect according to the following standard.

Further, each liquid crystal display panel was left for 24 hours at −20° C. After that, the liquid crystal display panel was set between Cross Nicols and observed with eyes to evaluate occurrence of cold bubble according to the following standard.

Evaluation of cell gap
◯: even
X: color irregularity occurred
Evaluation of gravity defect
◯: even
X: color irregularity occurred
Evaluation of cold bubble
◯: no foaming occurred
X: foaming occurred

### Example 14

### (1) Synthesis of a Compound Having Polymerizable Unsaturated Bonds

A 1 L-capacity eggplant-shaped flask was loaded with 100 parts by weight of methanol as a solvent, 40 parts by weight of caprolactone-modified dipentaerythritol hexaacrylate (DPCA-120, manufactured by Nippon Kayaku Co., Ltd.), 4 parts by weight of thiosalicylic acid (manufactured by Wako Pure Chemical Industries, Ltd.) as a polyfunctional (meth)acrylate compound, 0.05 parts by weight of an aqueous solution containing 40% of benzyltrimethylammonium hydroxide (manufactured by Wako Pure Chemical Industries, Ltd.) as a catalyst, and 0.4 parts by weight of hydroquinone as a polymerization inhibitor and under stirring condition, reaction was carried out at a room temperature for 1 hour.

After that, methanol was removed by an evaporator and caprolactone-modified dipentaerythritol hexaacrylate having carboxyl group was obtained.

### (2) Synthesis of an Alkali-Soluble Polymer Compound

After 60 parts by weight of diethylene glycol dimethyl ether as a solvent was added to a 3 L-capacity separable flask and heated to 90° C. under nitrogen atmosphere, 10 parts by weight of methyl methacrylate, 8 parts by weight of methacrylic acid, 12 parts by weight of n-butyl methacrylate, 10 parts by weight of 2-ethylhexyl acrylate, 0.4 parts by weight of azobisvaleronitrile, and 0.8 parts by weight of n-dodecylmercaptan were continuously dropped for 3 hours.

After that, held at 90° C. for 30 minutes, the mixture was heated to 105° C. and polymerization was continued for 3 hours to obtain an alkali-soluble polymer compound solution.

The obtained alkali-soluble polymer compound was sampled and subjected to molecular weight measurement by gel permeation chromatography (GPC) to find that the weight average molecular weight (Mw) was about 20000.

### (3) Preparation of a Curable Resin Composition

A curable resin composition was prepared by mixing 100 parts by weight (solid matter ratio 40 wt %) of the obtained alkali-soluble polymer compound solution, 60 parts by weight of carboxyl-containing caprolactone-modified dipentaerythritol hexaacrylate, 10 parts by weight of Irgacure 907 (manufactured by Ciba Specialty Chemicals Inc.) and 10 parts by weight of DETX-S (manufactured by Nippon Kayaku Co., Ltd.) as a photo-reaction initiator, and 70 parts by weight of diethylene glycol dimethyl ether as a solvent.

### Example 15

### (1) Synthesis of a Compound Having Polymerizable Unsaturated Bonds

A 1 L-capacity eggplant-shaped flask was loaded with 100 parts by weight of methanol as a solvent, 40 parts by weight of caprolactone-modified pentaerythritol tetraacrylate (a compound obtained by reaction of 1 mole of acrylic acid and 2 mole of caprolactone and then esterification of 4 mole of the reaction product with 1 mole of pentaerythritol), 4 parts by weight of mercaptopropionic acid (manufactured by Wako Pure Chemical Industries, Ltd.) as a polyfunctional (meth)acrylate compound, 0.05 parts by weight of an aqueous solution containing 40% of benzyltrimethylammonium hydroxide (manufactured by Wako Pure Chemical Industries, Ltd.) as a catalyst, and 0.4 parts by weight of hydroquinone as a polymerization inhibitor, and reaction was carried out at a room temperature for 1 hour under stirring condition.

After that, methanol was removed by an evaporator, and caprolactone-modified pentaerythritol tetraacrylate containing carboxyl group was obtained.

### (2) Preparation of a Curable Resin Composition

A curable resin composition was prepared by mixing 100 parts by weight (solid matter ratio 40 wt %) of Cyclomer P ACA-230AA (manufactured by Daicel Chem. Ind., Ltd.) as an alkali-soluble polymer compound, 60 parts by weight of caprolactone-modified pentaerythritol tetraacrylate containing carboxyl group obtained in (1) as a polymerizable unsaturated bond-containing compound, 10 parts by weight of Irgacure 907 (manufactured by Ciba Specialty Chemicals Inc.) and 10 parts by weight of DETX-S (manufactured by Nippon Kayaku Co., Ltd.) as a photo-reaction initiator, and 70 parts by weight of diethylene glycol dimethyl ether as a solvent.

### Example 16

A curable resin composition was prepared by mixing 100 parts by weight of the alkali-soluble polymer compound solution obtained in Example 14, 120 parts by weight of carboxyl-containing caprolactone-modified pentaerythritol triacrylate, 15 parts by weight of photo-reaction initiator (Irgacure 369, manufactured by Ciba Specialty Chemicals Inc.), 8 parts by weight of a heat crosslinking agent (Duranate E-402-B80T, manufactured by Asahi Kasei Chemicals Corporation), and 60 parts by weight of diethylene glycol dimethyl ether as a solvent.

### Example 17

### (1) Synthesis of a Compound Having Polymerizable Unsaturated Bonds

A 1 L-capacity eggplant-shaped flask was loaded with 100 parts by weight of methanol as a solvent, 40 parts by weight of ethylene oxide/caprolactone-modified dipentaerythritol tetraacrylate (a compound obtained by reaction of 1 mole of acrylic acid and 2 mole of caprolactone and then esterification of 6 mole of the reaction product with 1 mole of a reaction product obtained by reaction of 1 mole of dipentaerythritol and 12 mole of ethylene oxide), 4 parts by weight of mercaptosuccinic acid (manufactured by Wako Pure Chemical Industries, Ltd.) as a polyfunctional (meth)acrylate compound, 0.05 parts by weight of an aqueous solution containing 40% of benzyltrimethylammonium hydroxide (manufactured by Wako Pure Chemical Industries, Ltd.) as a catalyst, and 0.4 parts by weight of hydroquinone as a polymerization inhibitor, and reaction was carried out at 50° C. for 1 hour under stirring condition.

After that, methanol was removed by an evaporator and carboxyl-containing ethylene oxide/caprolactone-modified dipentaerythritol tetraacrylate was obtained.

### (2) Preparation of a Curable Resin Composition

A curable resin composition was prepared by mixing 100 parts by weight of the alkali-soluble polymer compound solution obtained in Example 14, 60 parts by weight of carboxyl-containing ethylene oxide/caprolactone-modified dipentaerythritol tetraacrylate obtained in (1) as a polymerizable unsaturated bond-containing compound, 10 parts by weight of Irgacure 907 (manufactured by Ciba Specialty Chemicals Inc.) and 10 parts by weight of DETX-S (manufactured by Nippon Kayaku Co., Ltd.) as a photo-reaction initiator, and 70 parts by weight of diethylene glycol dimethyl ether as a solvent.

### Example 18

A flask was loaded with 20 parts by weight (9.8 mmol) of a compound obtained by adding diethanolamine to caprolactone-modified dipentaerythritol hexaacrylate shown in the following chemical formula (8) as a raw material monomer, 1.48 parts by weight (9.8 mmol) of tetrahydrophthalic anhydride as an acid anhydride, 0.01 parts by weight of hydroquinone as a polymerization inhibitor, and 20 parts by weight of propylene glycol methyl ether acetate (PGMEA) as a solvent and the mixture was heated under nitrogen flow.

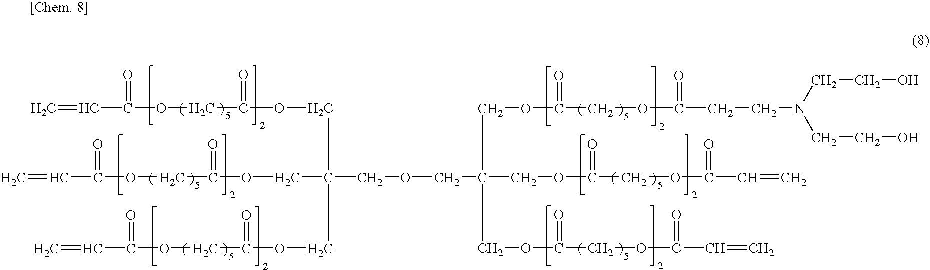

Next, when tetrahydrophthalic anhydride was completely dissolved, 0.02 parts by weight of triethylamine as a catalyst was added and after reaction was carried out in an oil bath at 120° C. for 6 hours under nitrogen atmosphere, the reaction product was cooled to a room temperature to obtain a compound (E) having a structure shown in the following chemical formula (9). The result of NMR measurement of the obtained compound (E) is shown in FIG. 7. Further, tetrahydrophthalic anhydride was also subjected to NMR measurement. The result is also shown in FIG. 11.

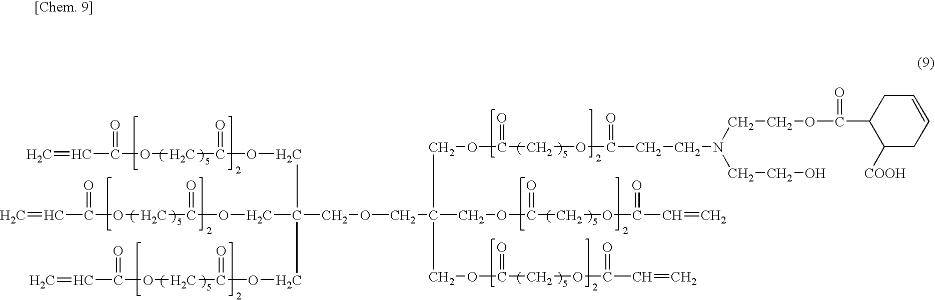

After that, a curable resin composition for a column spacer was prepared in the same manner as Example 14, except that the obtained compound (A) was used in place of the carboxyl-containing caprolactone-modified dipentaerythritol hexaacrylate.

### Example 19

A compound (F) having the structure shown in the following chemical formula (10) was obtained in the same manner as Example 18, except that the addition amount of tetrahydrophthalic anhydride was changed to 2.82 parts by weight (18.6 mmol). The result of NMR measurement of the obtained compound (F) is shown in FIG. 8.

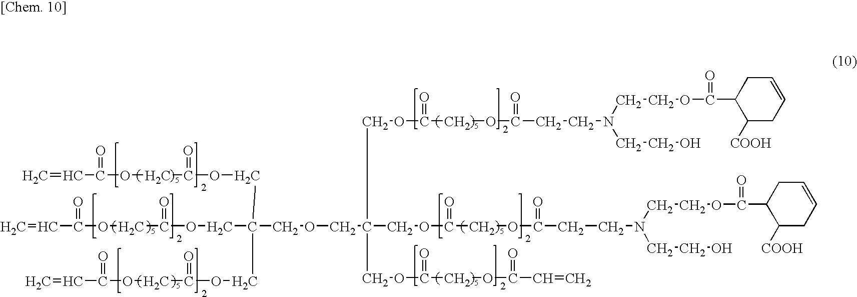

After that, a curable resin composition for a column spacer was prepared in the same manner as Example 14, except that the obtained compound (F) was used in place of the carboxyl-containing caprolactone-modified dipentaerythritol hexaacrylate.

### Example 20

A compound (G) having the structure shown in the following chemical formula (11) was obtained in the same manner as Example 18, except that the addition amount of tetrahydrophthalic anhydride was changed to 4.03 parts by weight (26.5 mmol). The result of NMR measurement of the obtained compound (G) is shown in FIG. 9.

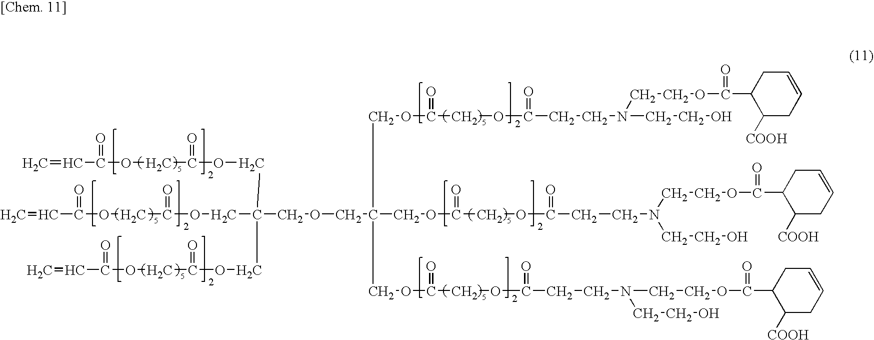

After that, a curable resin composition for a column spacer was prepared in the same manner as Example 14, except that the obtained compound (G) was used in place of the carboxyl-containing caprolactone-modified dipentaerythritol hexaacrylate.

### Example 21

A flask was loaded with 20 parts by weight (15.2 mmol) of a compound obtained by adding diethanolamine to caprolactone-modified pentaerythritol triacrylate shown in the following chemical formula (12) as a raw material monomer, 2.31 parts by weight (15.2 mmol) of tetrahydrophthalic anhydride as an acid anhydride, 0.01 parts by weight of hydroquinone as a polymerization inhibitor, and 20 parts by weight of propylene glycol methyl ether acetate (PGMEA) as a solvent and the mixture was heated under nitrogen flow.

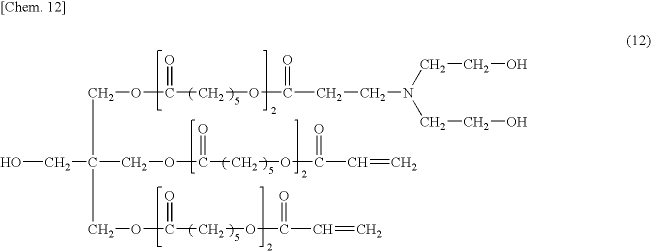

Next, when tetrahydrophthalic anhydride was completely dissolved, 0.02 parts by weight of triethylamine as a catalyst was added and after reaction was carried out in an oil bath at 120° C. for 6 hours under nitrogen atmosphere, the reaction product was cooled to a room temperature to obtain a compound (H) having a structure shown in the following chemical formula (13). The result of NMR measurement of the obtained compound (H) is shown in FIG. 10.

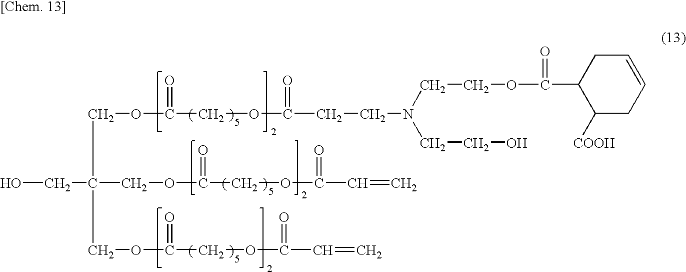

After that, a curable resin composition for a column spacer was prepared in the same manner as Example 14, except that the obtained compound (H) was used in place of the carboxyl-containing caprolactone-modified dipentaerythritol hexaacrylate.

### Example 22

A flask was loaded with 20 parts by weight (12.0 mmol) of the caprolactone-modified dipentaerythritol pentaacrylate having a structure shown in the following chemical formula (14) (a compound obtained by esterification of 1 mole of dipentaerythritol and 5 mole of a compound obtained by reaction of 1 mole of acrylic acid and 2 mole of caprolactone) as a raw material monomer, 1.20 parts by weight (12.0 mmol) of succinic anhydride as an acid anhydride, 0.01 parts by weight of hydroquinone as a polymerization inhibitor, and 20 parts by weight of propylene glycol methyl ether acetate (PGMEA) as a solvent and the mixture was heated under nitrogen flow.

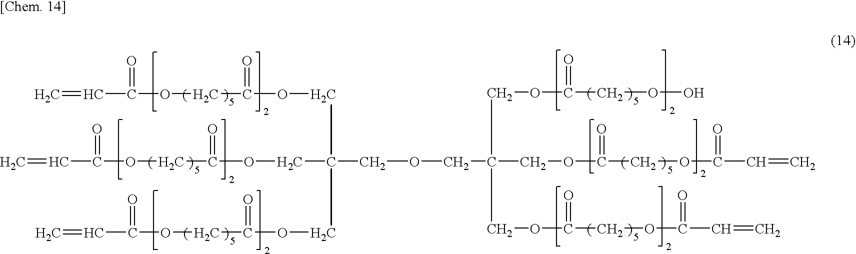

Next, when succinic anhydride was completely dissolved, 0.02 parts by weight of triethylamine as a catalyst was added and after reaction was carried out in an oil bath at 120° C. for 6 hours under nitrogen atmosphere, the reaction product was cooled to a room temperature to obtain a compound (I) having a structure shown in the following chemical formula (15).

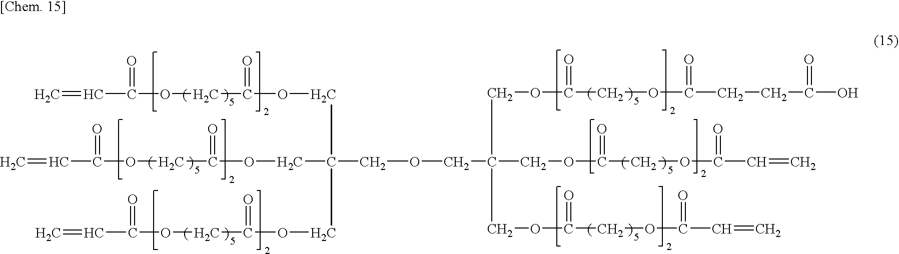

After that, a curable resin composition for a column spacer was prepared in the same manner as Example 14, except that the obtained compound (I) was used in place of the carboxyl-containing caprolactone-modified dipentaerythritol hexaacrylate.

### Example 23

A flask was loaded with 20 parts by weight (16.5 mmol) of the caprolactone-modified pentaerythritol triacrylate having a structure shown in the following chemical formula (16) (a compound obtained by esterification of 1 mole of pentaerythritol and 3 mole of a compound obtained by reaction of 1 mole of acrylic acid and 2 mole of caprolactone) as a raw material monomer, 16.5 parts by weight (16.5 mmol) of succinic anhydride as an acid anhydride, 0.01 parts by weight of hydroquinone as a polymerization inhibitor, and 20 parts by weight of propylene glycol methyl ether acetate (PGMEA) as a solvent and the mixture was heated under nitrogen flow.

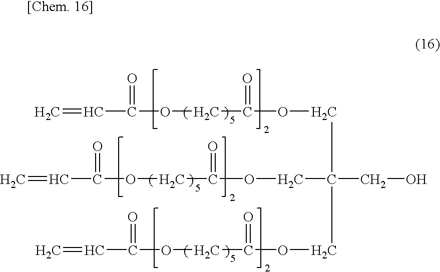

Next, when succinic anhydride was completely dissolved, 0.02 parts by weight of triethylamine as a catalyst was added and after reaction was carried out in an oil bath at 120° C. for 6 hours under nitrogen atmosphere, the reaction product was cooled to a room temperature to obtain a compound (J) having a structure shown in the following chemical formula (17).

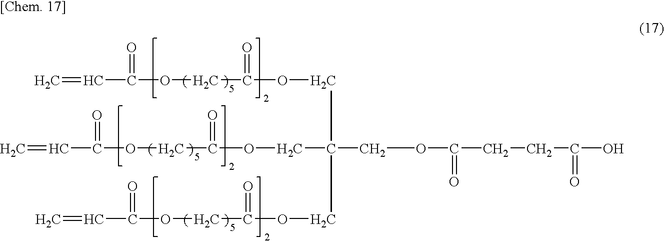

After that, a curable resin composition for a column spacer was prepared in the same manner as Example 14, except that the obtained compound (J) was used in place of the carboxyl-containing caprolactone-modified dipentaerythritol hexaacrylate.

### Comparative Example 4

### Preparation of a Curable Resin Composition

A curable resin composition was prepared by mixing 100 parts by weight of the alkali-soluble polymer compound solution obtained in Example 14, 60 parts by weight of caprolactone-modified dipentaerythritol hexaacrylate (DPCA-120, manufactured by Nippon Kayaku Co., Ltd.) as a polymerizable unsaturated bond-containing compound, 10 parts by weight of Irgacure 907 (manufactured by Ciba Specialty Chemicals Inc.) and 10 parts by weight of DETX-S (manufactured by Nippon Kayaku Co., Ltd.) as a photo-reaction initiator, and 70 parts by weight of diethylene glycol dimethyl ether as a solvent.

### (Evaluation)

The curable resin compositions obtained in Examples 14 to 23 and Comparative Example 4 were evaluated by the same methods as Example 1. The respective results are shown in Table 1.

### Example 24

A flask was loaded with 20 parts by weight (10.8 mmol) of caprolactone-modified dipentaerythritol pentaacrylate having a structure shown in the following chemical formula (18) (a compound obtained by reaction of 1 mole of dipentaerythritol and 12 mole of ε-caprolactone and successive esterification the reaction product and 5 mole of acrylic acid) as a raw material monomer, 1.28 parts by weight (10.8 mmol) of succinic anhydride as an acid anhydride, 0.01 parts by weight of hydroquinone as a polymerization inhibitor, and 20 parts, by weight of propylene glycol methyl ether acetate (PGMEA) as a solvent and the mixture was heated under nitrogen flow. Result of NMR measurement of the caprolactone-modified dipentaerythritol pentaacrylate having a structure shown in the chemical formula (18) and used as the raw material monomer is shown in FIG. 12.

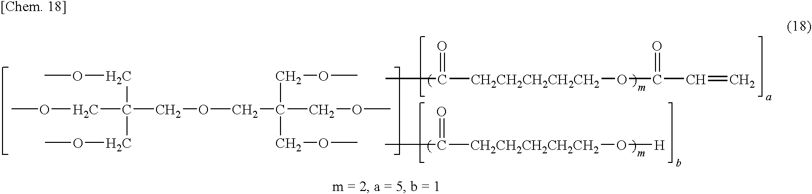

Next, when succinic anhydride was completely dissolved, 0.02 parts by weight of triethylamine as a catalyst was added and after reaction was carried out in an oil bath at 120° C. for 6 hours under nitrogen atmosphere, the reaction product was cooled to a room temperature to obtain a compound (K) having a structure shown in the following chemical formula (19). The result of NMR measurement of the obtained compound (K) is shown in FIG. 13.

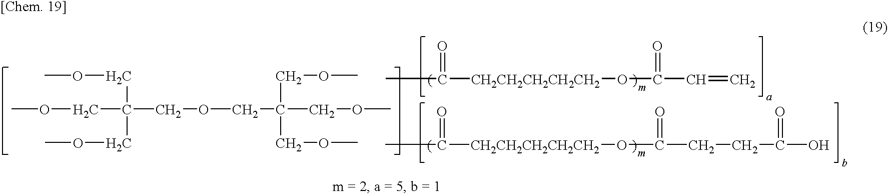

After that, a curable resin composition for a column spacer was prepared in the same manner as Example 14, except that the obtained compound (K) was used in place of the carboxyl-containing caprolactone-modified dipentaerythritol hexaacrylate.

### Example 25

A flask was loaded with 20 parts by weight (16.8 mmol) of caprolactone-modified pentaerythritol triacrylate having a structure shown in the following chemical formula (20) (a compound obtained by reaction of 1 mole of pentaerythritol and 8 mole of ε-caprolactone and successive esterification the reaction product and 3 mole of acrylic acid) as a raw material monomer, 1.98 parts by weight (16.8 mmol) of succinic anhydride as an acid anhydride, 0.01 parts by weight of hydroquinone as a polymerization inhibitor, and 20 parts by weight of propylene glycol methyl ether acetate (PGMEA) as a solvent and the mixture was heated under nitrogen flow. Result of NMR measurement of the caprolactone-modified pentaerythritol triacrylate having a structure shown in the chemical formula (20) and used as the raw material monomer is shown in FIG. 14.

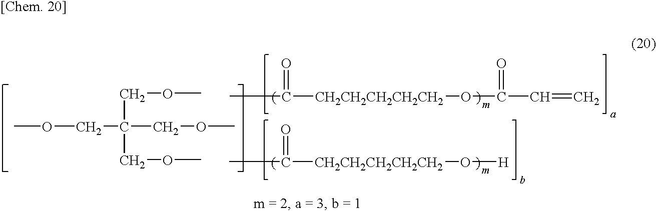

Next, when succinic anhydride was completely dissolved, 0.02 parts by weight of triethylamine as a catalyst was added and after reaction was carried out in an oil bath at 120° C. for 6 hours under nitrogen atmosphere, the reaction product was cooled to a room temperature to obtain a compound (L) having a structure shown in the following chemical formula (21). The result of NMR measurement of the obtained compound (L) is shown in FIG. 15.

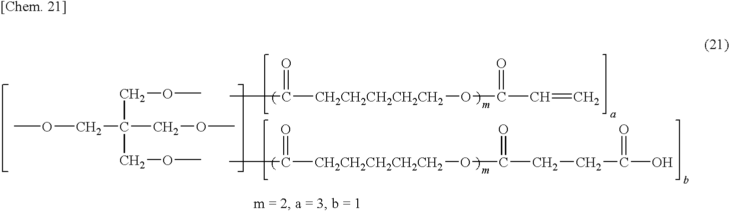

After that, a curable resin composition for a column spacer was prepared in the same manner as Example 14, except that the obtained compound (L) was used in place of the carboxyl-containing caprolactone-modified dipentaerythritol hexaacrylate.

### Example 26

A flask was loaded with 20 parts by weight (18.3 mmol) of ethylene oxide-modified pentaerythritol triacrylate having a structure shown in the following chemical formula (22) (a compound obtained by reaction of 1 mole of pentaerythritol and 20 mole of ethylene oxide and successive esterification the reaction product and 3 mole of acrylic acid) as a raw material monomer, 2.16 parts by weight (18.3 mmol) of succinic anhydride as an acid anhydride, 0.01 parts by weight of hydroquinone as a polymerization inhibitor, and 20 parts by weight of propylene glycol methyl ether acetate (PGMEA) as a solvent and the mixture was heated under nitrogen flow.

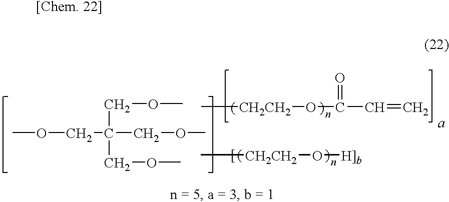

Next, when succinic anhydride was completely dissolved, 0.02 parts by weight of triethylamine as a catalyst was added and after reaction was carried out in an oil bath at 120° C. for 6 hours under nitrogen atmosphere, the reaction product was cooled to a room temperature to obtain a compound (M) having a structure shown in the following chemical formula (23).

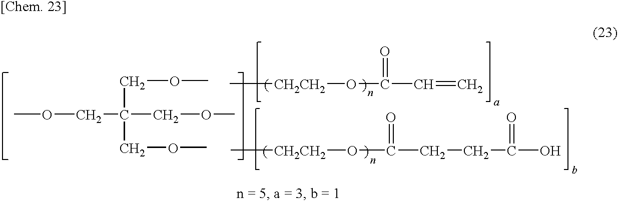

After that, a curable resin composition for a column spacer was prepared in the same manner as Example 14, except that the obtained compound (M) was used in place of the carboxyl-containing caprolactone-modified dipentaerythritol hexaacrylate.

### Example 27

A curable resin composition for a column spacer was prepared in the same manner as Example 14, except that the caprolactone-modified dipentaerythritol pentaacrylate having a structure shown in the chemical formula (18) and obtained in Example 24 was used in place of the carboxyl-containing caprolactone-modified dipentaerythritol hexaacrylate.

### Example 28

A curable resin composition for a column spacer was prepared in the same manner as Example 14, except that the caprolactone-modified dipentaerythritol pentaacrylate having a structure shown in the formula (20) and obtained in Example 25 was used in place of the carboxyl-containing caprolactone-modified dipentaerythritol hexaacrylate.

### (Evaluation)

The curable resin compositions obtained in Examples 24 to 28 were evaluated by the same methods as Example 1. The respective results are shown in Table 1.

TABLE 1

| | column spacer evaluation | | | | | | | |
|---|---|---|---|---|---|---|---|---|
| | | | | compressive | | liquid crystal display panel evaluation | | |
| | pattern | | alkali- | elastic | recovery | cell | gravity | cold |
| | formation | resolution | solubility | coefficient | ratio | gap | defect | bubble |
| Example 1 | ◯ | ◯ | Δ | 0.53 GPa | 70.5% | ◯ | ◯ | ◯ |
| Example 2 | ◯ | ◯ | Δ | 0.57 GPa | 67.8% | ◯ | ◯ | ◯ |
| Example 3 | ◯ | ◯ | ◯ | 0.60 GPa | 66.7% | ◯ | ◯ | ◯ |
| Example 4 | ◯ | ◯ | Δ | 0.60 GPa | 63.4% | ◯ | ◯ | ◯ |
| Example 5 | ◯ | ◯ | Δ | 0.51 GPa | 65.2% | ◯ | ◯ | ◯ |
| Example 6 | ◯ | ◯ | Δ | 0.63 GPa | 64.1% | ◯ | ◯ | ◯ |
| Example 7 | ◯ | ◯ | ◯ | 0.62 GPa | 67.5% | ◯ | ◯ | ◯ |
| Example 8 | ◯ | ◯ | ◯ | 0.66 GPa | 68.5% | ◯ | ◯ | ◯ |
| Example 9 | ◯ | ◯ | ◯ | 0.61 GPa | 66.5% | ◯ | ◯ | ◯ |
| Example 10 | ◯ | ◯ | ◯ | 0.65 GPa | 70.5% | ◯ | ◯ | ◯ |
| Example 11 | ◯ | ◯ | ◯ | 0.63 GPa | 68.3% | ◯ | ◯ | ◯ |
| Example 12 | ◯ | ◯ | ◯ | 0.61 GPa | 67.8% | ◯ | ◯ | ◯ |
| Example 13 | ◯ | ◯ | ◯ | 0.55 GPa | 73.5% | ◯ | ◯ | ◯ |
| Comparative | ◯ | ◯ | X | 0.60 GPa | 66.7% | ◯ | ◯ | ◯ |
| Example 1 | | | | | | | | |
| Comparative | ◯ | ◯ | Δ | 1.24 GPa | 86.2% | ◯ | X | X |
| Example 2 | | | | | | | | |
| Comparative | ◯ | ◯ | ⊚ | 1.10 GPa | 83.7% | ◯ | X | X |
| Example 3 | | | | | | | | |
| Example 14 | ◯ | ◯ | ⊚ | 0.57 GPa | 70.5% | ◯ | ◯ | ◯ |
| Example 15 | ◯ | ◯ | ⊚ | 0.60 GPa | 72.3% | ◯ | ◯ | ◯ |
| Example 16 | ◯ | ◯ | ⊚ | 0.55 GPa | 69.7% | ◯ | ◯ | ◯ |
| Example 17 | ◯ | ◯ | ⊚ | 0.50 GPa | 69.4% | ◯ | ◯ | ◯ |
| Example 18 | ◯ | ◯ | ⊚ | 0.62 GPa | 65.7% | ◯ | ◯ | ◯ |
| Example 19 | ◯ | ◯ | ⊚ | 0.65 GPa | 68.0% | ◯ | ◯ | ◯ |
| Example 20 | ◯ | ◯ | ⊚ | 0.59 GPa | 66.3% | ◯ | ◯ | ◯ |
| Example 21 | ◯ | ◯ | ⊚ | 0.68 GPa | 64.7% | ◯ | ◯ | ◯ |
| Example 22 | ◯ | ◯ | ⊚ | 0.58 GPa | 71.0% | ◯ | ◯ | ◯ |
| Example 23 | ◯ | ◯ | ⊚ | 0.52 GPa | 72.0% | ◯ | ◯ | ◯ |
| Comparative | ◯ | ◯ | X | 0.58 GPa | 71.7% | ◯ | ◯ | ◯ |
| Example 4 | | | | | | | | |
| Example 24 | ◯ | ◯ | ⊚ | 0.62 GPa | 66.8% | ◯ | ◯ | ◯ |
| Example 25 | ◯ | ◯ | ⊚ | 0.65 GPa | 67.1% | ◯ | ◯ | ◯ |
| Example 26 | ◯ | ◯ | ⊚ | 0.58 GPa | 65.2% | ◯ | ◯ | ◯ |
| Example 27 | ◯ | ◯ | ◯ | 0.59 GPa | 65.3% | ◯ | ◯ | ◯ |
| Example 28 | ◯ | ◯ | ◯ | 0.63 GPa | 65.5% | ◯ | ◯ | ◯ |

### INDUSTRIAL APPLICABILITY

According to the invention, it is made possible to provide a curable resin composition for a column spacer which has excellent developability and solubility and is capable of forming a clearly patterned column spacer without leaving a development residue at the time of pattern formation of the column spacer to be used in producing a liquid crystal display panel; a curable resin composition for a column spacer which is capable of forming a clearly patterned column spacer without leaving a development residue at the time of pattern formation of the column spacer to be used in producing a liquid crystal display panel and capable of obtaining a liquid crystal display panel capable of effectively suppressing occurrence of color irregularity due to gravity defect without generating cold bubble; a column spacer obtained by using the curable resin composition for a column spacer; and a liquid crystal display panel.

### BRIEF DESCRIPTION OF THE DRAWINGS

FIG. 1: A graph showing the NMR measurement result of compound (A) obtained in Example 10;

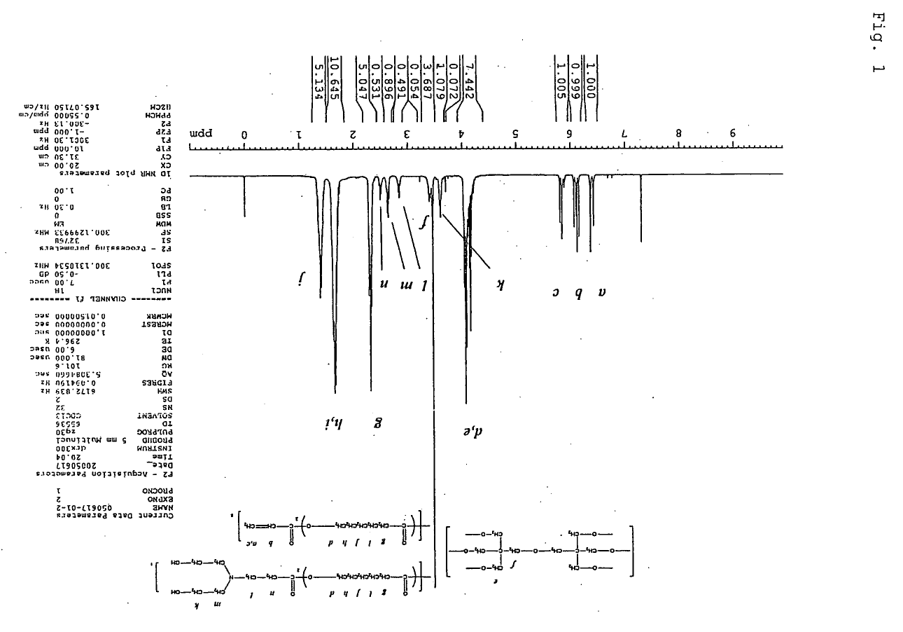

FIG. 2: A graph showing the NMR measurement result of compound (B) obtained in Example 11;

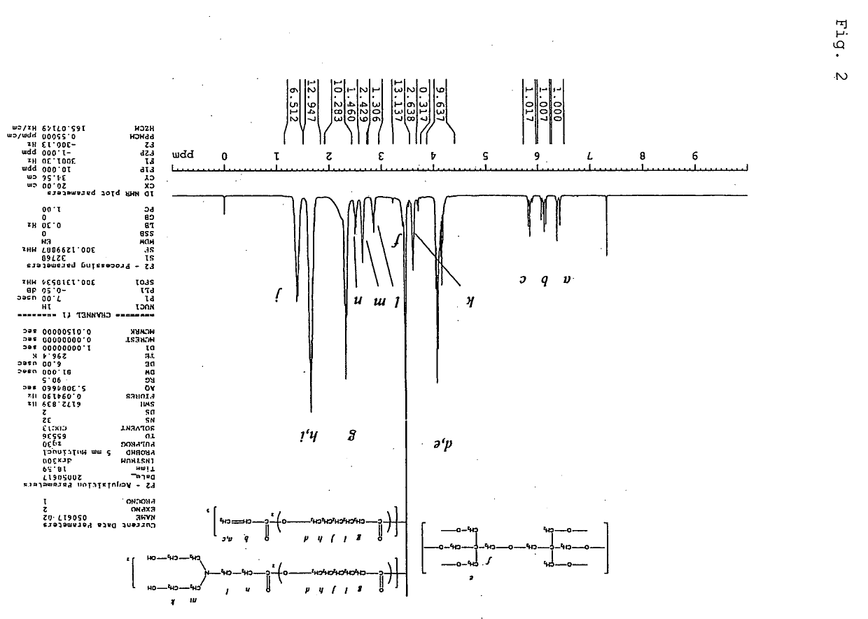

FIG. 3: A graph showing the NMR measurement result of compound (C) obtained in Example 12;

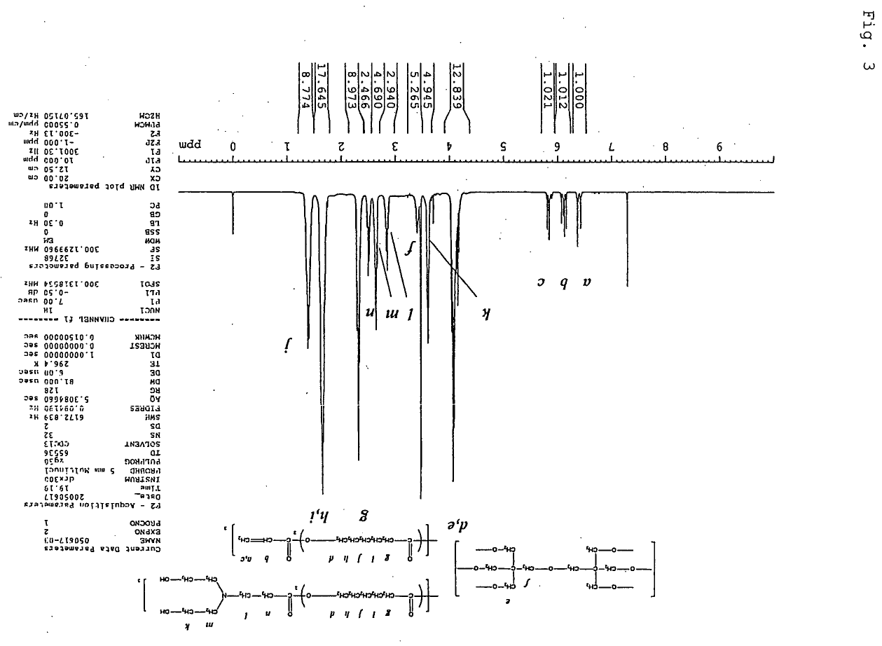

FIG. 4: A graph showing the NMR measurement result of compound (D) obtained in Example 13;

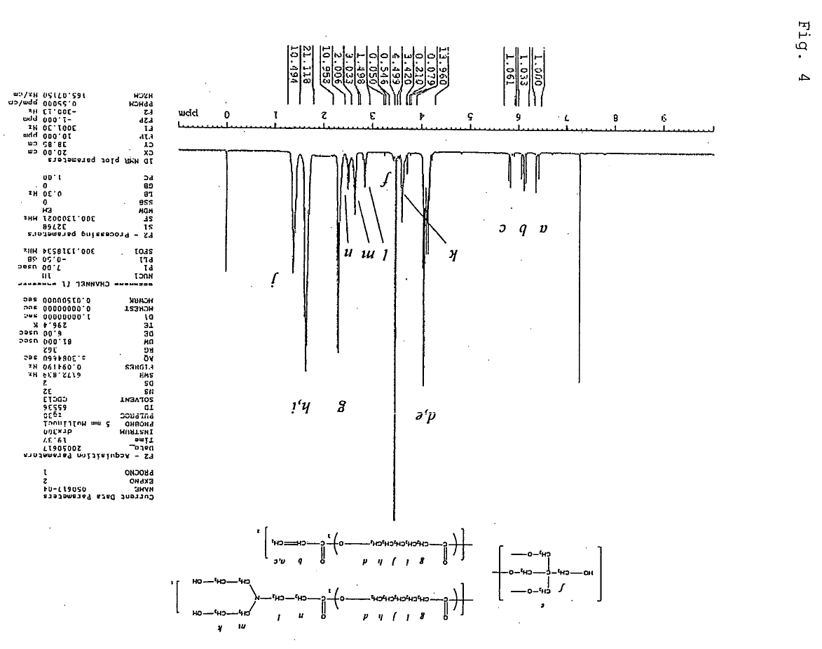

FIG. 5: A graph showing the NMR measurement result of the raw material monomer used in Example 10;

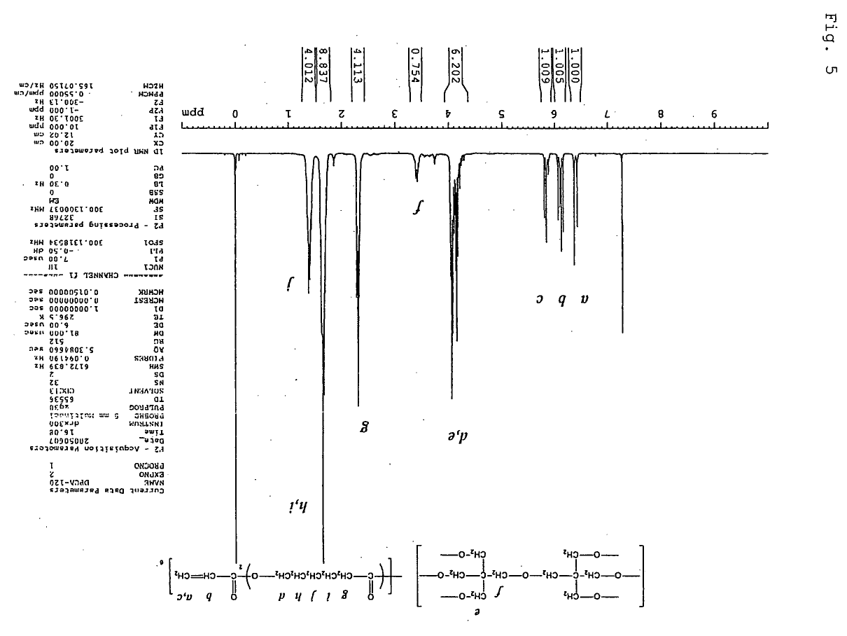

FIG. 6: A graph showing the NMR measurement result of the raw material monomer used in Example 13;

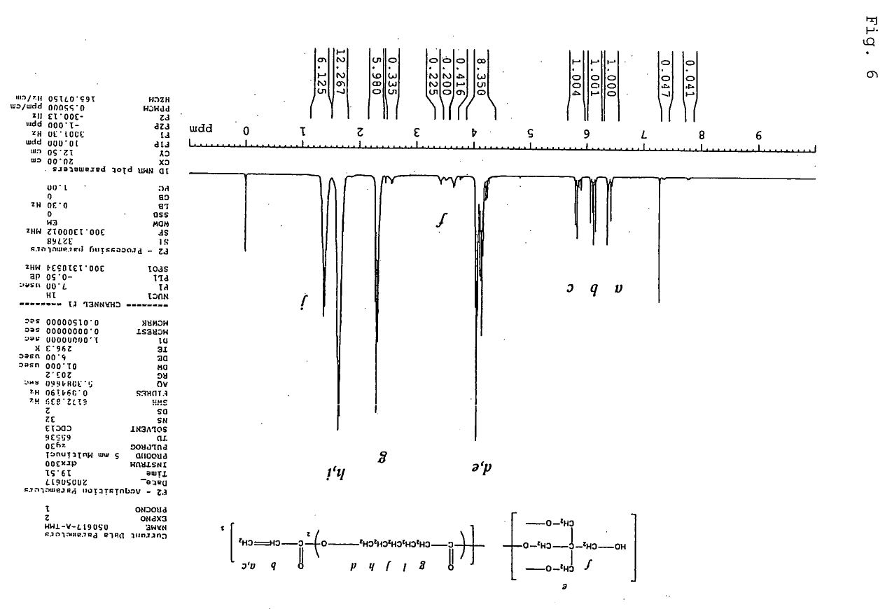

FIG. 7: A graph showing the NMR measurement result of compound (E) obtained in Example 18;

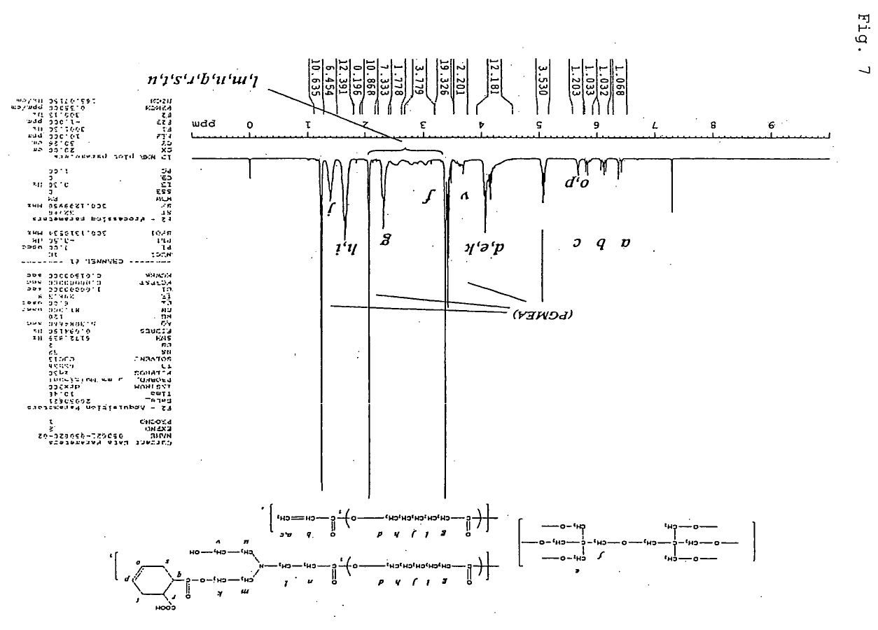

FIG. 8: A graph showing the NMR measurement result of compound (F) obtained in Example 19;

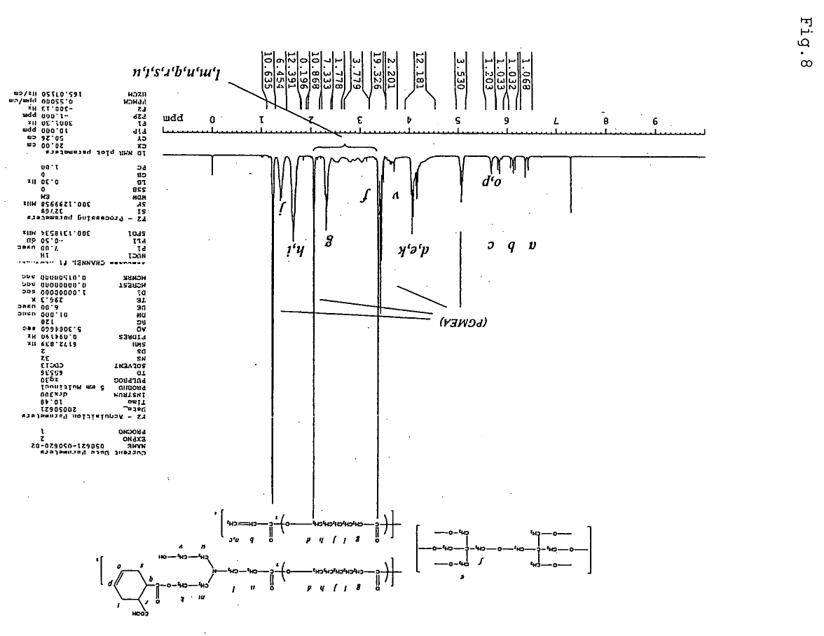

FIG. 9: A graph showing the NMR measurement result of compound (G) obtained in Example 20;

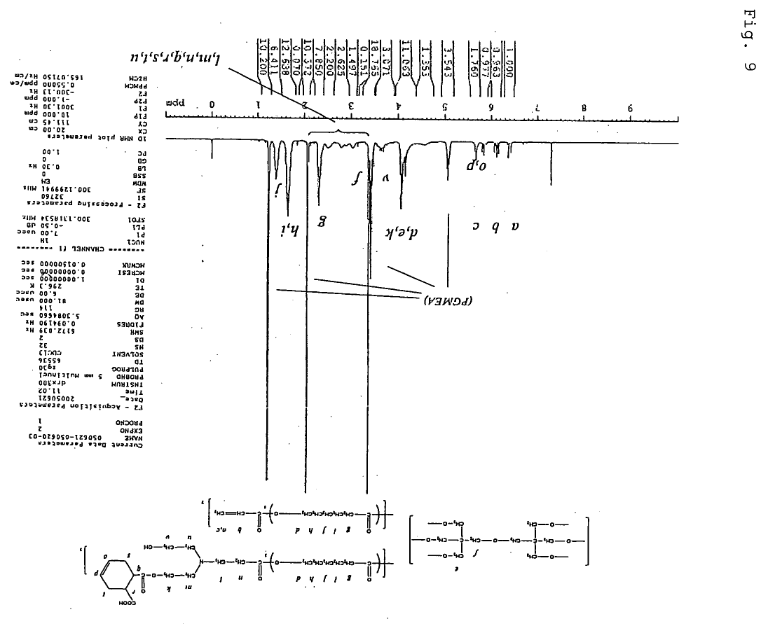

FIG. 10: A graph showing the NMR measurement result of compound (H) obtained in Example 21;

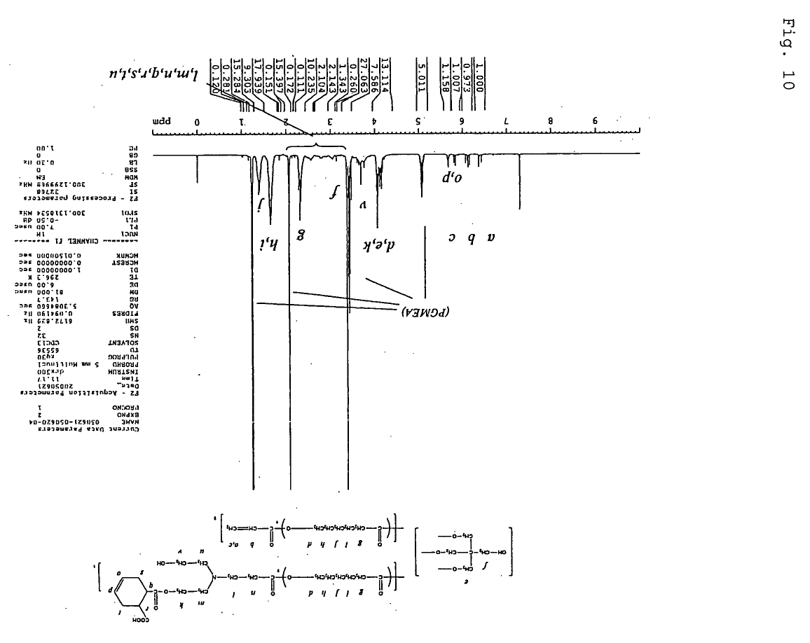

FIG. 11: A graph showing the NMR measurement result of tetrahydrophthalic anhydride used in Example 18;

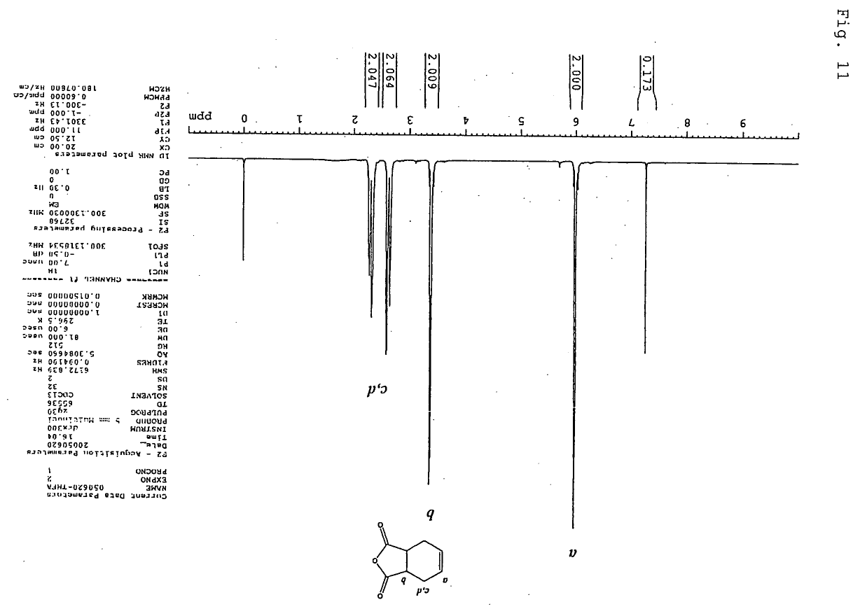

FIG. 12: A graph showing the NMR measurement result of the raw material monomer used in Example 24;

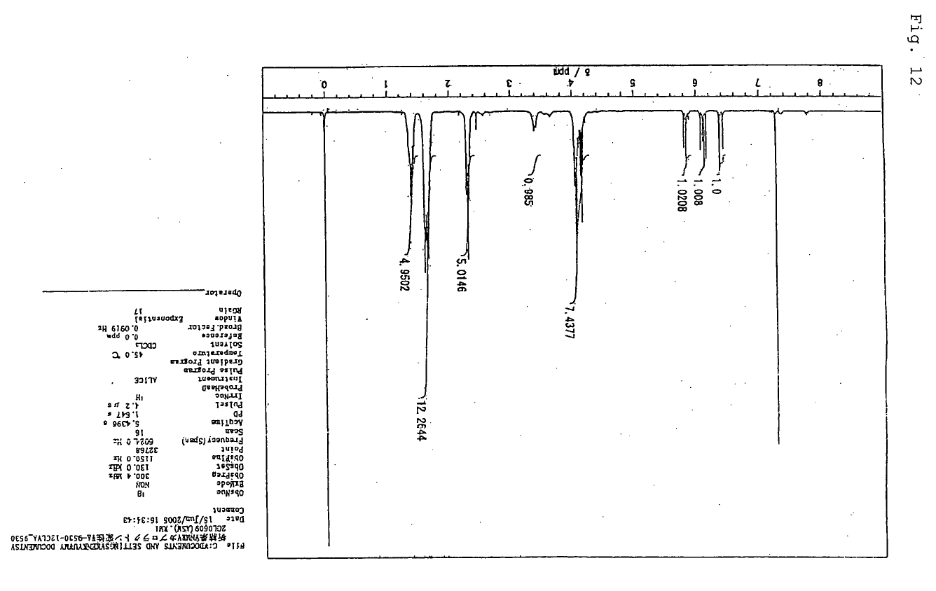

FIG. 13: A graph showing the NMR measurement result of compound (K) obtained in Example 24;

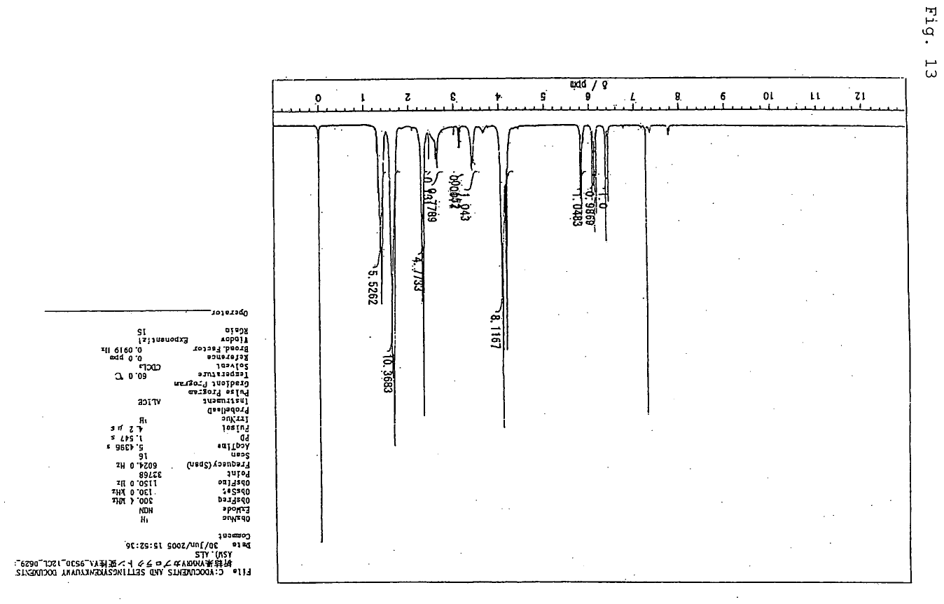

FIG. 14: A graph showing the NMR measurement result of the raw material monomer used in Example 25; and

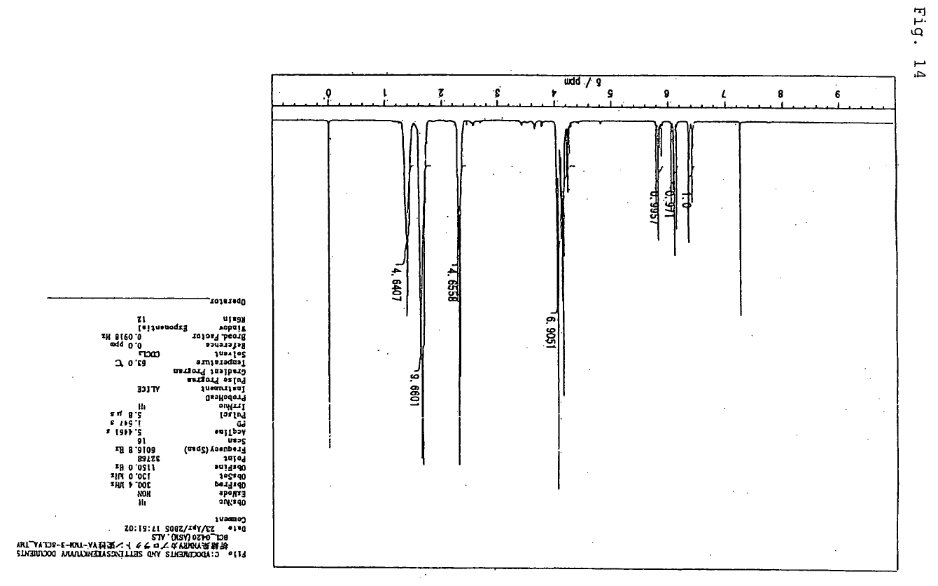

FIG. 15: A graph showing the NMR measurement result of compound (L) obtained in Example 25;

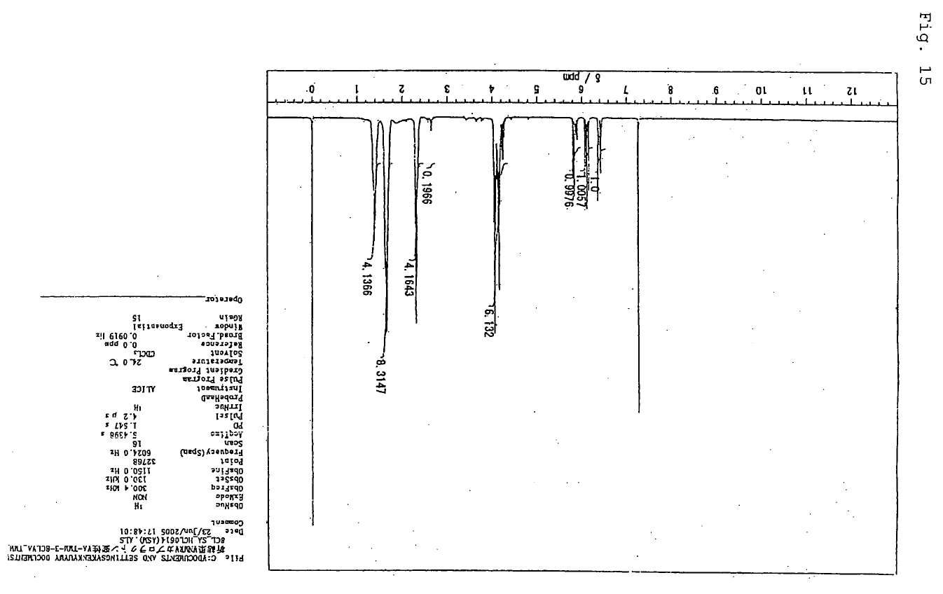

1-24. (canceled)

25. A curable resin composition for a column spacer,
which contains a compound having two or more polymerizable unsaturated bonds in a molecule, an alkali-soluble polymer compound, and a photo-reaction initiator,
the compound having two or more polymerizable unsaturated bonds in a molecule being an oxide-modified compound having two or more polymerizable unsaturated bonds in a molecule,
a cured product obtained by curing by light radiation and/or heating having an elastic coefficient of 0.2 to 1.0 GPa at 25° C. and 15% compression.

26. The curable resin composition for a column spacer of claim 25,
wherein the oxide-modified compound having two or more polymerizable unsaturated bonds in a molecule is an oxide-modified polyfunctional (meth)acrylate compound.

27. A curable resin composition for a column spacer,
which contains a compound having two or more polymerizable unsaturated bonds in a molecule, an alkali-soluble polymer compound, and a photo-reaction initiator,
the compound having two or more polymerizable unsaturated bonds in a molecule being an oxide-modified compound having one or more hydroxyl group and two or more polymerizable unsaturated bonds in a molecule.

28. The curable resin composition for a column spacer of claim 27,
wherein the oxide-modified compound having one or more hydroxyl group and two or more polymerizable unsaturated bonds in a molecule is an oxide-modified polyfunctional (meth)acrylate compound having one or more hydroxyl group in a molecule.

29. The curable resin composition for a column spacer of claim 28,
wherein the oxide-modified polyfunctional (meth)acrylate compound having one or more hydroxyl group in a molecule is a compound obtained by oxide-modifying pentaerythritol tri(meth)acrylate, ditrimethylolpropane tri(meth)acrylate, dipentaerythritol tri(meth)acrylate, dipentaerythritol tetra(meth)acrylate, or dipentaerythritol penta(meth)acrylate.

30. A curable resin composition for a column spacer,
which contains a compound having two or more polymerizable unsaturated bonds in a molecule, an alkali-soluble polymer compound, and a photo-reaction initiator,
the compound having two or more polymerizable unsaturated bonds in a molecule being a lactone-modified and oxide-modified compound having two or more polymerizable unsaturated bonds in a molecule.

31. The curable resin composition for a column spacer of claim 30,
wherein the lactone-modified and oxide-modified compound having two or more polymerizable unsaturated bonds in a molecule is a lactone-modified and oxide-modified polyfunctional (meth)acrylate compound.

32. A curable resin composition for a column spacer,
which contains a compound having two or more polymerizable unsaturated bonds in a molecule, an alkali-soluble polymer compound, and a photo-reaction initiator,
the compound having two or more polymerizable unsaturated bonds in a molecule being a lactone-modified compound having one or more hydroxyl group and two or more polymerizable unsaturated bonds in a molecule.

33. The curable resin composition for a column spacer of claim 32,
wherein the lactone-modified compound having one or more hydroxyl group and two or more polymerizable unsaturated bonds in a molecule is a lactone-modified polyfunctional (meth)acrylate compound having one or more hydroxyl group in a molecule.

34. The curable resin composition for a column spacer of claim 33,
wherein the lactone-modified polyfunctional (meth)acrylate compound having one or more hydroxyl group in a molecule is a compound obtained by lactone-modifying pentaerythritol tri(meth)acrylate, ditrimethylolpropane tri(meth)acrylate, dipentaerythritol tri(meth)acrylate, dipentaerythritol tetra(meth)acrylate, or dipentaerythritol penta(meth)acrylate.

35. A curable resin composition for a column spacer,
which contains a compound having two or more polymerizable unsaturated bonds in a molecule, an alkali-soluble polymer compound, and a photo-reaction initiator,
the compound having two or more polymerizable unsaturated bonds in a molecule being a lactone-modified and oxide-modified compound having one or more hydroxyl group and two or more polymerizable unsaturated bonds in a molecule.

36. The curable resin composition for a column spacer of claim 35,
wherein the lactone-modified and oxide-modified compound having one or more hydroxyl group and two or more polymerizable unsaturated bonds in a molecule is a lactone-modified and oxide-modified polyfunctional (meth)acrylate compound having one or more hydroxyl group in a molecule.

37. The curable resin composition for a column spacer of claim 36,
wherein the lactone-modified and oxide-modified polyfunctional (meth)acrylate compound having one or more hydroxyl group in a molecule is a compound obtained by lactone-modifying and oxide-modifying pentaerythritol tri(meth)acrylate, ditrimethylolpropane tri(meth)acrylate, dipentaerythritol tri(meth)acrylate, dipentaerythritol tetra(meth)acrylate, or dipentaerythritol penta(meth)acrylate.

38. A curable resin composition for a column spacer,
which contains a compound having two or more polymerizable unsaturated bonds in a molecule, an alkali-soluble polymer compound, and a photo-reaction initiator,
the compound having two or more polymerizable unsaturated bonds in a molecule being a compound having one or more carboxyl group and two or more polymerizable unsaturated bonds in a molecule,
a cured product obtained by curing the curable resin composition for a column spacer by light radiation and/or heating having an elastic coefficient of 0.2 to 1.0 GPa at 25° C. and 15% compression.

39. A curable resin composition for a column spacer,
which contains a compound having two or more polymerizable unsaturated bonds in a molecule, an alkali-soluble polymer compound, and a photo-reaction initiator,
the compound having two or more polymerizable unsaturated bonds in a molecule being a lactone-modified and/or oxide-modified compound having one or more carboxyl group and two or more polymerizable unsaturated bonds in a molecule.

40. The curable resin composition for a column spacer of claim 39,
wherein the lactone-modified and/or oxide-modified compound having two or more polymerizable unsaturated bonds in a molecule is a lactone-modified and/or oxide-modified polyfunctional (meth)acrylate compound having one or more carboxyl group in a molecule.

41. The curable resin composition for a column spacer of claim 39,
wherein the lactone-modified and/or oxide-modified polyfunctional (meth)acrylate compound having one or more carboxyl group in a molecule is a compound obtained by adding a compound having a carboxyl group to lactone-modified and/or oxide-modified trimethylolpropane tri(meth)acrylate, trimethylolethane tri(meth)acrylate, pentaerythritol tri(meth)acrylate, pentaerythritol tetra(meth)acrylate, ditrimethylolpropane tri(meth)acrylate, ditrimethylolpropane tetra(meth)acrylate, dipentaerythritol tri(meth)acrylate, dipentaerythritol tetra(meth)acrylate, dipentaerythritol penta(meth)acrylate, or dipentaerythritol hexa(meth)acrylate.

42. The curable resin composition for a column spacer of claim 38 or 39,
wherein the compound having two or more polymerizable unsaturated bonds in a molecule further has one or more hydroxyl group in a molecule.

43. The curable resin composition for a column spacer of claim 38 or 39,
wherein the compound having two or more polymerizable unsaturated bonds in a molecule is a compound obtained by addition reaction of a compound having a carboxyl group and a thiol group to a compound having three or more polymerizable unsaturated bonds in a molecule.

44. The curable resin composition for a column spacer of claim 38 or 39,
wherein the compound having two or more polymerizable unsaturated bonds in a molecule is a compound obtained by addition reaction of a carboxylic acid compound having two or more carboxyl groups and/or an acid anhydride to a compound having two or more polymerizable unsaturated bonds and a hydroxyl group in a molecule.

45. The curable resin composition for a column spacer of claim 25, 27, 30, 32, 35, 38 or 39,
which further contains a compound having two or more block isocyanate groups.

46. A column spacer,
which comprises the curable resin composition for a column spacer of claim 25, 27, 30, 32, 35, 38 or 39.

47. The column spacer of claim 46,
which has an elastic coefficient of 0.2 to 1.0 GPa at 25° C. and 15% compression.

48. A liquid crystal display panel,
which comprises the curable resin composition for a column spacer of claim 25, 27, 30, 32, 35, 38 or 39.

49. A polymerizable compound,
which has two or more polymerizable unsaturated bonds, one or more carboxyl group; a ring-opened structure or ring-opened polymer structure of a lactone and/or a ring-opened structure or ring-opened polymer structure of an oxide in a molecule.

50. The polymerizable compound according to claim 49,
wherein the ring-opened structure or ring-opened polymer structure of a lactone is a ring-opened structure or ring-opened polymer structure of caprolactone, and the ring-opened structure or ring-opened polymer structure of an oxide is a ring-opened structure or ring-opened polymer structure of ethylene oxide and/or propylene oxide.

51. The polymerizable compound according to claim 49,
wherein the polymerizable unsaturated bond is an unsaturated double bond in a (meth)acrylate group.

52. The polymerizable compound according to claim 49,
which is a compound obtained by addition reaction of a carboxylic acid compound having two or more carboxyl groups and/or an acid anhydride to a compound having two or more polymerizable unsaturated bonds, a hydroxyl group; a ring-opened structure or ring-opened polymer structure of a lactone and/or a ring-opened structure or ring-opened polymer structure of an oxide in a molecule.

53. A method of producing a polymerizable compound,
which comprises a step of synthesizing a lactone-modified and/or oxide-modified polyhydric alcohol compound by reaction of a tri- or higher-polyhydric alcohol compound and a lactone and/or an oxide, and a step of adding (meth)acrylic acid, and a carboxylic acid compound having two or more carboxyl groups and/or a carboxylic acid anhydride to the lactone-modified and/or oxide-modified polyhydric alcohol compound.

54. The method of producing a polymerizable compound according to claim 53,
which comprises a step of synthesizing a
caprolactone-modified polyhydric alcohol compound by reaction of a tri- or higher-polyhydric alcohol compound and ε-caprolactone, and a step of esterifying the caprolactone-modified polyhydric with (meth)acrylic acid as one or more hydroxyl group is left, and further adding a carboxyl group by reaction of a carboxylic acid compound having two or more carboxyl groups and/or a carboxylic acid anhydride to the left hydroxyl group.

55. The method of producing a polymerizable compound according to claim 53,
wherein the tri- or higher-polyhydric alcohol compound is at least one kind of compound selected from the group consisting of pentaerythritol, dipentaerythritol, tripentaerythritol, tetrapentaerythritol, trimethylolethane, ditrimethylolethane, trimethylolpropane, and ditrimethylolpropane.
